(19) United States
(12) Patent Application Publication
(10) Pub. No.: US 2008/0306353 A1
(43) Pub. Date: Dec. 11, 2008

(54) CALCULATION DEVICE FOR METABOLIC CONTROL OF CRITICALLY ILL AND/OR DIABETIC PATIENTS

(76) Inventors: Joel S. Douglas, Groton, CT (US); Christopher Eric Hann, Rangiora (NZ); James Geoffrey Chase, Christchurch (NZ); Geoffrey Mark Shaw, Christchurch (NZ); Thomas Friedhelm Lotz, Christchurch (NZ); Timothy Rhys John Lonergan, Christchurch (NZ); Jessica Lin, Christchurch (NZ); Aaron James Le Compte, Christchurch (NZ)

Correspondence Address: MICHAUD-DUFFY GROUP LLP, 306 INDUSTRIAL PARK ROAD, SUITE 206, MIDDLETOWN, CT, 06457, US

(21) Appl. No.: 11/869,322

(22) Filed: Oct. 9, 2007

Related U.S. Application Data
(60) Provisional application No. 60/856,454, filed on Nov. 3, 2006.
(60) Provisional application No. 60/900,003, filed on Feb. 7, 2007.
(60) Provisional application No. 60/905,360, filed on Mar. 7, 2007.

(51) Int. Cl. A61B 5/145; A61B 5/01; A61B 5/02

(52) U.S. Cl. 600/301

(57) ABSTRACT

A method of providing blood glucose therapy for a critically ill patient includes calculating a baseline nutrition feed requirement based on an algorithm that incorporates at least one of age, gender, and body size of the patient: determining a first blood glucose level; determining a second blood glucose level after a preselected time interval: determining a first body temperature reading: comparing the blood glucose levels: and administering either nutrition or insulin. The amount of nutrition administered to the patient is based on a first change in blood glucose level, the current body temperature reading, and a predetermined feed algorithm based on the second blood glucose level as well as the baseline nutritional feed requirement. The amount of insulin administered is based on a second change in blood glucose level, body temperature, and a predetermined insulin algorithm that incorporates at least one of the patient's body frame size, age, and gender.

### CROSS REFERENCE TO RELATED APPLICATIONS

This application claims the benefit of U.S. Provisional Patent Application No. 60/856,454 filed Nov. 3, 2006, U.S. Provisional Patent Application No. 60/900,003 filed Feb. 7, 2007, and U.S. Provisional Patent Application No. 60/905,360 filed Mar. 7, 2007, the contents of all of the foregoing applications being incorporated herein by reference in their entirety.

### TECHNICAL FIELD

The present invention relates generally to calculation devices and, more particularly, to computational devices that utilize measured physiological parameters that are relevant to human metabolism as inputs for determining insulin dosage and nutrition dosage recommendations for a future period of time to assist clinicians in obtaining and maintaining metabolic homeostasis in critically ill patients.

### BACKGROUND OF THE INVENTION

Many foods are carbohydrates, which are converted to glucose by the digestive process and transported throughout the body via the bloodstream. Cells absorb this glucose in order to properly rebuild older tissue and support life. Insulin, a hormone produced by the pancreas, is a key factor in the absorption of glucose by the cells. Without this hormone being present, many cells will not absorb glucose adequately, thereby impairing cell function.

In a person undergoing intensive care therapy (for example, as a patient in an intensive care unit (ICU) in a hospital), the metabolic condition of the person is often distressed due to hormonal imbalances. In critically ill patients, hyperglycemia and impaired metabolic function is prevalent, even in patients with no prior diabetic condition. When hormones, particularly insulin, are out of balance, any resulting impairment of the cell function may further result in the development of a compromising health condition. In particular, the patient may experience difficulty in utilizing the glucose or insulin, or they may encounter difficulty in producing insulin because of their distressed metabolic condition. In such cases, the healing process is often negatively affected.

Critically ill patients often experience stress-induced hyperglycemia and high levels of insulin resistance, even given no history of diabetes. The need for a convenient and easily applied method of metabolic monitoring in the ICU became evident after the landmark study of Van den Berghe and colleagues published in the Nov. 8, 2001, issue of The New England Journal of Medicine. This paper demonstrated an overall reduction in ICU patient mortality of 34% when blood glucose was kept in the 4.4-6.1 mmol/L range. A virtual flood of articles have since appeared and confirm improved outcomes in the treatment of various critical conditions including infection, stroke, in patients undergoing coronary bypass surgery, and in the treatment of myocardial infarction in both diabetic and non-diabetic patients. One study showed greatly improved outcomes when diabetics were monitored and treated intensively with insulin in the hospital for three days prior to undergoing coronary bypass surgery. Additionally, tight blood glucose control has been associated with reduced requirements for prolonged mechanical ventilation (Hermans, Greet et al., “Impact of Intensive Insulin Therapy on Neuromuscular Complications and Ventilator Dependency in Medical Intensive Care Unit.” American Journal of Respiratory and Critical Care Medicine; Mar. 1, 2007; 175, 5; Health & Medical Complete pg. 480).

U.S. Patent Application No. 20020107178 to Van den Berghe discloses methods used to cure critically ill patients. The methods incorporate the delivery of a pharmaceutically effective amount of a blood glucose regulator to a critically ill patient to control blood glucose levels. The use of blood glucose regulators, such as insulin, to treat critically ill patients has been practiced in intensive care units for decades. However, there exists a need for an easily implemented system that accounts for the complex factors that affect the glucose levels of a patient and can accurately titrate amounts of insulin and other medication known to affect the human metabolism to match the metabolic state and demand of the individual.

The medical community uses a scale of 0-33 millimoles per liter (hereinafter “mmol/L”) to represent the concentration of glucose in the blood. Low blood glucose is considered to be 0-3.9 mmol/L. Normal blood glucose is considered 4-6.9 mmol/L. High blood glucose is in the range 7-11 mmol/L. Very high blood glucose is over 11.1 mmol/L. For a patient in the ICU a glucose level over 11 mmol/L has negative affect on healing and the process of assisted ventilation.

The condition of having very low blood glucose is also known as hypoglycemia. In hypoglycemic patients, brain damage and death can occur if immediate action is not taken to elevate the blood glucose to a normal or at least near-normal level. The condition of having very high blood glucose is known as hyperglycemia. As the blood glucose level approaches 33 mmol/L, brain damage and death can occur if immediate corrective action is not taken. An ICU patient experiencing either hypoglycemia or hyperglycemia can quickly become comatose, which even further negatively affects the healing process. They also experience a decreased response to other therapies such as ventilation and are more likely to experience ventilator-induced pneumonia.

To correct a hypoglycemic condition, glucose is supplied to the blood immediately, by orally ingesting any carbohydrate (including sugar). In severe cases where the patient becomes unconscious, a glucagon injection may be necessary. Once the carbohydrate is ingested or the glucagon is injected, the blood glucose will typically rise within minutes.

As stated above, normal blood glucose levels are between 4-6.9 mmol/L. The range of 6.9-11 mmol/L can be considered a buffer zone, but blood glucose levels between 11 mmol/L and up to and over 28 mmol/L may be particularly harmful. Long-term blood glucose levels in this range can cause blindness, kidney failure, and nerve damage, as well as increasing the risk of heart attacks four-fold. Allowing blood glucose to go over 11 mmol/L is dangerous because excess glucose will be flushed from the blood and out through the kidneys with urine. This level of 11 mmol/L is known in medical terms as the “renal threshold” with the word “renal” meaning “kidney.” This is the point at which the glucose dumps or spills from the blood and is carried through the kidney and into the bladder. For a patient in the ICU, a blood glucose level over 11 mmol/L has negative affect on healing and the process of assisted mechanical ventilation. A patient in the ICU suffering from poor glycemic control has a decreased response to other therapies such as mechanical ventilation. This is extremely problematic for a patient on mechanical ventilation due to their already compromised condition and the issues with mechanical ventilator induced pneumonia. The inability to heal quickly and ward off infection makes mechanical ventilator induced pneumonia a significant concern. By quickly getting a patient under glycemic control, the patient healing and resistance to infection is increased thereby reducing the opportunity for getting secondary infections such as mechanical ventilator-induced pneumonia.

In addition to mechanical ventilation units, multiple devices such fluid pumps connected to feeding or fluid tubes and analyte sensors are employed in critical care medicine to promote healing. In intensive care metabolic management, nutrition delivery devices, insulin delivery devices, and body fluid analyte devices are all used routinely in patient care.

One type of nutrition delivery device is a feeding tube. A feeding tube is a medical device used to provide nutrition to patients who cannot obtain nutrition by swallowing. The state of being fed by a feeding tube is called enteral feeding or tube feeding. Placement may be temporary for the treatment of acute conditions or lifelong in the case of chronic disabilities. Many patients, such as critically ill patients in an intensive care unit, are treated using a feeding tube lack the ability to survive on their own without such technology.

The feeding tube may be a nasogastric feeding tube, a gastric feeding, or a jejunostomy tube. A nasogastric feeding tube, or “NG-tube,” is passed through the nares, down the esophagus and into the stomach. A gastric feeding tube, or “G-tube”, is a tube inserted through a small incision in the abdomen into the stomach and is used for long-term enteral nutrition. Gastrostomy tubes can also be placed in “open” procedures through an incision with direct visualization of the stomach, as well as via laparoscope. Gastric tubes are suitable for long-term use: they last about six months, and can be replaced through an existing passage without an additional endoscopic procedure. The G-tube is useful where there is difficulty with swallowing because of neurologic or anatomic disorders, and to avoid the risk of aspiration pneumonia. A jejunostomy tube is similar to a gastric tube, though generally has a finer bore and smaller diameter, and is surgically inserted into the jejunum rather than the stomach. They are used when the upper gastrointestinal tract must be bypassed completely, and can be used as soon as 12 hours after surgery. This type of tube is usually used for people who are at high risk for aspiration.

The selected feeding tube is used with an infusion pump to infuse nutrients to a patient's circulatory system. Infusion pumps can administer fluids in ways that would be impractically expensive or unreliable if performed manually by nursing staff. For example, they can administer as little as 0.1 mL per hour injections (too small for a drip), injections every minute, injections with repeated boluses requested by the patient, up to maximum number per hour, or fluids whose volumes vary by the time of day. The user interface of pumps usually requests details on the type of infusion from the technician or nurse that sets them up. For example the nurse can program the duration and rate of infusion. In this manner the nurse is in complete control of the amount of nutrition received by the patient. Hence the nursing staff has the ability to completely customize the nutrition regime for each patient and evolve this regime over time to match patient nutritional demands and changing patient condition.

If the patient is having trouble receiving nutrition by the gastrointestinal tract, total parenteral nutrition (TPN), is used and is the practice of feeding a person intravenously, bypassing the usual process of eating and digestion. The person receives nutritional formulas containing salts, glucose, amino acids, lipids and added vitamins.

TPN is normally used following surgery, when feeding by mouth or using the gastrointestinal tract is not possible, when a person's digestive system cannot absorb nutrients due to chronic disease, or, alternatively, if a person's nutrient requirement cannot be met by enteral feeding (tube feeding) and supplementation. It has been used for comatose patients, although enteral feeding is usually preferable, and less prone to complications. Short-term TPN may be used if a person's digestive system has shut down (for instance by peritonitis), and they are at a low enough weight to cause concerns about nutrition during an extended hospital stay. Long-term TPN is occasionally used to treat people suffering the extended consequences of an accident or surgery. Most controversially, TPN has extended the life of a small number of children born with nonexistent or severely birth-deformed gastrointestinal tracts. The oldest were eight years old in 2003.

The preferred method of delivering TPN is with a medical infusion pump. A sterile bag of nutrient solution, between 500 mL and 4 L is provided. The pump infuses a small amount (0.1 to 10 mL/hr) continuously in order to keep the vein open. Feeding schedules vary, but one common regimen ramps up the nutrition over a few hours, levels off the rate for a few hours, and then ramps it down over a few more hours, in order to simulate a normal set of meal times. The technician, nurse, or other caregiver has complete control over the medical infusion pump and can program the infusion pump to deliver a customized nutrition profile. It is common practice for a nurse to routinely change the infusion rate over the patient length of stay.

Unlike many other medicines, insulin cannot be taken orally. Like nearly all other proteins introduced into the gastrointestinal tract, it is reduced to fragments (even single amino acid components), whereupon all ‘insulin activity’ is lost. Insulin is usually taken as subcutaneous injections by single-use syringes with needles, an insulin pump, an infusion pump, or by repeated-use insulin pens with needles.

In a clinical environment, such as the ICU, drug delivery devices are commonplace and are used to give bolus or continuous infusions of multiple drugs such as insulin. In critical care medicine, insulin is predominately delivered to patient via an infusion pump connected to an intravenous line. Currently, the insulin infusion rate is set by the technician, nurse, or other caregiver on the infusion pump such that this person has absolute control over the amount of insulin received by the patient. Incorporating a step where nursing staff must confirm the insulin dosage is a safety feature. If insulin delivery was completely closed-loop, a malfunction resulting in over delivery of insulin could result in severe hypoglycaemia, brain damage, and even death. From a safety perspective, it is essential the technician, nurse, or other caregiver must confirm each insulin dosage amount before delivery to the patient.

A glucose meter (or glucose sensor) is a medical device for determining the approximate concentration of glucose in the blood. Multiple technologies including simple test strips, blood glucose meters, continuous blood glucose monitors are currently used in different patient groups. In intensive care units blood glucose meters and continuous blood glucose monitors are predominately employed.

A blood glucose meter is an electronic device for measuring the blood glucose level. A relatively small drop of blood is placed on a disposable test strip which interfaces with a digital meter. Within several seconds, the level of blood glucose will be shown on the digital display. A continuous blood glucose monitor determines blood glucose levels on a continuous basis (every few minutes). A typical system consists of a disposable glucose sensor placed just under the skin, which is worn for a few days until replacement, a link from the sensor to a non-implanted transmitter which communicates to a radio receiver, and an electronic receiver worn like a pager (or insulin pump) that displays blood glucose levels on a practically continuous manner, as well as monitors rising and falling trends in glycemic excursions.

Continuous blood glucose monitors measure the glucose level of interstitial fluid. Some new technologies to monitor blood glucose levels will not require access to blood to read the glucose level. Non-invasive technologies include near IR detection, ultrasound and dielectric spectroscopy, all of which may be used in the future to monitor the blood glucose levels of patients in intensive care units.

Additional sensors may also be used in intensive care units to test for alternative body fluid analytes. Alternative analyte measurements provide information indicative of the current patient chemistry and evolving patient condition. Additional body fluid analytes include but are not limited to albumin, ALKP, ALT, Ammonia, Amylase serum, Amylase urine, AST, bilirubin, BNP, CA125, calcium serum, calcium urine, carbon dioxide, carboxyhemoglobin, CEA, chloride, creatinine, DHEA sulfate, estradiol, ferritin, folate, FSH, GGT, HDL cholesterol, hematocrit, hemoglobin, homocysteine, lactate, lactic acid, lead, lipase, lut hormone, magnesium serum, methemoglobin, microalbumin, myoglobin, oxyhemoglobin, osmolality, pCO₂, pH, phosphorus serum, potassium serum, prolactin, RBC folate, testosterone, transferrin, troponin I, urea nitrogen, and uric acid.

Currently these three sets of devices, (nutrition delivery, insulin delivery, and body fluid analyte sensors) are operated independently by intensive care nursing staff. For example the nurse must manually perform the glucose test and then manually dial in the nutrition and insulin rates on the respective delivery pumps.

Maintaining a blood glucose concentration in the 4-6 mmol/L range provides a cushion to protect the patient from a dangerous low blood glucose condition. As the blood glucose concentration increases and surpasses the high end of this range, the patient gets sleepy and lethargic but otherwise feels quite normal. Therefore, while a reasonable goal is to keep blood glucose levels in the 4-6 mmol/L range, a range of 4-7.75 mmol/L is acceptable.

Intensive care patients experience states in which their blood glucose levels are compromised due to the stresses placed on their bodies and metabolic systems. A patient in the ICU suffering from poor glycemic control has the negative effect of retarded recovery, increased organ damage, and decreased response to other therapies such as controlled mechanical ventilation. For a person suffering from poor glycemic control, there are four major targets that can be attributed directly to excess blood glucose. The first of these targets is the heart and the arteries. As excess glucose accumulates in the bloodstream, it causes the walls of the arteries to harden (a condition called atherosclerosis). This hardening will eventually contribute to clogging of arteries, leading to an increased chance of heart attack. Persons with uncontrolled or poorly controlled blood glucose levels have a four times greater risk of heart attack than persons with normal blood glucose levels. The risk of stroke is also significantly increased.

The second of the above-described targets is the kidneys. When blood glucose concentration reaches 11 mmol/L, the renal threshold is reached and excessive urination occurs. This excessive urination is due to osmotic diuresis in which the concentration of glucose in the tubules of the kidney is so high it prevents the reabsorption of water (thus there is severe water loss). The body either has increased glucose in the blood, which can lead to heart failure, or it releases the glucose into the urine, which can lead to kidney failure. The kidneys, however take significantly longer to fail than the heart, so the body chooses the route that will give it the longest time to survive. In fact, blood glucose in the urine is one way of diagnosing a person with diabetes mellitus (hereinafter “diabetes”) since a person without diabetes will not show glucose in the urine. Thirst and frequent urination are symptoms of diabetes because the body is trying very hard to increase the intake of fluids, which help dispose of this excess, unwanted, dangerous glucose.

Even at 90% failure the kidneys will still operate and the person may feel quite normal. However, every 1% drop in kidney function thereafter will have the impact of losing 10% of remaining kidney function, and serious medical consequences result. Within several months of the kidney function dropping below 10%, kidney failure will occur.

The third of the above-described targets is the eyes. The eyes are filled with a dense fluid. When there are excessive amounts of glucose in the system, this fluid becomes denser, requiring more fluid to regain its optimum density. This extra fluid is forced into a space that has very few expansion possibilities. The result is that more pressure is exerted on the retina, leading to the condition known as glaucoma. At the same time, the arteries supplying blood to the eyes also become hardened, resulting in additional pressure at the back of the eye. Caught between these two pressures, the eyes suffer a variety of complications that damage the retina, and can eventually lead to total blindness. Other retinal disease, such as cataracts and retinal detachment, can also be brought on as a by-product of uncontrolled blood glucose levels.

The fourth of the above-described targets is the lower extremities, and particularly the feet. Many persons with poorly controlled blood glucose suffer from pain in the legs and feet, eventually losing feeling in the soles of their feet. This loss of feeling is due to the build-up of an insoluble substance called sorbitol, which collects inside the myelin sheath (insulation of nerves). After accumulating in the myelin sheath for some time, the myelin sheath is eventually ruptured, thereby causing the exposed nerve to stop functioning. The resultant loss of feeling makes walking very difficult, like walking with frozen feet. The attendant loss of feeling also makes it very difficult to feel any pain or discomfort.

One complicating factor of continuing high blood glucose is poor healing in general. High glucose concentration in the blood leads to poor circulation, which interferes with the natural healing process. This is one of the reasons why persons with poor control of their glucose have non-healing ulcers and are subject to increase incidences of infection.

Another complicating factor of continuing high blood glucose is diabetes. A person who does not have diabetes controls their blood glucose automatically and unconsciously, without being aware that it is even happening. Their blood glucose levels stay in the 4-7 mmol/L range, and any excess glucose is quickly removed and stored as glycogen or fat for future use. The key to this automatic regulation is the insulin produced by the pancreas. On the other hand, a person having diabetes who is dependent on insulin injections typically does not produce his or her own insulin, or produces insulin in insufficient quantities to maintain metabolic homeostasis, and therefore needs to take insulin via manual injections or an infusion device to overcome this deficiency. Those who do not monitor their blood glucose levels, at least via regular medical examinations, have no way of knowing their blood glucose levels, so they feel quite well with their blood glucose in the 12-14 mmol/L range or higher. However, with blood glucose at that level for several years, serious damage to the body can occur.

Diabetes can be classified in at least two ways, namely, as Type 1 diabetes or Type 2 diabetes. Persons suffering from Type 1 diabetes lose their ability to produce insulin, often because of an autoimmune response in which antibodies destroy the cells that make insulin, and become dependent on exogenous insulin injections or infusions to live. The amount of insulin required is based on several factors, the most important one being the amount of food that is eaten. As a result, diabetics have to be very careful about their food intake in order to control their blood glucose. Also many Type 1 patients utilize infusion pumps to provide both a basel level of insulin and a bolus injections of insulin.

Persons suffering from Type 2 diabetes lose (to varying degrees) the ability to promote glucose transport into the cells using insulin. A condition called “glucotoxicity” is brought about mainly by a diet of highly refined carbohydrates and poor exercise practices. It is rendered more severe by excess weight, but can be controlled by diet, exercise, medication, and sometimes by extra insulin.

Irrespective of diabetes or diabetic conditions, however, a person may become critically ill due to disease or as a result of accident-induced trauma. In critically ill patients, hyperglycemia and impaired metabolic function is prevalent, even in patients with no prior diabetes. Increased secretion of counter-regulatory hormones stimulates endogenous glucose production and increases effective insulin resistance. Studies also indicate that high glucose content nutritional regimes exacerbate hyperglycemia. Good glucose control has always been difficult to achieve in critically ill patients, and poor glucose control is directly related to infection, morbidity and mortality.

Hyperglycemia worsens outcomes, increasing the risk of severe infection, myocardial infarction, and critical illnesses such as polyneuropathy and multiple-organ failure. Significant reductions in other therapies with aggressive glycemic control may also be experienced. It also has a negative impact on neuromuscular complications and mechanical ventilator dependency. Hyperglycemia also affects the patients ability to facilitate natural healing process which lead to either new infections or deterioration of existing infections.

In critically ill patients, hyperglycemia and insulin resistance is often due to the physical and mental strains of the illness and the impact of any attendant drug therapy. Given the dynamics of the glucose-insulin systems, metabolic responses to stress of such patients are highly variable. Effects such as the increased secretion of counter-regulatory hormones may be realized, such effects leading to a rise in endogenously produced glucose as well as increases in the rates of hepatic gluconeogenesis and glycogenolysis. Typical glycemic control protocols designed for clinical implementation tend to reduce elevated blood glucose levels while accounting for inter-patient variability including size, age, and gender, conflicting therapies, and a dynamic metabolic system due to evolving patient condition. Effectively, the glycemic control protocols should be adaptive and/or able to identify changes in patient metabolic states, particularly with respect to primary indicators of metabolic state such as body temperature, renal function, blood pressure, urine output, medication dosage, liver function, and insulin resistance.

Insulin resistance is impaired biological response to either exogenous or endogenous insulin. Insulin sensitivity is a quantitative measure of insulin resistance and is a dynamic physiological parameter and a key driver of observed dynamics of the metabolic system for critical care patients. The level of a patient's insulin sensitivity is a snapshot of the current metabolic state of the individual. Insulin mediated glucose clearance is controlled primarily by insulin sensitivity which links insulin concentration and glucose levels. A low insulin sensitivity value is a likely indicator that a specific plasma concentration of insulin will not achieve a high rate of removal of glucose from the bloodstream. Insulin effect has also been shown to saturate in adult patients, thus additional exogenous insulin will have little or no effect on glucose levels once the saturation threshold is reached. This problem is exacerbated by the volatile metabolic condition of the intensive care patient.

On the other hand, the clinician is often faced with questions regarding how much insulin should be given for a future period of time, when the insulin rate should be reduced, and how the patient will react to the therapy based on their current metabolic state. Even though it is well accepted that tight glucose control saves lives, clinicians often encounter difficulties in delivering consistent therapy to patients. Furthermore, in hospital-type settings, there are often shift-to-shift differences in the administration of therapies to a single patient.

Glucose control is also a function of a patient's ability to burn calories. A patient's basal metabolic rate (BMR) can be responsible for burning up to about 70% of the total calories expended by a diabetic patient. However, the amount of calories burned by the BMR can vary substantially among diabetic patients or even over time in the same patient due to different factors. Factors that have an effect on the amount of calories burned by the BMR include, but are not limited to, the efficiency of respiratory function, the efficiency of the pumping of blood, and the maintenance of body temperature. Although the BMR is responsible for typically burning a substantial portion of the total calories expended by a hyperglycemic patient, it should be understood that the diabetic patient is capable of burning calories that are additional to those burned by the BMR.

Body temperature significantly affects human metabolism. Physiological effects of body temperature change on BMR in healthy individuals have been documented. The reduction of metabolic rate in relation to temperature espouses an exponential curve, with a greater delta at high temperatures (about 6% for 1 centigrade degree around 37 degrees centigrade) than at low temperatures (about 1% at 15 degrees centigrade). However, the link between metabolic state and insulin/glucose utilization has heretofore never been investigated. Critical care patients may suffer from any number of illnesses, including sepsis, which may induce severe fever. Large swings in body temperature result in considerable change in metabolic function. A general rule of thumb used in intensive care medicine is metabolic rate decreases by 6% every degree centigrade below normal body temperature and increases by 3% every degree centigrade above normal body temperature. Any recommendation system designed to achieve metabolic homeostasis should account for metabolic changes by swings in body temperature and titrate dosages to match the current metabolic demand.

Physical characteristics and body size including weight and height also play a role in the human metabolism particularly with respect to insulin clearance rates. Insulin clearance is defined as the plasma volume which can be purified of insulin in a time unit. An increase in insulin clearance occurs after loss of about 10% of initial body weight. A high degree in weight loss has also been correlated with decreases in insulin secretion. A high value of insulin resistance is a common feature in obese individuals, where the pancreatic β-cell sensitivity to increments in plasma glucose concentration is largely reduced compared to subjects with normal insulin sensitivity. Thus, insulin clearance and secretion rates are subject to inter-patient variability and patient specific parameters such as weight and height are often accounted for in metabolic dose management.

Additionally, metabolic function and in particular BMR are also dependent upon age and gender. A reduction in BMR with advancing age has been observed in a number of studies. After about 45 years of age, a progressive reduction in metabolic rate may be realized. This rate is related to a concomitant reduction in skeletal muscle mass. With regard to gender, it has been noted that women experience lower metabolic rates than do men, such rates being independent of differences in body composition and aerobic fitness.

Furthermore, it is well known that the kidney is the major site for insulin clearance from the systemic circulation, removing 50% of peripheral insulin and 50% of circulating pro-insulin. Any impairment in renal function limits the ability of the body to clear insulin, resulting in higher concentrations of insulin remaining in the blood stream. Clinically this is observed as an increase in insulin sensitivity and decreased insulin resistance. It is the lack of renal catabolism that is mainly responsible for this reduced insulin metabolism. Fortunately, clinicians measure the renal function of a hospitalized individual at routine and regular intervals via plasma concentrations of creatinine, urea, and electrolytes. Creatinine clearance is the most accurate measure and is used whenever renal disease is suspected. Creatinine clearance can be used to calculate the glomerular filtration rate (GFR), which is defined as the volume of fluid filtered from the renal glomerular capillaries into the Bowman's capsule (a cup-like sac in the kidney) per unit time, in an effort to assess renal function. Alternatively, an estimate of GFR can be calculated via the concentration of creatinine in the bloodstream and the Modification of Diet in Renal Disease (MDRD) equations. Studies have confirmed that hyperglycemia causes an increase in GFR.

Additional metrics related to renal function include aminoglyoside dosage and serum aminoglycoside concentration. From a clinical standpoint, it is clear that if over a two-hour period the renal function has significantly decreased, then less insulin will be cleared from the circulation system. Hence to avoid hypoglycemia the clinician should change the infusion of insulin to the patient. However the clinician is again faced with complex decision relating to how much the insulin dose should be varied by and for how long. One objective of the proposed invention is to provide the clinician the means to quickly and accurately incorporate renal function into the therapy decision process.

Type and severity of illness are additional drivers of impaired metabolic function, and the presence of the hyperdynamic state of sepsis leads to a decrease in glucose uptake and storage in comparison with healthy individuals. Clinical markers of sepsis including the presence of infection and severe inflammatory response (SIRS), multiple organ failure, fluid resuscitation, urine output, and inotrope dosage amounts are routinely measured in the intensive care unit. Decreased urine output is verified as a clinical indicator of sepsis. Hence if the clinician observes a drastic change in urine output and diagnoses sepsis they must quickly titrate insulin and nutrition dosages to counteract the impending rise in glucose levels.

The severity of sepsis and other illnesses can often be quantified using various methods. For example, APACHE II (“Acute Physiology and Chronic Health Evaluation II”) and SAPS II scores are measures of the severity of sepsis and other illnesses and are also important to assess metabolic state. The APACHE II is a severity of disease classification system and one of several ICU scoring systems. After admission of a patient to an intensive care unit, an integer score from 0 to 71 is computed based on several measurements; higher scores imply a more severe illness and a higher risk of death. Markers of the severity of illness must also be incorporated into the critical care metabolic management system. Blood pressure provides a snapshot of the current metabolic state and this data can be used in conjunction with other parameters to titrate insulin and nutrition dosages to enhance the healing process.

Blood pressure is also a precursor indicator of metabolic state and refers to the force exerted by circulating blood on the walls of blood vessels. The pressure of the circulating blood decreases as blood moves through arteries, arterioles, capillaries, and veins; the term “blood pressure” generally refers to arterial blood pressure, i.e., the pressure in the larger arteries. Typical values for a resting, healthy adult human are approximately 120 mm Hg systolic and 80 mm Hg diastolic (written as 120/80 mm Hg and spoken as “one twenty over eighty”), with large individual variations. These measures of blood pressure are not static, but undergo natural variations from one heartbeat to another and throughout the day (in a circadian rhythm); they also change in response to stress, nutritional factors, drugs, or disease. Blood pressure is but one indicator of insulin resistance and is easily and non-invasively measured at the patient's bedside. Blood pressure provides a snapshot of the current metabolic state and this data can be used to titrate insulin and nutrition dosages to enhance the healing process.

The states in which blood pressure is abnormally high or low are called hypertension and hypotension, respectively. Hyperinsulinemia (high concentrations of insulin in the blood) and/or insulin resistance has been linked to high blood pressure. One of the roles of insulin is to assist the storing of excess nutrients. Insulin also plays a role in storing magnesium. If the cells of a patient's body become resistant to insulin (insulin resistance increases), the body is unable to store magnesium and it is lost through urination. Intra-cellular magnesium relaxes muscles. When a patient cannot store magnesium because the cell is resistant, they lose magnesium and their blood vessels constrict. This causes an increase in blood pressure. Insulin sensitivity has been correlated to arterial hypertension or high blood pressure in the arteries.

In the ICU, patients may receive a cocktail of drugs, many of which are known and designed to have an effect on the human metabolism. One such drug is a catecholamine. Catecholamines are hormones released by the adrenal glands in situations of stress such as psychological stress or low blood glucose levels. Catecholamines cause general physiological changes that prepare the body for physical activity (“fight-or-flight” response). Some typical effects are increases in heart rate, blood pressure, blood glucose levels, and a general reaction of the sympathetic nervous system. Synthetic catecholamines are commonly used in intensive care as drugs. For example epinephrine or adrenaline is used medically to stimulate heartbeat and to treat emphysema, bronchitis, and bronchial asthma and other allergic conditions, as well as in the treatment of the eye disease glaucoma. Patients in intensive care generally undergo considerable stress and trauma and the secretion of cathecolamines from within the body is one of the main drivers of their high blood glucose levels. This is well known in the critical care profession and multiple studies confirm that stress-induced hyperglycemia involves increased catecholamines resulting in decreased effective insulin activity and decreased glucose utilization.

Synthetic catecholamines are administered to the patient to treat life-threatening conditions. These catecholamines further exacerbate the high blood glucose caused from the endogenous stress response. Thus, catecholamine dosage is another input to the complex metabolic system.

Pregnant patients in a critical care unit may also suffer from gestational diabetes. Gestational diabetes is a carbohydrate intolerance of variable severity that starts or is first recognized during pregnancy. Irregular menstrual cycles are a significant predictor of gestational diabetes mellitus.

Currently, clinical data relating to metabolic control obtained is poorly utilized by clinical workers through a lack of understanding of the dynamic changes and the complex interactions of the different measurements. This reflects the complex nature of physiology and modern clinical medicine. There exists a need for a simple device which can receive coded information relating to patient age, sex, body size, body temperature, renal function, blood pressure, urine output, medication history, and current glucose level and incorporate this information into the therapy decision.

Additionally, it is recognized by medical providers that critical care patients experience an induced “diabetic state” due to the stress and trauma of disease or illness. Many of the prior methods used for glucose control were originally developed for ambulatory Type 1 and Type 2 diabetics and do not take into account metabolic indicators such as body temperature, renal function, urine output, and blood pressure. In the majority of cases, these methods have significant limitations preventing them from being used in intensive care units. Initial work in metabolic control considered the body's blood glucose system a classic control system, consisting of a process in which the aim is to maintain control over an output (blood glucose) in the presence of zero or non-zero inputs (food, physical exercise, insulin) and in the presence of perturbations (emotional status, illness).

This output is achieved by feedback, namely, continuously measuring the output values, comparing these points to the desired state (set point), and initiating compensatory action. Such methods are exemplified by U.S. Patent Application 20070048691 to Brown which presents a diabetes self-care system comprising a blood glucose meter linked to a micro-processor. The micro-processor based unit sends a signal to inject insulin when the blood glucose level exceeds a predetermined range. This is an example of a single input single output control system that is replete in the literature. This closed loop system is driven solely by blood glucose measurement and hence utilizes a test-fix-test approach. Similarly, U.S. Patent Application No. 20060264895 to Flanders presents a system for managing glucose levels in patients with diabetes or hyperglycemia. The system uses a target range of blood glucose and recommends an insulin dosage when the measured blood glucose exceeds the upper range limit and a glucose dose when the measured blood glucose is below the lower level of the range. The system fails to take into account any additional dynamics not captured or inferred from blood glucose measurement. Alternative approaches are needed that allow prevention-driven proactive care, providing information when the clinician needs it, not after the fact. The test-fix-test approach of Brown and Flanders is neither simple nor easy for the healthcare worker to implement. Proactive control is more desirable than a test-fix-test approach and to address this numerous techniques for predicting blood glucose measurements have been devised.

Predictive methods have also been employed. For example, U.S. Pat. No. 5,840,020 to Heinonen et al. addresses a method for predicting the glucose level in a patient's bloods utilizing an adaptive model which utilizes data on the patient diet, medication, physical strain, and actual blood glucose measurements. The error between a predicted value and the actually measured value is used to optimize (converge) a dynamical model in a recursive fashion using Widrow's Adaptive Least Means Square algorithm. In this patent, Heinonen utilizes a limited data set of inputs similar to Brown and Flanders. Heinonen changes the mathematical model in each feedback loop resulting in considerable computational effort to revise and update the model.

U.S. Pat. No. 6,421,633 to Heinonen discloses a mathematical model which predicts future glycosylated haemoglobin (HbA1C) behavior from previously measured HbA1C levels and blood glucose levels. U.S. Pat. No. 7,025,425 to Kovatchev et al. discloses a computer program capable of predicting glycosylated hemoglobin (HbA1C) and the risk of severe hypoglycemia. These predictions are based on blood glucose readings collected by a self-monitoring blood glucose device. HbA1C is an indication of long-term metabolic control with limited benefit in guiding therapy in the volatile patient environment of a critical care unit.

U.S. Patent Application No. 20060025931 to Rosen teaches methods for predictive modeling of patient condition utilizing time-stamped data of physiological measurements and advanced statistical methods such as variance detection algorithms. This data-driven approach uses purely statistical descriptions of the data and hence can only provide implicit correspondence to the underlying physiology and gives a limited understanding of the actual mechanics involved. The method uses inputs of blood glucose level, blood pressure, blood oxygen saturation, electrical activity, weight, and physical activity to predict the future condition of the patient. Rosen does not account for body temperature, renal function, or body mass index among others. More importantly the method does not use the predictions in an on-going manner to guide therapy or titrate dosages and hence is more an education tool and an assistive device. U.S. Pat. No. 6,272,480 to Tresp utilizes neural modeling of the dynamic metabolic system trained with the assistance of an adaptation rule that makes use of the forward or backward Kalman filter equations. The model is utilized in order to predict values of glucose-insulin metabolism of a diabetic patient. Neural techniques provide effective models to handle large data sets but neural models do not accurately describe the complex interactions of the underlying metabolic physiology. The methods of Rosen and Tresp do not employ a physiological accurate model of the underlying laws governing metabolic behavior; hence the effectiveness of new methods and therapies is limited.

U.S. Pat. No. 6,923,763 to Kovatchev discloses a method which utilizes blood glucose (“BG”) sampling, insulin infusion/injection records, heart rate (“HR”) information, heart rate variability (“HRV”), and electrocardiogram (“EKG”) information to estimate blood glucose in the near future and to estimate the risk of the onset of hypoglycemia. This invention requires complex communication means to connect a processing system to physiological sensors measuring HR, HRV, and EKG or require an intense data input effort from clinicians. The system requires extensive modification to the existing bedside monitoring systems. In addition the device requires a complex interface for inputting or reviewing the HR, HRV, and EKG patient data. Ease-of-use to nursing staff is a core requirement of any protocol designed for clinical implementation and this requirement is not addressed by the system. A need exists for a simple and portable system with minimal data input and clinical effort that could be easily integrated to current practices in any ICU.

U.S. Pat. No. 6,572,545 to Knobbe discloses a method for estimating the blood glucose level in real time from an alternative signal that is a function of the glucose level using a linearized Kalman filter. Such estimation techniques inevitably increase measurement error and increase the risk of missing swift changes in blood glucose characteristic of patients in critical care.

U.S. Patent Application 20070012324 to Nirkondar teaches a handheld device for diabetes management. This device contains nutritional information on up to 35,000 food items and allows the user to easily store information on food eat, glucose levels, medication, insulin and exercise data. The device is a suitable method for organizing and maintaining data but is not capable of using the data to generate recommendations or titrate therapy.

U.S. Pat. No. 6,925,393 to Kalatz teaches a system for extrapolating a future glucose concentration from inputs of administered insulin doses, carbohydrates consumed or planned, time of administration, and blood glucose measurements. U.S. Pat. No. 6,835,175 to Porumbescu addresses a predictive device which generates ongoing metabolic state predictions from user-patient inputs. This device allows users to accept or reject time-stamped inputs used in the metabolic state prediction. A commercial system, KADIS, is in use in Germany as a model-aided education tool for insulin dependant diabetes mellitus (IDDM) patients. The system provides tools for the retrospective analysis of data resulting from home blood glucose monitoring.

U.S. Pat. No. 7,167,818 to Brown teaches a disease simulation system and method capable of simulating and displaying a future blood glucose value based on previous blood glucose measurements, optimal self-care values, and one or more scaling factors. U.S. Patent Application Number 20060272652 to Stocker explains a virtual patient software system for educating and treating individuals with diabetes. The system allows individuals to develop their own therapy routine by providing a simulation engine capable of generating and displaying blood glucose predictions based on patient inputs such as planned carbohydrate intake. What the methods of Kalatz, Porumbescu, Brown, and Stocker and the systems of Kadis have in common is the lack of capability to process the information and utilize the predictions in an on-going manner for redirecting glucose levels in a real-time environment. For this reason they are more in the nature of educational tools, than of assistive devices which can be used in a clinical setting. While providing predictive values of a glucose level, they do not recommend or provide guidance in selecting therapy or dosage requirements. To provide guidance methods closed loop devices and assistive methods have been developed to help overcome the issues of existing predictive models.

Assistive technologies designed to provide guidance to diabetic individuals to control their dynamic and impaired metabolic state and related methods are also known in the art. U.S. Patent Application No. 20060089540 to Meissner describes a device for diabetes management utilizing a plurality of alarms to remind the patient when a dose of food or medicine should be taken. The device aids the individual in ensuring the chosen dosages are administered but does not provide guidance in titrating dosage amounts.

U.S. Pat. No. 7,022,072 to Fox teaches a system for monitoring physiological characteristics and, in the case of diabetes, anticipating harmful conditions such as a glucose crash or impending hyperglycemia. The blood glucose sensor in this system is linked to a processor for automated data transfer. Automated and “continuous” glucose sensors with an acceptable level of error for intensive care application are not yet available on the market. Any system designed for widespread hospital use must be compatible with the large variety of existing blood glucose measurement techniques currently employed in ICUs. The methods of Fox utilize blood glucose measurement but do not account for additional metabolic factors including renal function, body temperature, and body mass index.

U.S. Patent Application No. 20060122099 to Aoki teaches a method for infusing insulin to a subject to improve total body tissue glucose processing. The method involves ingesting a carbohydrate containing meal followed by the delivery of a series of pulses of insulin over a period of time. The number of pulses, the amount of insulin in each pulse, the interval between pulses and the amount of time to deliver each pulse to the subject are varied until the subject's total body tissue processing of glucose is restored. In essence this device calculates insulin dosage recommendations from blood glucose measurements and fails to incorporate any additional metabolic factors.

U.S. Pat. No. 5,997,475 to Bortz teaches a device management system using a programmable microprocessor based unit having a display, keyboard, and memory. The microprocessor is programmed to determine a recommended amount of insulin based upon the carbohydrates ingested, a glycemic index of the carbohydrates, the activity status of the user, and current blood glucose levels. U.S. Pat. No. 5,216,597 to Beckers discloses a system and apparatus for efficient control of diabetes comprising a recorder, an interface and a computer. The computer means is programmed to develop the optimum program of treatment including insulin medication, diet, and exercise for a patient on the basis of inputs of blood glucose, food intake, insulin dosage and exercise. Such a device is an example of the traditional approach taken to glucose control where the key control input variables accounted for are insulin, nutrition, exercise, and blood glucose measurements. Such systems do not provide adequate control because they do not account for precursory metabolic indicators such as body temperature, renal function, and urine output. U.S. Pat. No. 5,420,108 to Shohet is a method for controlling diabetes mellitus which utilizes blood glucose measurements, urine sugar test results, insulin and sugar dosages to titrate sugar and insulin dose required to provide tight control. U.S. Pat. No. 7,137,951 to Pilarski teaches a method of food and insulin dose management for a diabetic subject utilizing inputs of intended insulin unit value or an intended carbohydrate unit, blood glucose measurements, a target blood glucose range, and time schedule of planned dosages. These inputs are used to determine a balance value of either insulin units or carbohydrate units needed to balance with the provided values to maintain blood sugar in the subject in a target blood sugar range.

Additionally, U.S. Pat. No. 7,179,226 to Crothall discloses a system and method used to manage the blood glucose of a diabetic individual. Crothall teaches a computer implemented method on a computer readable medium providing the patient with a data input interface allowing the patient to: i) enter time and intensity of physical activity performed ii) enter time and value of blood glucose measurement data iii) enter time and amount of food intake iv) enter time and value of insulin intake history. These inputs are used to calculate insulin and carbohydrate intake recommendations. Crothall ignores the other critical physiological inputs need to control glucose levels in critically ill patients. U.S. Pat. No. 4,731,726 to Allen III relates to a home monitoring system including a computer assisted reflectance photometer designed for measuring blood glucose values at home, and for storing and transmitting these and other data to a physician in connection with administration of treatment for diabetes mellitus. The monitoring system uses inputs of patient data relating to diet, exercise, emotional stress, and symptoms of hypoglycemia, including fever, to generate a recommended supplemental insulin dosage.

The patents to Bortz, Beckers, Shohet, Pilarski, Crothall, and Allen III all use combinations of the traditional inputs of insulin dosage, nutritional dosage, blood glucose measurement, and exercise history. Numerous studies provide evidence that additional patient-specific physiological parameters play important roles in human metabolism. These existing systems do not take into account renal function, body temperature, urine output, or physical characteristics such as gender, age, and body mass index in titrating individual therapy. There exists a need for an easily implemented system that accounts for these additional patient-specific metabolic indicators among others.

An alternative to traditional systems is U.S. Pat. No. 6,572,542 to Houben that teaches a system utilizing information derived from an electrocardiogram (‘ECG’) and electroencephalography (‘EEG’) signals to predict the onset, or indicate the presence of hypoglycaemia in a human patient. Detection of a hypoglycemic event by the system activates an alarm or commences the delivery of a beneficial agent such as insulin, glucagon or diazoxide to the patient. ECG signals record the electrical activity of the heart while EEG is the neurophysiologic measurement of the electrical activity of the brain. ECG and EEG signals are accurate measures of cardiac and nervous system activity. However using such a device would require critical care patients to be continuously monitored on both an ECG and EEG. The data signals would also need to be transferred to a control system requiring additional network capability and infrastructure costs. There exists a need for a system which can be seamlessly integrated with existing systems and methods currently employed by ICUs and that nurses are accustomed to using.

Body temperature is addressed as an indicator for blood glucose levels in both U.S. Pat. No. 6,882,940 to Potts and U.S. Pat. No. 6,233,471 to Berner. Potts describes a method, device, and microprocessor for predicting and alerting a user of a hypoglycemic event. This device utilizes inputs of frequently obtained glucose values, body temperature, and skin conductance. This invention is designed to detect a hypoglycemic event when an individual is sleeping or unable to regularly measure their blood glucose. Hypoglycaemia is a part of the challenge in critical care but there is a need for a protocol that incorporates body temperature that not only prevents hypoglycaemia but aggressively minimizes hyperglycaemia as well. Berner teaches a method for continuously measuring the blood glucose by processing and filtering alternative signals that are closely correlated with the blood. The method uses measurements of body temperature, perspiration levels, and skin conductivity to correct and calibrate a signal indicative of blood glucose level. The methods of Berner and Potts introduce new sensors capable of recording and using body temperature, among other variables, as a surrogate signal to infer actual blood glucose concentration. Novel sensors may be of use for ambulatory diabetics but ICUs already possess extensive equipment capable of measuring temperature and blood glucose. In addition Berner and Potts work are only indicators of blood glucose levels and do not address the significance to the body of temperature as an indicator to insulin and glucose utilization. They also do not use temperature to titrate insulin and nutritional dose, hence they are measurement and prediction devices as opposed to a purely assistive device. There exists a need for a cost effective simple device that can guide therapy, based on precursory metabolic indicators such as renal function and urine output among others, and be used in synergy with existing measurement techniques.

U.S. Pat. No. 6,602,715 to Yatscoff teaches a novel system utilizing multiple breath tests and blood glucose measurements prior and following the ingestion of an enriched glucose source to diagnose diabetic indications and monitor glycemic control. Breath analysis is a novel advancement that could eliminate existing painful finger pricking methods. The breath test invention is simply used as a surrogate measurement of blood glucose. The methods of Yatscoff propose a new sensor but do not use the sensor to guide therapy.

U.S. Patent Application No. 20020107476 to Mann teaches a typical insulin pump that attaches to the body and is capable of infusing liquid based on remote commands. Such infusion pumps are designed for ambulatory diabetics and not applicable for wide-spread use in intensive care units.

U.S. Pat. No. 6,544,212 to Galley teaches a diabetes management system including an insulin delivery unit, a glucose sensor, and a control unit. The system is capable of predicting glucose values at a predetermined time in the future and the control system disclosed automates the processes of glucose measurement and insulin delivery. However Galley's automated glucose measurements used in the algorithm do not account for the varied physiological factors that affect glucose levels and can result in significant error.

U.S. Pat. No. 7,060,059 to Keith addresses a method and system for controlling the concentration of a substance in a patient using a model-based controller with an intra-dermal (ID) delivery device. U.S. Patent Application No. 20030130616 to Steil et al. addresses a closed-loop method and controller for infusing insulin to a user. The controller commands are driven by a proportional plus, integral plus, or derivative (PID) controller and the liquid infusion is based on this command. U.S. Patent Application No. 20020040208 to Flaherty et al. is an additional closed loop system designed to deliver a liquid infusion into a patient based on a measured physiological parameter. U.S. Patent Application No. 20050171503 to Van den Berghe teaches an automatic infusion system linked to a sensor indicative of a patient's blood glucose level. These systems do not address the varied physiological factors that affect glucose levels such as renal function, body temperature, and body mass index. Whereas these closed loop systems may work well for ambulatory patients with an impaired metabolic state, they typically have high costs and logistic barriers to wide-spread implementation in intensive care units. In addition current automated “continuous” sensors used in these closed loop systems still face significant issues related to error, ease of use, and reliability.

U.S. Pat. No. 5,364,346 to Schrezenmeir teaches a process for the continuous and discontinuous administration of insulin to the human body. An optimal insulin dosage is titrated from an insulin challenge followed by a blood glucose test. This method is appropriate for determining a substitute basal insulin rate in ambulatory diabetics but not applicable to on-going dosage calculations as required in an ICU.

U.S. Pat. No. 6,740,072 to Starkweather teaches a system and method for providing a closed loop insulin infusion system. The parameters of the sensed biological state, in this case blood glucose, are measured and uploaded at timed intervals and specific time periods. This invention is limited by the fact that it can only infuse insulin. U.S. Pat. No. 6,379,301 to Worthington discloses a diabetes management system and method for controlling diabetes. This apparatus, intended for patients with diabetes mellitus, predicts a future blood glucose value from inputs of blood glucose, previous insulin dose, and a provided insulin sensitivity value. The device outputs a corrective action bolus calculated to achieve a predictive blood glucose value in a target blood glucose range. Worthington and Starkweather do not address the nutritional intake of the patient which is a proven driver of metabolic balance. Modulating the nutritional intake offers a means of reducing glycemic levels in the face of significant insulin resistance, as seen in the very critically ill.

Generic hospital treatment systems are also in use. U.S. Patent Application No. 20050159987 to Rosenfeld teaches a system and method utilizing a datastore for standardizing care in a hospital environment. The invention is designed to provide a system and method for remote monitoring of ICUs from a distant command center/remote location to provide 24-hour/7-days-per-week patient monitoring. The disclosure is capable of providing monitoring and decision support for over 140 conditions. Providing therapy guidance for such a large number of diseases requires significant computing power and a large database of clinical markers and symptoms to correctly diagnose each condition. The first step of this invention involves the input of patient data to identify an appropriate decision support algorithm to implement. This requires additional time on an already strained clinical work force. There exists a need for a system designed specifically for metabolic management that can be used locally by ICU healthcare workers quickly and effectively every 1-2 hours and also incorporates key parameters including body temperature and renal function among others.

U.S. Patent Application No. 20060253296 to Liisberg discloses a medical advisory system. The invention is capable of providing recommendations to a clinician or patient that are generated via processing means that utilize a plurality of mathematical advisory models. Multiple models are beneficial in treating and generating recommendations for numerous conditions however because of the multiple models the complexity of the system is increased the ability to implement it in the ICU is reduced because of cost, numerous inputs required and complexity. However, there exists a need for a system designed specifically for metabolic management that can reduce a complex multi input/output system into a single model and simple device that will adjust for the various complex responses of the metabolism of the patient.

U.S. Patent Application 20060111933 to Wheeler teaches an adaptive medical decision support system for providing diagnostic and treatment information to a health professional. The invention utilizes a database of diagnosis and treatment rules networked to records containing patient-specific data. Such a system requires substantial integration with existing data records and would have significant cost and logistical barriers to wide-spread implementation. There exists a need for a simple semi-closed loop system which can be easily implemented and efficiently used by clinicians without extensive information technology system integration.

Manual calculation devices have been used in the healthcare field. For instance, U.S. Pat. No. 4,308,450 to Ausman teaches a two-piece slide calculator for determining basal energy expenditure, body surface area, ideal body weight, and carbohydrate dosage for a parenteral feeding solution.

U.S. Pat. No. 6,543,682 to Glaser and U.S. Pat. No. 6,691,043 to Ribeiro present methods for generating insulin dosage recommendations. Glaser teaches a manual circular calculator for determining an appropriate insulin injection dosage to be taken with a meal. The device utilizes inputs of current blood glucose measurement and planned carbohydrate amount to titrate an insulin dose. This methodology does not address the other metabolic factors that contribute to glucose levels in the body. The primary control input to control blood glucose in intensive care is the administration of exogenous insulin. However, since the insulin effect saturates around 5-10 U/h in continuous infusion, modulating the nutritional input offers a means of reducing glucose levels, in the face of significant insulin resistance, as seen in the very critically ill. Thus, this device is not designed for use in the ICU where insulin alone may not be able to fully achieve the desired glycaemic reductions. Ribeiro teaches a bolus calculator which faces the same limitations in an ICU. There exists a need for a simple, low-cost device designed specifically for the ICU that can provide dosage recommendations for both nutrition and insulin and incorporates all the relevant clinical datapoints.

Additional manual calculation devices are used in the health-care field. U.S. Pat. No. 4,149,068 shows a circular slide rule improvement which is said to be used in particular for use in X-ray dosage calculations. U.S. Pat. No. 4,882,472 to Sigman teaches a circular calculation device for determining fluoride supplement dosages. U.S. Pat. No. 5,640,774 to Goldman teaches a circular calculation device to calculate caloric quantities of food.

The disclosures of the above patent specifications are incorporated herein by reference in their entirety. The assistive technologies discussed herein are designed to aid ambulatory individuals control a dynamic and impaired metabolic state—there is a need for a simple manual-based system designed specifically for the practicalities of critically ill patients and their clinicians. Given the volatile nature of the metabolism of a critically ill patient there exists a need for a system that can not only track the underlying metabolic state but also accurately match therapeutic insulin and nutritional inputs with the ability to utilize these inputs. In a critical care environment where one clinician may be treating multiple patients with impaired metabolic ability, a methodology and decision assist system can be of obvious benefit.

Currently, no intensive insulin therapy protocol utilizes variability or changes in insulin sensitivity or metabolic state to guide therapy or intervention for a future period of time, leaving clinicians partly blind in controlling such a highly dynamic system. The focus has been on responding to glucose levels with insulin which is a single input and single control variable system. This single data point and single control point methodology is not capable of controlling a highly complex multivariable system such as mammalian glucose levels. Previous methods for determining insulin sensitivity have been computationally burdensome and laborious with regard to time.

In view of the foregoing, what is needed is a convenient and easily applied method for monitoring the metabolic parameters of critically ill patients and patients undergoing intensive care.

There is also a need to increase the availability and improve the form of information to the clinical decision maker, lessening their uncertainty and improving the quality of care delivered.

There is also a need for a system that accounts for age and gender to provide customized patient-specific metabolic control treatment.

There is also a need for a system which can accurately account for the relevant metabolic inputs, including catecholamine dosage, and quickly generate therapy recommendations to aid the clinician in delivering a high level of care.

There is also a need for a repeatable process and device that can assist clinicians in reducing a patient's blood glucose levels in a predicted and controlled manner.

There is also a need for a device that can alleviate the burden places on the caregiver in an ICU of having to operate several devices (e.g., nutrition delivery, insulin delivery, and body fluid analyte sensors) independently by intensive care nursing staff by automatically linking a glucose sensing step with an insulin and nutrition delivery system.

There is also a need for a multivariable system that can solve complex problems relating to the control of blood glucose levels.

There is also a need for a novel method of accurate and quick identification of patient insulin sensitivity and current metabolic state to be part of any metabolic protocol in the highly variable critical care population. Such an adaptive protocol would allow clinicians to anticipate and identify changes in patient dynamics and match therapeutic inputs to each individual patient's needs.

There is also a need to reduce the complexity of the systems presently in use in which multiple zero- and non-zero inputs and outputs are used to track metabolic functions.

Given the large number of inputs to the complex metabolic system of a critically ill patient, what is also needed is a clinical device that can aid the clinician in processing information.

There is also a need to lessen the uncertainty of decisions, and improving consistency and quality of care by providing consistent therapy recommendations.

There is also a need for a clinical device that can be used to better aggregate measurements and create an accurate picture in an effort to enhance diagnostic capability and improve treatment selection.

There is also a need to provide a management device for gestational diabetes that incorporates menstrual cycle data and additional data relating to pregnancy including, but not limited to, due date and stage in pregnancy.

There is also a need to provide a method of increasing the effectiveness of ventilation therapy by controlling the healing process by controlling glucose levels in a patient.

There exists a need to digitize existing analog computation devices to improve usability and enable, for example, electronic data transfer.

There exists a need for an electronic device that incorporates metabolic, nutrition and glucose levels with markedly enhanced convenience from paper-based metabolic control systems.

There exists a need for a simple electronic user interface to promote product reliability and clinical staff compliance.

There exists a need for a device that can automatically customize a nutrition algorithm, designed to achieve tight glucose control, to match the nutrition practices employed at different ICUs.

There exists a need for a device that will allow the clinician to customize nutrition and insulin algorithms to a target glycemic band and measurement frequency selected on a per-patient basis.

There exists a need for an electronic system that will promote better control of critically ill patient glucose levels, ultimately providing the proven medical benefits of tight glycemic control in acute care settings.

There exists a need for an electronic system that accounts for indicators of the patient metabolic state, can generate insulin and nutrition dosage recommendations, request confirmation/approval of dosages by nursing staff and then communicate the dosages to existing critical care fluid pumps.

There also exists a need for an electronic system that is in communication with temperature sensors and blood glucose sensors, account for indicators of the patient metabolic state, generate insulin and nutrition dosage recommendations, request confirmation/approval of dosages by nursing staff and then communicate the dosages to existing critical care fluid pumps.

### SUMMARY OF THE INVENTION

Disclosed herein are computational devices, methods and systems that employ clinical measurements relevant to metabolic functions for determining various dosing recommendations for a future period of time, reducing dependency on mechanical ventilation processes, and/or improving ventilation therapy in order to obtain and maintain metabolic stability in critically ill and/or diabetic patients. The devices utilize patient specific metabolic information and current conditions to ensure that patients are administered consistent and controlled therapies during treatment periods. Although the devices, methods, and systems of the present invention are generally employed during the administering of intensive care treatments, the present invention is not limited in this regard and the devices, methods, and systems can be used in conjunction with other treatments or therapies.

The computational devices may be either digital or analog. The digital computational devices are generally software based devices that are used for determining patient specific nutrition and insulin dosages from inputs such as renal function, age, gender, weight, height, body temperature, insulin history, nutrition history, and blood glucose measurements. The analog devices are generally slide rule-type devices that are used for determining patient specific nutrition and insulin dosages from similar inputs.

Irrespective of whether the computational devices are digital or analog, the patient specific nutrition and insulin dosages are administered to critically ill patients to stabilize their glucose levels in a preferred 4-6 mmol/L range or in another desirable range.

One particular metabolic indicator that is of particular relevance to the critically ill and/or diabetic patient is body temperature. Accordingly, the present invention described herein can utilize body temperature to provide a link between the metabolic state of such a patient and the insulin/glucose utilization mechanisms of that patient. For every increase of 1 centigrade degrees in internal body temperature, the BMR of the patient increases by about 6%. Chemical reactions in the body (e.g., the reaction of carbohydrates into glucoses and the like) occur more quickly at higher temperatures. Therefore, a patient having a fever of 42 degrees C. (about 4 centigrade degrees above normal) would have an increase in BMR of about 24%. However, temperatures outside of the normal ranges, whether higher or lower, sabotage the body's ability to utilize insulin, and insulin resistance is increased.

Any system that is targeted at helping control glucose and insulin levels in a critically ill or diabetic patient preferably utilizes the overall health of the patient as predicted by the BMR and insulin sensitivity as a critical driver for the glucose-insulin regulatory system. Therefore, one aspect of the invention is the generation of knowledge-based therapy recommendations. For metabolic decision support therapy recommendations using the BMR and insulin sensitivity, these outputs can include information derived from dosage amounts and dose administration times of insulin, insulin analog, or insulin mimetic; dosage amounts and dose administration times of nutrition including carbohydrates, proteins, lipids, fats, and sugars; time and intensity recommendations for exercise and physical activity; information pertaining to drug therapy selection, decisions to stop or continue ventilation therapy, and decisions to perform or stop alternative clinical intervention; dosage amounts and times of administration for antibiotics, cardiac medicines, prokinetics, steroids, sedatives, vasoactive drugs, or any other medication; diagnoses of conditions and information pertaining to the tracking of patient conditions; patient body temperature; decisions to deliver or perform methods of treatment; selection of laboratory protocol; medication dosages such as blood pressure reducing medications, aspirin and the statin cholesterol-lowering drugs, blood replacement fluid dosages, colloid and crystalline fluid dosages, vasoactive drug dosages, cathecolamine dosages, synthetic cathelamine dosages, phosphodiesterase inhibitor (PDI) dosages such as Amrinone, Milrinone, Enoximone and Piroximone, surgery or angioplasty; aminoglycoside dosages, noradrenaline dosages, dosage of muscle relaxants usage such as pancuronium, rocuronium, dosage for any known pharmaceutical which is at least partially cleared by the kidney; decisions or times to retest blood glucose value or alternative physiological measurement; authorizations to subject the hospitalized patient to a diagnostic procedure selected from the group consisting of laboratory protocols, ventilator protocols, hemodynamic protocols, and radiology tests; authorizations to subject the hospitalized patient to treatment procedures selected from the group consisting of radiological procedures and a surgical procedure; and authorizations to administer medications to the hospitalized patient. The selection of laboratory protocol includes, but is not limited to, adjustment of environment settings for patients in isolation, central venous catheter line change, air viva/laerdel bag assembly and cleaning, central venous catheter port designation, central venous catheter site dressing, cleaning of electrical equipment contaminated with body products, cooling of patient, drawing blood cultures, drawing blood from a central line, drawing blood from a central venous catheter, drawing blood samples from radial/femoral arterial lines, dressing changes to radial/femoral arterial lines, emergency defibrillation, emergency intubation, enteral feeding, the use of heparin locks for central venous catheter lines, changes to humidifier settings, insertion or removal of an arterial line, insertion or removal of nasogastric tube, intra-abdominal pressure monitoring, monitoring of alarm parameters, continuous positive airway pressure oxygen therapy (CPAP), ventilatory assistance, plasmapheresis, pulmonary capillary wedge pressure measure, pulse oximetry, securing and care of endotracheal tubes, suctioning a patient with a tracheostomy tube, suctioning a patient with an endotrachael tube, total parental nutrition administration, transfer of patient to other ward areas, ventilator emergency, and ventilator circuit set-up and assembly. The present invention is not limited in this regard, as other recommendations and outputs are possible.

In one aspect, the present invention resides in a method of providing blood glucose therapy for a critically ill patient. This method employs calculating a baseline nutrition feed requirement for the patient based on an algorithm that incorporates at least one of the patient's age, gender, and body frame size: determining a first blood glucose level of the patient; determining at least a second blood glucose level of the patient after a preselected time interval: determining a first body temperature reading of the patient, comparing the first and second blood glucose levels: and administering either nutrition or insulin. The amount of nutritional feed administered to the patient is based on a first change in blood glucose level, the current body temperature reading, and a predetermined feed algorithm based on the second blood glucose level as well as the patient's baseline nutritional feed requirement. The amount of insulin administered to the patient is based on a second change in blood glucose level, the current temperature, and a predetermined insulin algorithm that incorporates at least one of the patient's body frame, age, or gender. A blood glucose measurement frequency recommendation may also be calculated from the current blood glucose level and the change in blood glucose level.

In another aspect, the present invention resides in a method of determining a nutritional input and an insulin input for a discrete time period for a critically ill patient. This method includes determining an insulin scaling factor; determining a nutrition scaling factor; determining a precursor of a metabolic state of the patient based on a selected metabolic marker; determining a blood glucose level of the patient; entering data indicative of the scaling factors and precursor of the patient as well as the blood glucose level of the patient into an electronic calculation device; and calculating insulin and nutrition amounts to be administered to the patient. The insulin and nutrition amounts are based on the entered information.

In another aspect, the present invention resides in a digital computational device for assisting a clinician in determining a therapy for a patient. This device calculates a recommended nutrition rate from a first corresponding algorithm and a recommended insulin dosage from a second corresponding algorithm. The first corresponding algorithm includes calculating a first value from one or more physiological parameters specific to the patient and one or more real-time precursor status indicators. The second corresponding algorithm includes calculating a second value from one or more physiological parameters specific to the patient and one or more real-time precursor status indicators. The calculated recommended nutrition rate and the calculated recommended insulin dosage are incorporated into the therapy for obtaining and maintaining metabolic homeostasis in the patient.

In another aspect, the present invention resides in a method of establishing metabolic homeostasis in a patient having hyperglycemic blood glucose levels. This method includes the steps of inputting three physiological parameters into an electronic calculation device. The first physiological parameter is a factor specific to the patient; the second physiological parameter is a real-time parameter comprising a factor indicative of the patient's metabolism; and the third physiological parameter includes factors derived from current and past data relating to the patient. A recommended dosing rate to be administered to the patient is then calculated from the parameters.

In another aspect, the present invention resides in a method of using a digital computational device to establish metabolic homeostasis in a patient having a hyperglycemic blood glucose level. This method includes the steps of inputting information specific to the patient into the digital computational device, the information being indicative of at least one of the age, gender, and body size of the patient; inputting a physiological parameter into the digital computational device, the physiological parameter comprising a real-time parameter comprising a factor indicative of the patient's metabolism; inputting a blood glucose value of the patient and prior nutrition and insulin dosages delivered to the patient relating to a discrete time period into the digital computational device; and calculating a recommended dosing rate to be administered to the patient, the recommended dosing rate being calculated from the input information specific to the patient, the input physiological parameter, and the input blood glucose value and the prior nutrition and insulin dosages. A recommended blood glucose measurement frequency for the patient may also be calculated, the measurement frequency calculated from past and present blood glucose value.

One advantage of the devices, methods, and systems of the present invention is that tight glucose control to limits of 4-6 mmol/L can reduce ICU patient mortality between 18-45% (relative) for patients with greater than a 3 day stay in the ICU.

Other advantages of the devices, methods, and systems of the present invention will become apparent from the following detailed description.

### BRIEF DESCRIPTION OF THE DRAWINGS

FIG. 1 is a perspective view of a computer of the present invention used to calculate nutrition and insulin dosage recommendations.

FIG. 2 is a flow chart of the recommended method of using the device of FIG. 1 to determine a recommended nutrition and insulin dosage for a critically ill and/or diabetic patient.

FIG. 3 is a schematic diagram of the structure of the software program for one embodiment of the present invention.

FIG. 4 is a schematic diagram of the operations that perform the set-up of a new patient routine for a one embodiment of the present invention.

FIG. 5 is a schematic diagram of a method used to determine insulin, nutrition, and measurement frequency recommendations during each iteration of a control program of the present invention.

FIG. 6 is a report for the clinical provider showing recommended insulin and nutrition.

FIG. 7 is a nutrition table, of the present invention, presented via a computer graphic.

FIG. 8 is an insulin table, of the present invention, presented via a computer graphic.

FIG. 9 is a schematic diagram of data input and output interfaces incorporated in one embodiment of the present invention.

FIG. 10 is a front perspective view of a slide rule computational device of the present invention.

FIG. 11 is a back perspective view of the computational device of FIG. 10.

FIG. 12 is a plan view of the front of the computational device of FIG. 10 with a slide member in place.

FIG. 13 is a plan view of the back of the computational device of FIG. 10 with the slide member in place.

FIG. 14 is a plan view of the front of the slide member.

FIG. 15 is a plan view of the back of the slide member.

FIG. 16 is a flowchart illustrating a process of determining a recommended nutrition dosage.

FIG. 17 is a flowchart illustrating a process of determining a recommended insulin dosage.

FIG. 18 is a first sheet of a flow chart, of the present invention, for use in determining insulin and nutrition dosages.

FIG. 19 is a second sheet of the flow chart, of the present invention, for use in determining insulin and nutrition dosages.

FIG. 20 is a third sheet of the flow chart, of the present invention, for use in determining insulin and nutrition dosages.

FIG. 21 is a fourth sheet of the flow chart, of the present invention, for use in determining insulin and nutrition dosages.

FIG. 22 is nutrition table, of the present invention, for use in determining nutrition dosages.

FIG. 23 is an insulin table, of the present invention, for use in determining insulin dosages.

FIG. 24 is a front perspective view of a computational device in the form of a circular slide rule of the present invention.

FIG. 25 is a back perspective view of the analog computational device in the form of a circular slide rule of the present invention.

FIG. 26 is a plan view of the circular slide rule of FIG. 24 showing instructional text and calculating information printed on a front panel thereof.

FIG. 27 is a plan view of the circular slide rule of FIG. 24 showing instructional text and calculating information printed on a back panel thereof.

FIG. 28 is a plan view of the front face of the circular slide rule of FIG. 24 showing data indicative of previous nutrition dosages and data indicative of current nutrition dosages.

FIG. 29 is a plan view of the back face of the circular slide rule of FIG. 24 showing data indicative of current glucose levels and unscaled insulin dosages.

FIG. 30—is a graphical representation of insulin sensitivities for a selected group of patients.

### DETAILED DESCRIPTION OF THE PREFERRED EMBODIMENTS

As used herein, the term “basal metabolic rate” or BMR means the minimum calorific requirement needed to sustain life in a resting individual.

As used herein, the term “resting” refers to a person having little or no mobility.

As used herein, the term “critically ill” refers to patients having higher than normal levels of insulin resistance and impaired glucose metabolism associated with at least one other illness or trauma.

As used herein, the term “diabetic” refers to patients medically recognized as having a metabolic disorder characterized by hyperglycemia (high blood glucose).

A future period of time is the next logical time period usually 1 or more hours.

The present invention now is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. The present invention will now be described more fully hereinafter with reference to the accompanying drawings, in which preferred embodiments of the invention are shown. Like numbers refer to like elements throughout.

As will be appreciated by one of skill in the art, the present invention may be embodied as a method, data processing system, or computer program product. Accordingly, the present invention may take the form of an entirely hardware embodiment, an entirely software embodiment, or an embodiment combining software and hardware aspects. Furthermore, the present invention may take the form of a computer program product on a computer-readable storage medium having computer-readable program code means embodied in the medium. Any suitable computer medium may be utilized including hard disks, CD-ROMs, optical storage devices, or magnetic storage devices. The present invention may also take the form of an analog computational device that functions as a manual calculator that utilizes physiological parameters relevant to human metabolism as inputs for determining dosing recommendations to assist a clinician in obtaining and maintaining metabolic homeostasis in critically-ill or diabetic patients.

The present invention is described below with reference to flowchart illustrations of methods, apparatus (systems), and computer program products according to embodiments of the invention. It will be understood that each block of the flowchart illustrations and combinations of blocks in the flowchart illustrations can be implemented by computer program instructions. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

These computer program instructions may also be stored in a computer-usable memory that can direct a computer or other programmable data processing apparatus to function in a particular manner, such that the instructions stored in the computer-usable memory device including instruction means which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

Accordingly, blocks of the flowchart illustrations support combinations of means for performing the specified functions, combinations of steps for performing the specified functions and program instruction means for performing the specified functions. It will also be understood that each block of the flowchart illustrations, and combinations of blocks in the flowchart illustrations, can be implemented by special purpose hardware-based computer systems which perform the specified functions or steps, or combinations of special purpose hardware and computer instructions.

Computer program for implementing the present invention may be written in various programming languages, such as Delphi, Java®, C, C++, Smalltalk, FORTRAN, COBOL, BASIC, VISUAL BASIC or any other programming language could be utilized without departing from the spirit and intent of the present invention.

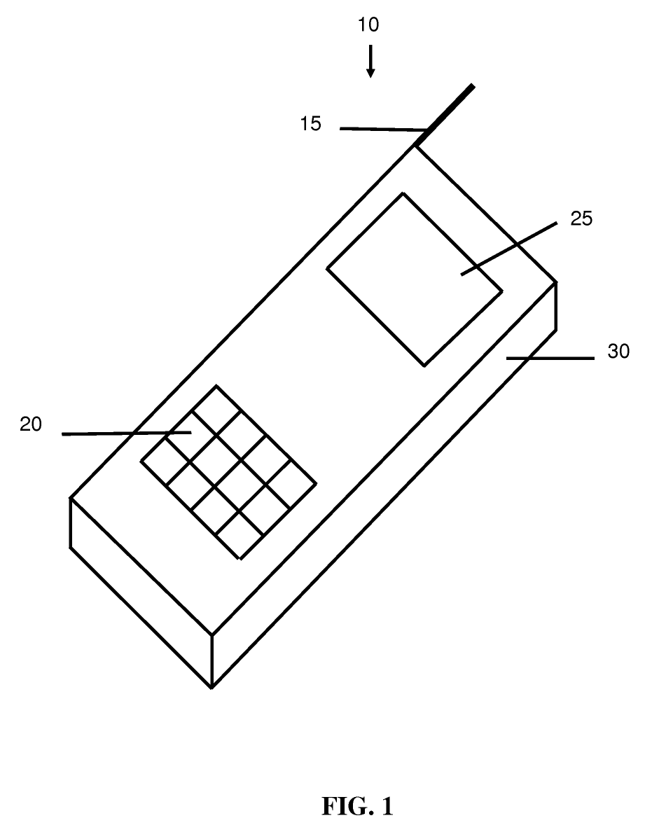

As shown in FIG. 1, one embodiment of the digital computational device of the present invention is shown generally at 10 and is hereinafter referred to as “device 10.” Device 10 includes a data-transfer module 15, an alpha-numeric input 20, a display 25, and a housing for the data processing means 30.

The use of the device 10 allows a clinician to calculate outputs relating to dosages and therapies to be administered to patients. The outputs are calculated from metabolic indicators that have specific relevance to diabetic patients, such outputs including, but not being limited to, body temperature, renal function, blood pressure, and urine output, as these metabolic indicators relate to the BMR. These outputs are of particular physiological relevance to the diabetic patients having stress-related hypertension, restricted cardiovascular systems, and other disease-related issues. In some cases, severe damage has occurred in the diabetic patients, which thereby causes them to react in manners that are different from patients in which such stresses are not present.

In one embodiment of a system of the present invention, a physiological model for use with the device 10 incorporates parameters relating to functions that include, but are not limited to, body temperature, renal function, urine output rate, blood pressure, catecholamine dosage, and the like. Each of these parameters may be used with the device 10 as described herein to calculate body status factors to titrate nutrition and insulin dosages to the current metabolic state of a critically ill or diabetic patient. This model is based on the equations shown below:

$$\begin{matrix} \dot{G} = - {p}_{G} G - {S}_{I} ( G + {G}_{E} ) \frac{Q}{1 + {α}_{G} Q} + ( \begin{matrix}{P}_{scale} \times {P}_{max} \times \\ {P}_{algorithm} ( t )\end{matrix} ) & ( 7 ) \\ \dot{Q} = - kI + kQ & ( 8 ) \\ \dot{I} = - \frac{nI}{1 + {α}_{G} Q} + \frac{( {U}_{scale} \times {U}_{max} \times U ( t ) )}{V} & ( 9 ) \\ {P}_{scale} = {k}_{TN} ( T ( t ) ) \times {k}_{KN} ( K ( t ) ) \times {k}_{DN} ( D ( t ) ) \times {k}_{BN} ( B ( t ) ) \times {k}_{NN} ( N ( t ) ) & ( 10 ) \\ {U}_{scale} = {k}_{TU} ( T ( t ) ) \times {k}_{KU} ( K ( t ) ) \times {k}_{DU} ( D ( t ) ) \times {k}_{BU} ( B ( t ) ) \times {k}_{NU} ( N ( t ) ) & ( 11 ) \\ {P}_{max} = \frac{{A}_{g} ( {W}_{e} + {H}_{e} ) {G}_{en} \times 2000}{24} & ( 12 ) \\ {U}_{max} = {( {A}_{g} ( {W}_{e} + {H}_{e} ) {G}_{en} )}^{2} & ( 13 )\end{matrix}$$

Where the inputs and outputs are:
k=Effective half-life parameter of insulin
p_{G}=Patient clearance of glucose
S_{I}=Insulin sensitivity
V=Insulin distribution volume
n=Constant 1st order decay rate of Insulin
α_{G}=Saturation parameter of insulin-stimulated glucose uptake
α_{I}=Saturation parameter of plasma insulin removal
Palgorithm(t)=Percentage nutrition rate calculated from nutrition algorithm input
Pₘₐₓ=Target nutrition rate
P_{scale}=Nutrition body status scaling factor
Uₘₐₓ=Body size insulin scaling factor
U_{scale}=Body status insulin scaling factor
U(t)=Exogenous insulin
T(t)=Body temperature input
K(t)=Renal function input
D(t)=Hourly urine input
B(t)=Blood pressure input
N(t)=Catecholamine dosage input
K_{TN}=Temperature nutrition factor
K_{KN}=Renal nutrition factor
K_{DN}=Urine nutrition factor
K_{BN}=Blood pressure nutrition factor
K_{NN}=Catecholamine nutrition factor
K_{TU}=Temperature insulin factor
K_{KU}=Renal insulin factor
K_{DU}=Urine insulin factor
K_{BU}=Blood pressure insulin factor
K_{NU}=Catecholamine insulin factor
A_{g}=Age factor
Wₑ=Weight factor
Hₑ=Height factor
Gₑₙ=Gender factor

The model presented by the above equations represents a closed-loop differential equation model that operates as an algorithm capable of generating knowledge-based therapy recommendations for a patient. The model also provides a method for preventing and/or delaying the onset of diabetes in a patient as well as managing and/or treating diabetes or maintaining the glucose and insulin levels in a critically ill patient. Equations 7-9 are derived from an initial model of glucose and insulin kinetics developed from work that is based upon physiological insulin models described in the Examples herein. Equations 10-13 provide factors for calculating the relevant recommended nutrition dosages.

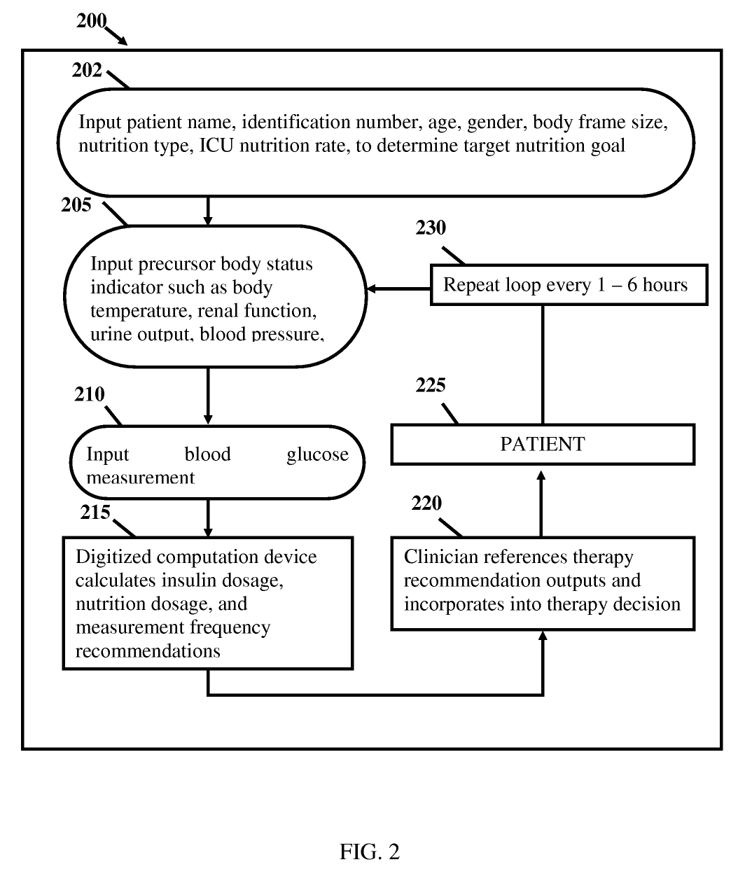

Referring now to FIG. 2, one exemplary method of using the device to determine a recommended nutrition and insulin dosage for a critically ill and/or diabetic patient is shown generally at 200 and is hereinafter referred to as “recommendation method 200.” In the recommendation method 200, the user must first input patient information required to set-up the program 202. This information includes but is not limited to patient name, identification number, age, gender, body frame size, ICU nutrition rate, and nutrition type.

After set-up and initiation of the program recommendation method 200 is used in an iterative methodology to optimize care given to the patient. In step 205 the clinician inputs the current value of the precursor body status indicator. Precursor indicators include but are not limited to body temperature, renal function, urine output, blood pressure, and catecholamine dosage. In step 210 the clinician inputs the current blood glucose measurement. Step 210 is compatible with any and all FDA-approved blood glucose measuring techniques including but not limited to continuous blood glucose monitoring systems and single use test strip meters.

In step 215 of recommendation method 200 the digital computation device utilizes the aforementioned inputs from step 202, 205, and 210 to determine an insulin dosage, nutrition dosage, and measurement frequency recommendation.

In an incorporation step 220, the clinician then references the recommended absolute insulin and nutrition rate from the calculation step 215 and incorporates this value into a therapy decision. Additional changes to the nutrition rate can be made as required including but not limited to increase or decrease for minimum or maximum fluid required, increase or decrease to compensate for needed amino acids, increase or decrease for minimum or maximum protein required. Additional changes can be made subject to occurrence of diarrhea, abdominal distention, nausea or vomiting or due to high gastric residual volume or any observed electrolyte abnormalities.

In a delivery step 225, the absolute nutrition and insulin rates based on the therapy decision made pursuant to the incorporation step 220 is delivered to the patient. A repeat step 230 provides for control of a loop over selected time intervals (for example, every 1-6 hours and incorporating the blood glucose measurement frequency recommendation calculated in step 215), thereby ensuring a consistent therapy and level of care. The recommended blood glucose measurement frequency determined in step 215 is incorporated in step 230.

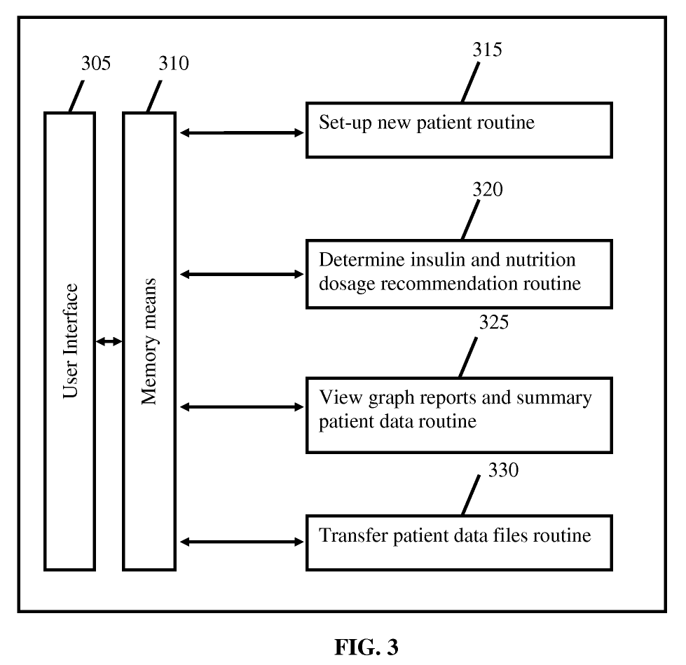

FIG. 3 displays the structure of the software program. Block 305 is the user interface enabling both the receipt of data from the clinician user and a means for displaying output recommendations. Block 310 is the memory means consisting of any computer-readable storage medium having computer-readable program code means embodied in the medium. Any suitable computer medium may be utilized including hard disks, CD-ROMs, optical storage devices, or magnetic storage devices.

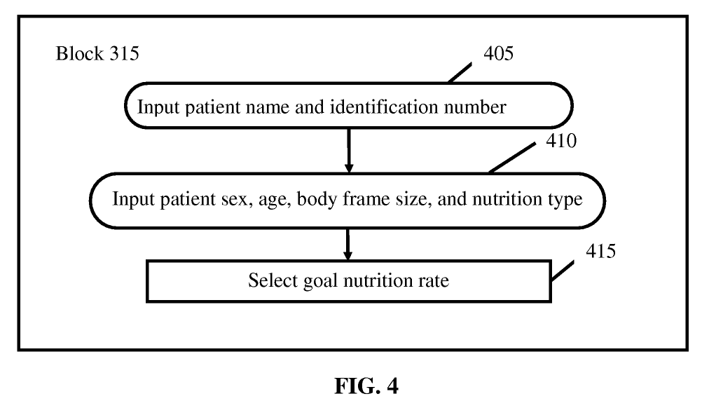

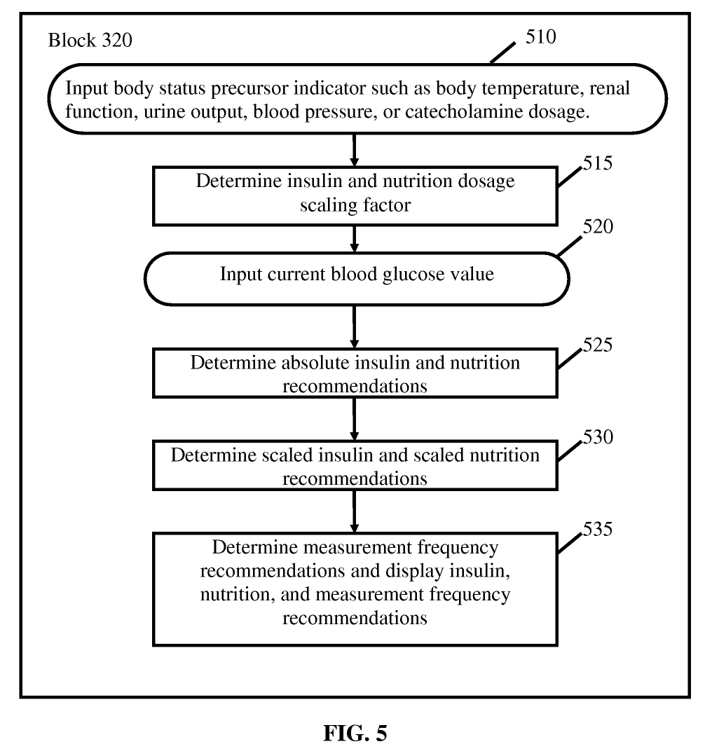

The software consists of four basic routines that are executed as required by the user. Routine 315 is required to set-up a new patient and the operations contained in block 315 are illustrated schematically in FIG. 4. Routine 320 is performed during each control loop to determine insulin, nutrition, and measurement frequency recommendations, and the operations contained in block 320 are illustrated schematically in FIG. 5.

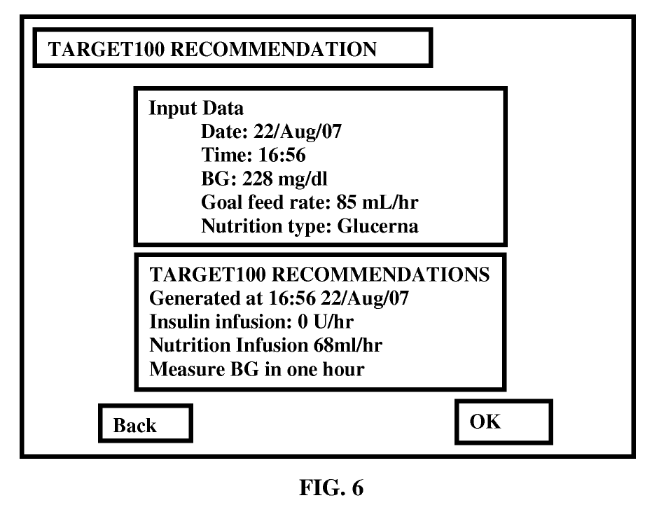

Routine 325 is executed if the user would like to view a report on patient data. FIG. 6 is the report for the clinical provider showing recommended insulin and nutrition.

Routine 330 is executed if the user would like to transfer patient data from the device to another location. Electronic data transfer includes but is not limited an Internet connection, Bluetooth, WIFI or other radio communication protocol, USB, firewire, serial or parallel ports, compact discs, flash memory devices or any other wired or wireless data transfer technology. An Internet connection may be made via a modem connected to traditional phone lines, an ISDN link, a T1 link, a T3 link, via cable television, via an ethernet network, and the like. An Internet connection may be made via a third party, such as an “Internet Service Provider” (“ISP”). Alternative manual-based data transfer methods such as but not limited to printed material may also be used.

FIG. 4 displays the operations required to perform the set-up new patient routine. After initializing the new patient routine the user must first enter the patient name and identification number in input step 405. In input step 410 the user will select the patient sex, age, body frame size, ICU nutrition rate, and nutrition type from specified ranges.

The patient sex is chosen from the choices listed in Table 1 below.

TABLE 1

| | |
|---|---|
| Male (S1) | Female (S2) |

The patient age is chosen from the choices listed in Table 2. This list is not meant to be exhaustive alternative groups are possible.

TABLE 2

| | | | |
|---|---|---|---|
| 15-39 yrs (A1) | 40-59 yrs (A2) | 60-79 yrs (A3) | 80+ yrs (A4) |

The patient body frame is chosen from the choices listed in Table 3. This list is not meant to be exhaustive alternative groups are possible.

TABLE 3

| | | |
|---|---|---|
| Small (<130 lbs) (B1) | Medium (130-175 lbs) (B2) | Big (>175 lbs) (B3) |

The nutrition formulation is chosen from the enteral nutrition formulation choices listed in Table 4. This list is not meant to be exhaustive, alternative enteral or parenteral nutrition formulations such as but not limited to, Isosource VHN, Nutren 1.5, Peptinex DT with Prebio, Subdue Plus, Gyltrol with Prebio, Nutren Renal, Suplena, Fibresource HN, Isosource 1.5 Cal, Isosource, Isosource HN, Ultracal, Isocal HN, and Nutren are possible. Additionally, a parenteral nutrition formulation with a dextrose content between 0-35% and an amino acid content between 0-6% may also be used.

TABLE 4

| | |
|---|---|
| Diabetic Resource | Glucerna |
| (N1) | (N2) |

The ICU nutrition rate is chosen from the choices listed in Table 5. This list is not mean to be exhaustive alternative groups are possible.

TABLE 5

| | | |
|---|---|---|
| <25 Calories/kg/day | 25-30 Calories/kg/day | >30 Calories/kg/day |
| (Y1) | (Y2) | (Y3) |

The inputs in step 410 are used in a look-up routine performed in block 415 to determine the goal nutrition rate for the patient. The look-up table is presented in Table 6.

TABLE 6

| Patient sex, age, | Nutrition |
|---|---|
| body frame, and | Goal |
| nutrition | (ml/hr) |
| S1 A1 B1 N1 | 80 |
| Y1 | |
| S1 A2 B1 N1 | 75 |
| Y1 | |
| S1 A3 B1 N1 | 70 |
| Y1 | |
| S1 A4 B1 N1 | 60 |
| Y1 | |
| S1 A1 B2 N1 | 90 |
| Y1 | |
| S1 A2 B2 N1 | 85 |
| Y1 | |
| S1 A3 B2 N1 | 75 |
| Y1 | |
| S1 A4 B2 N1 | 65 |
| Y1 | |
| S1 A1 B3 N1 | 100 |
| Y1 | |
| S1 A2 B3 N1 | 90 |
| Y1 | |
| S1 A3 B3 N1 | 80 |
| Y1 | |
| S1 A4 B3 N1 | 75 |
| Y1 | |
| S2 A1 B1 N1 | 65 |
| Y1 | |
| S2 A2 B1 N1 | 60 |
| Y1 | |
| S2 A3 B1 N1 | 55 |
| Y1 | |
| S2 A4 B1 N1 | 50 |
| Y1 | |
| S2 A1 B2 N1 | 75 |
| Y1 | |
| S2 A2 B2 N1 | 65 |
| Y1 | |
| S2 A3 B2 N1 | 60 |
| Y1 | |
| S2 A4 B2 N1 | 55 |
| Y1 | |
| S2 A1 B3 N1 | 80 |
| Y1 | |
| S2 A2 B3 N1 | 75 |
| Y1 | |
| S2 A3 B3 N1 | 65 |
| Y1 | |
| S2 A4 B3 N1 | 60 |
| Y1 | |
| S1 A1 B1 N2 | 80 |
| Y1 | |
| S1 A2 B1 N2 | 75 |
| Y1 | |
| S1 A3 B1 N2 | 70 |
| Y1 | |
| S1 A4 B1 N2 | 60 |
| Y1 | |
| S1 A1 B2 N2 | 90 |
| Y1 | |
| S1 A2 B2 N2 | 85 |
| Y1 | |
| S1 A3 B2 N2 | 75 |
| Y1 | |
| S1 A4 B2 N2 | 65 |
| Y1 | |
| S1 A1 B3 N2 | 100 |
| Y1 | |
| S1 A2 B3 N2 | 90 |
| Y1 | |
| S1 A3 B3 N2 | 80 |
| Y1 | |
| S1 A4 B3 N2 | 75 |
| Y1 | |
| S2 A1 B1 N2 | 65 |
| Y1 | |
| S2 A2 B1 N2 | 60 |
| Y1 | |
| S2 A3 B1 N2 | 55 |
| Y1 | |
| S2 A4 B1 N2 | 50 |
| Y1 | |
| S2 A1 B2 N2 | 75 |
| Y1 | |
| S2 A2 B2 N2 | 65 |
| Y1 | |
| S2 A3 B2 N2 | 60 |
| Y1 | |
| S2 A4 B2 N2 | 55 |
| Y1 | |
| S2 A1 B3 N2 | 80 |
| Y1 | |
| S2 A2 B3 N2 | 75 |
| Y1 | |
| S2 A3 B3 N2 | 65 |
| Y1 | |
| S2 A4 B3 N2 | 60 |
| Y1 | |
| S1 A1 B1 N1 | 85 |
| Y2 | |
| S1 A2 B1 N1 | 80 |
| Y2 | |
| S1 A3 B1 N1 | 75 |
| Y2 | |
| S1 A4 B1 N1 | 65 |
| Y2 | |
| S1 A1 B2 N1 | 95 |
| Y2 | |
| S1 A2 B2 N1 | 90 |
| Y2 | |
| S1 A3 B2 N1 | 80 |
| Y2 | |
| S1 A4 B2 N1 | 70 |
| Y2 | |
| S1 A1 B3 N1 | 105 |
| Y2 | |
| S1 A2 B3 N1 | 95 |
| Y2 | |
| S1 A3 B3 N1 | 85 |
| Y2 | |
| S1 A4 B3 N1 | 80 |
| Y2 | |
| S2 A1 B1 N1 | 70 |
| Y2 | |
| S2 A2 B1 N1 | 65 |
| Y2 | |
| S2 A3 B1 N1 | 60 |
| Y2 | |
| S2 A4 B1 N1 | 55 |
| Y2 | |
| S2 A1 B2 N1 | 80 |
| Y2 | |
| S2 A2 B2 N1 | 70 |
| Y2 | |
| S2 A3 B2 N1 | 65 |
| Y2 | |
| S2 A4 B2 N1 | 60 |
| Y2 | |
| S2 A1 B3 N1 | 85 |
| Y2 | |
| S2 A2 B3 N1 | 80 |
| Y2 | |
| S2 A3 B3 N1 | 70 |
| Y2 | |
| S2 A4 B3 N1 | 65 |
| Y2 | |
| S1 A1 B1 N2 | 85 |
| Y2 | |
| S1 A2 B1 N2 | 80 |
| Y2 | |
| S1 A3 B1 N2 | 75 |
| Y2 | |
| S1 A4 B1 N2 | 65 |
| Y2 | |
| S1 A1 B2 N2 | 95 |
| Y2 | |
| S1 A2 B2 N2 | 90 |
| Y2 | |
| S1 A3 B2 N2 | 80 |
| Y2 | |
| S1 A4 B2 N2 | 70 |
| Y2 | |
| S1 A1 B3 N2 | 105 |
| Y2 | |
| S1 A2 B3 N2 | 95 |
| Y2 | |
| S1 A3 B3 N2 | 85 |
| Y2 | |
| S1 A4 B3 N2 | 80 |
| Y2 | |
| S2 A1 B1 N2 | 70 |
| Y2 | |
| S2 A2 B1 N2 | 65 |
| Y2 | |
| S2 A3 B1 N2 | 60 |
| Y2 | |
| S2 A4 B1 N2 | 55 |
| Y2 | |
| S2 A1 B2 N2 | 80 |
| Y2 | |
| S2 A2 B2 N2 | 70 |
| Y2 | |
| S2 A3 B2 N2 | 65 |
| Y2 | |
| S2 A4 B2 N2 | 60 |
| Y2 | |
| S2 A1 B3 N2 | 85 |
| Y2 | |
| S2 A2 B3 N2 | 80 |
| Y2 | |
| S2 A3 B3 N2 | 70 |
| Y2 | |
| S2 A4 B3 N2 | 65 |
| Y2 | |
| S1 A1 B1 N1 | 90 |
| Y3 | |
| S1 A2 B1 N1 | 85 |
| Y3 | |
| S1 A3 B1 N1 | 80 |
| Y3 | |
| S1 A4 B1 N1 | 70 |
| Y3 | |
| S1 A1 B2 N1 | 100 |
| Y3 | |
| S1 A2 B2 N1 | 95 |
| Y3 | |
| S1 A3 B2 N1 | 85 |
| Y3 | |
| S1 A4 B2 N1 | 75 |
| Y3 | |
| S1 A1 B3 N1 | 110 |
| Y3 | |
| S1 A2 B3 N1 | 100 |
| Y3 | |
| S1 A3 B3 N1 | 90 |
| Y3 | |
| S1 A4 B3 N1 | 85 |
| Y3 | |
| S2 A1 B1 N1 | 75 |
| Y3 | |
| S2 A2 B1 N1 | 70 |
| Y3 | |
| S2 A3 B1 N1 | 65 |
| Y3 | |
| S2 A4 B1 N1 | 60 |
| Y3 | |
| S2 A1 B2 N1 | 85 |
| Y3 | |
| S2 A2 B2 N1 | 75 |
| Y3 | |
| S2 A3 B2 N1 | 70 |
| Y3 | |
| S2 A4 B2 N1 | 65 |
| Y3 | |
| S2 A1 B3 N1 | 90 |
| Y3 | |
| S2 A2 B3 N1 | 85 |
| Y3 | |
| S2 A3 B3 N1 | 75 |
| Y3 | |
| S2 A4 B3 N1 | 70 |
| Y3 | |
| S1 A1 B1 N2 | 90 |
| Y3 | |
| S1 A2 B1 N2 | 85 |
| Y3 | |
| S1 A3 B1 N2 | 80 |
| Y3 | |
| S1 A4 B1 N2 | 70 |
| Y3 | |
| S1 A1 B2 N2 | 100 |
| Y3 | |
| S1 A2 B2 N2 | 95 |
| Y3 | |
| S1 A3 B2 N2 | 85 |
| Y3 | |
| S1 A4 B2 N2 | 75 |
| Y3 | |
| S1 A1 B3 N2 | 110 |
| Y3 | |
| S1 A2 B3 N2 | 100 |
| Y3 | |
| S1 A3 B3 N2 | 90 |
| Y3 | |
| S1 A4 B3 N2 | 85 |
| Y3 | |
| S2 A1 B1 N2 | 75 |
| Y3 | |
| S2 A2 B1 N2 | 70 |
| Y3 | |
| S2 A3 B1 N2 | 65 |
| Y3 | |
| S2 A4 B1 N2 | 60 |
| Y3 | |
| S2 A1 B2 N2 | 85 |
| Y3 | |
| S2 A2 B2 N2 | 75 |
| Y3 | |
| S2 A3 B2 N2 | 70 |
| Y3 | |
| S2 A4 B2 N2 | 65 |
| Y3 | |
| S2 A1 B3 N2 | 90 |
| Y3 | |
| S2 A2 B3 N2 | 85 |
| Y3 | |
| S2 A3 B3 N2 | 75 |
| Y3 | |
| S2 A4 B3 N2 | 70 |
| Y3 | |

The routine contained in block 320 is performed iteratively every 1-6 hours, or every recommended measurement frequency, to determine a new insulin dosage, a new nutritional dosage, and blood glucose measurement frequency. The operational steps performed in block 320 are illustrated in FIG. 5. In input step 510 the clinician inputs the appropriate body status precursor indicator based on the current patient condition. The body status indicators may be entered in as numeric values, via drop-down boxes, or selected from a selection of specified ranges. If body temperature is used the selection may be made from the choices presented in Table 7. This list is not meant to be exhaustive, alternative groupings are possible.

TABLE 7

| | | |
|---|---|---|
| Body temperature | Body temperature is less than | Body temperature |
| is less than 36° C. | 39° C. and greater than 36° C. | is greater than 39° C. |
| (T1) | (T2) | (T3) |

In computing step 515 the selected body status precursor indicator is used to determine an insulin and nutrition dosage scaling factor. Scaling factors for body temperature are stored below in Table 8. A simple look-up operation procedure is performed.

TABLE 8

| | Insulin scaling | Nutrition scaling |
|---|---|---|
| Precursor Indicator | factor | factor |
| Body temperature is less than | 1.14 | 0.88 |
| 36° C. (T1) | | |
| Body temperature is less than | 1.00 | 1.00 |
| 39° C. and greater than 36° C. (T2) | | |
| Body temperature is greater | 1.14 | 1.07 |
| than 39° C. (T3) | | |

After the appropriate insulin and nutrition scaling factors have been selected the clinician is prompted to input the current blood glucose value of the patient in input step 520. This input may be a numeric input, via drop down boxes, or alternatively from a grouping of specified blood glucose ranges.

In step 525 the absolute insulin and absolute nutrition recommendations are determined. To determine the absolute insulin recommendation the operation calls the previous absolute insulin infusion rate and current blood glucose value (from step 520) from memory. These two inputs are used in a look-up table to determine a new absolute insulin recommendation. A sample look-up table is shown below in Table 9.

TABLE 9

| | | Previous insulin infusion (U/Hr) | | | | | | |
|---|---|---|---|---|---|---|---|---|
| | | 0-1 | 1-2 | 2-3 | 3-4 | 4-5 | 5-6 | 6-7 |
| Blood glucose (mmol/L) | <4.4 | 0 | 0 | 0 | 0 | 0 | 0 | 0 |
| | 4.4-5 | 1 | 2 | 2 | 2 | 2 | 2 | 2 |
| | 5-6.1 | 1 | 2 | 3 | 3 | 3 | 3 | 3 |
| | 6.1-7.2 | 3 | 3 | 4 | 4 | 5 | 6 | 6 |
| | 7.2-8.8 | 3 | 4 | 4 | 4 | 5 | 5 | 5 |
| | 8.8-10.5 | 4 | 4 | 4 | 5 | 5 | 5 | 5 |
| | 10.5-12.7 | 4 | 5 | 5 | 5 | 5 | 5 | 5 |
| | 12.7-13.8 | 5 | 5 | 5 | 5 | 5 | 6 | 5 |
| | >13.8 | 5 | 5 | 5 | 6 | 6 | 6 | 5 |

To determine a new absolute nutrition recommendation the operation 525 calls the previous absolute nutrition rate and current blood glucose value (from 520) from memory. These two inputs are used in a look-up table to determine a new absolute nutrition rate. A sample look-up table is shown below in Table 10.

TABLE 10

| | | Previous nutrition rate (ml/hr) | | | | | | | |
|---|---|---|---|---|---|---|---|---|---|
| | | 30 | 40 | 50 | 60 | 70 | 80 | 90 | 100 |
| Blood | <4.4 | 40 | 50 | 60 | 70 | 80 | 90 | 100 | 100 |
| glucose | 4.4-6.1 | 30 | 40 | 50 | 60 | 70 | 80 | 90 | 100 |
| (mmol/L) | >6.1 | 30 | 30 | 40 | 50 | 60 | 70 | 80 | 90 |

In operation 530 the scaled insulin and nutrition dosage recommendations are determined by multiplying the absolute nutrition rate and the absolute insulin rate from operation 525 by the insulin and nutrition scaling factors from computing step 515.

To determine the new measurement frequency operation 535 calls the current blood glucose value (from 520) from memory. This input is used in a look-up table to determine the new measurement frequency. A sample look-up table is shown in Table 11.

TABLE 11

| Previous blood glucose (mmol/L) | New measurement frequency (hours) |
|---|---|
| <6.1 | 1 |
| >6.1 | 2 |

In step 535 the insulin, nutrition, and measurement frequency recommendations are displayed to the clinician via the user-interface as illustrated in FIG. 6.

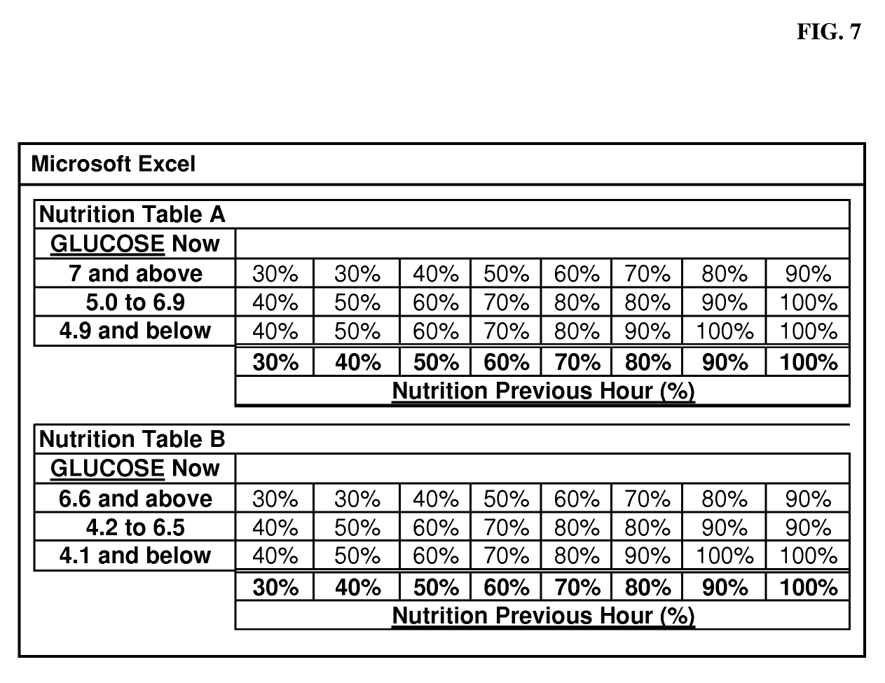

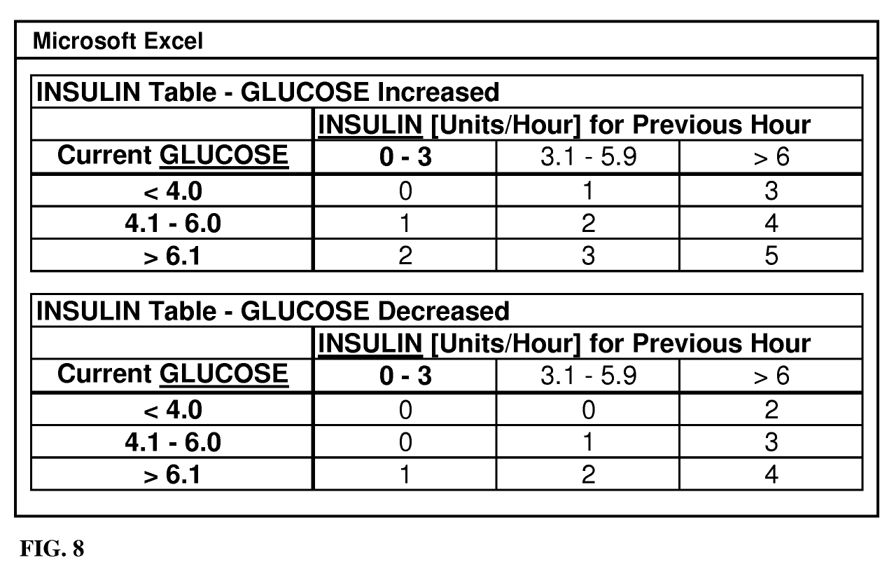

For example, FIG. 7 illustrates nutrition tables for use with computer graphic embodiments of the present invention. Also, FIG. 8 illustrates insulin tables for use with computer graphic embodiments of the present invention.

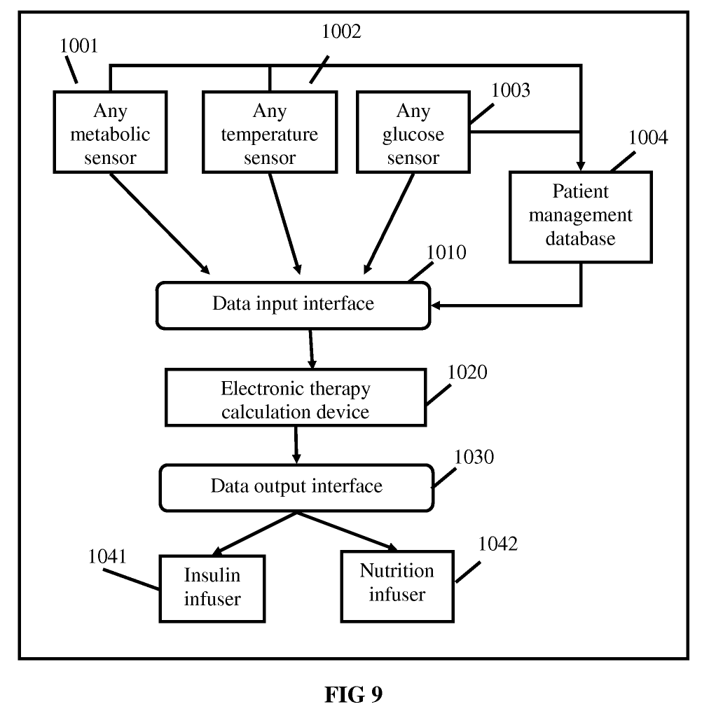

FIG. 9 describes the possible data input and output interfaces to the electronic device. These interfaces enable an integration of the device into commonly used patient management systems available in an ICU to reduce the workload on medical personnel. Block 1001-1003 show the metabolic sensors used to obtain the relevant metrics required by the algorithm. This data can be transferred to the data input interface 1010 directly or indirectly via a patient data management system 1004 already in place in the ICU. The input interface 1010 presents the data to the electronic therapy calculation device 1020, an embodiment of the presented invention. The determined therapeutic interventions are transferred via the data output interface 1030 to the insulin and nutrition administration infusers, 1041-1042. The therapeutic decision can be performed automatically by the infusers in a closed loop system, or require an additional acknowledgement by clinical personnel. Confirmatory signals may be received from the data output interface 1030 to a display for visual confirmation by appropriate personnel.

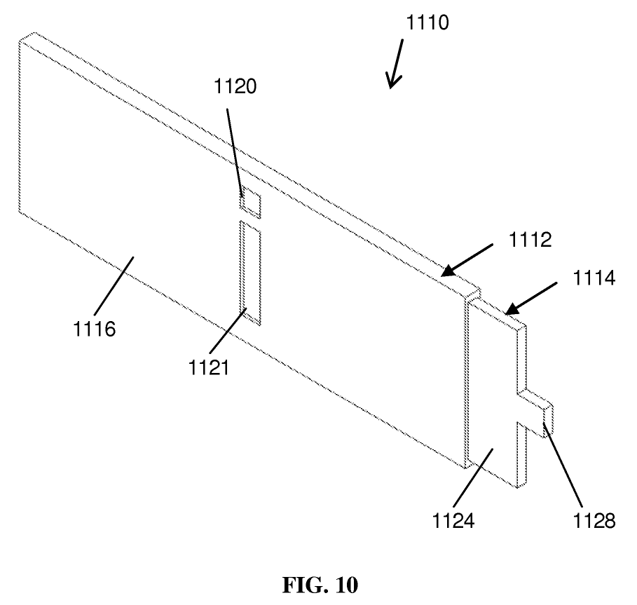

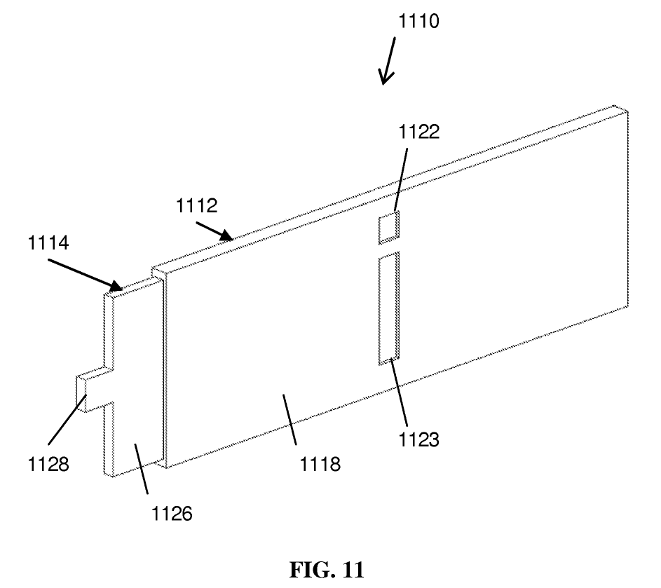

As shown in FIGS. 10 and 11, one embodiment of the analog computational device of the present invention is shown generally at 1110 and is hereinafter referred to as “device 1110.” Device 1110 includes a pocket 1112 and a slide member 1114 slidably positioned therein. The pocket 1112 is defined by a front panel 1116 and a back panel 1118 and is arranged to define at least one open end through which the slide member 1114 may slide.

The front panel 1116 and the back panel 1118 may be connected along two opposing edges thereof using any suitable means. In the alternative, at least one of the edges of the pocket 1112 may be defined by a fold formed in a sheet of material of which the pocket is fabricated. Materials from which the front panel 1116 and the back panel 1118 can be fabricated include, but are not limited to, paper (for example cardstock), plastic, combinations of the foregoing materials, and the like.

As can be seen in FIG. 10, the front panel 1116 includes an upper front window 1120 and a lower front window 1121, and as can be seen in FIG. 11, the back panel 1118 includes an upper back window 1122 and a lower back window 1123. The upper front window 1120, the lower front window 1121, the upper back window 1122, and the lower back window 1123 each enable data printed on the slide member 1114 to be viewed through the respective windows.

The slide member 1114 is a planar member that can be moved in the pocket 1112 between the front panel 1116 and the back panel 1118. The slide member 1114 includes a front face 1124 and a back face 1126 for cooperation with the front panel 1116 through the upper front window 1120 and the lower front window 1121 and for cooperation with the back panel 1118 through the upper back window 1122 and the lower back window 1123. The dimensions of the slide member 1114 are such that it can be held in a desired position in the pocket 1112 via frictional engagement with the pocket. Materials from which the slide member can be fabricated include, but are not limited to, paper (for example cardstock), plastic, combinations of the foregoing materials, and the like. A tab 1128 may be attached to the slide member 1114 to facilitate the movement of the slide member in the pocket 1112. The present invention is not limited to a tab, however, as rings, knobs, textured surfaces, and the like may be used.

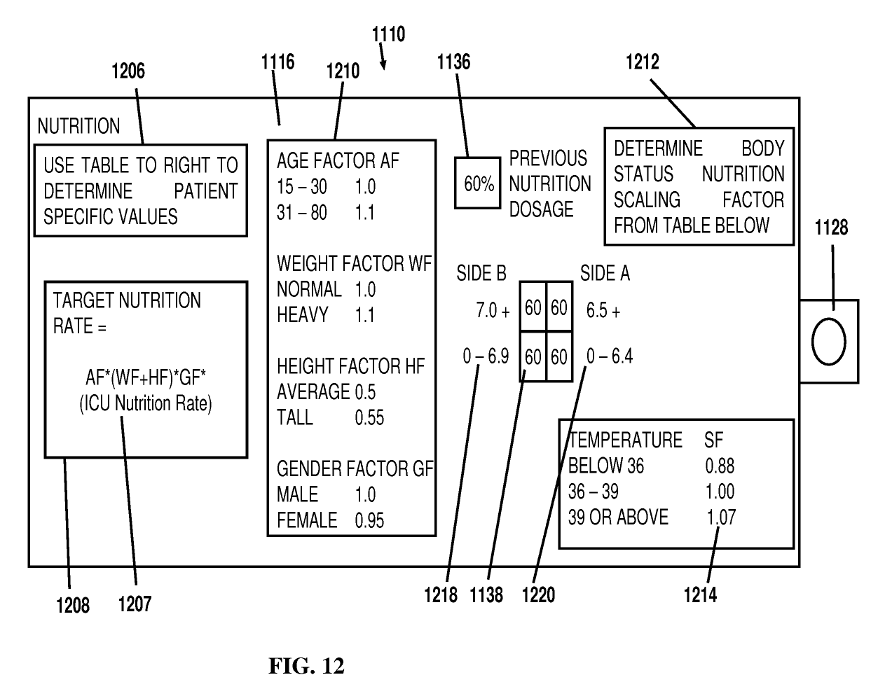

Referring now to FIG. 12, instructional text and calculating information is printed on the front panel 1116 of the device 1110. In one embodiment, such text and information includes instructions 1206 that provide for calculating an initial scaling factor for a patient from body size, gender, and age parameters provided in a table 1210 and multiplying this value by a user-defined standard ICU rate, entered in as variable 1207, to determine a target nutrition rate in equation 1208. The ICU nutrition rate may differ from intensive care unit to intensive care unit and is defined as the nutrition rate that the ICU usually gives its critically ill patients. These body size and age parameters are patient specific physical attributes. The target nutrition rate may be calculated from these parameters in any suitable volume as a function of time, such as milliliters per hour (ml/hr).

Instructions 1212 also provide for determining a body status nutrition scaling factor from the current body temperature of the patient using a table 1214. The table 1214 provides a range of body temperature values and associated scaling factors. The clinician references the table 1214 to select the appropriate scaling factor based on the current body temperature of the patient.

The present invention is not limited to the printing of table 1210 or table 1214 onto the front panel 1116, however, as the information provided therein may be provided elsewhere.

A previous nutrition dosage value 1136 is viewable through the upper front window, and a current nutrition dosage value 1138 is viewable through the lower front window. A first current blood glucose value table 1218 and a second current blood glucose value table 1220 are located on opposing lateral sides of the lower front window in the front panel 1116. The first current blood glucose value table 1218 is indicative of the blood glucose level of the patient when the previous blood glucose level has not decreased by more than a preselected amount over a given time period. The second current blood glucose value table 1220 is indicative of the blood glucose level of the patient when the previous blood glucose level has decreased by more than a preselected amount over the same given time period. In one embodiment of the present invention, the first current blood glucose value table 1218 is used when the previous blood glucose level has not decreased by more than 1.5 mmol/L over the past hour, and the second current blood glucose value table 1220 is used when the previous blood glucose level has decreased by more than 1.5 mmol/L over the past hour.

In one embodiment of the present invention, the first current blood glucose value table 1218 and the second current blood glucose value table 1220 are each arranged to define four ranges of blood glucose values. If the patient has blood glucose measurements below 4 mmol/L, use of the device 1110 will allow a clinician to calculate an increase in the current percentage nutrition rate. In addition, even if the patient is in the target 4-6 mmol/L range, an increase in feed in an effort to bring the nutrition level up to 100% of the target nutrition rate can be calculated. Depending upon the previous and current nutrition dosages, however, only a percent of the target nutrition rate may be calculated and recommended to be administered to the patient for a future period of time. In this case, the future period of time is one hour. However, the present invention is not limited in this regard, and the period of time may be longer or shorter depending on basic scale calculations and the tightness of control required. Typically, a lower limit of about 30% of the target nutrition rate is imposed to ensure that the nutrition rates administered are within safe, acceptable limits.

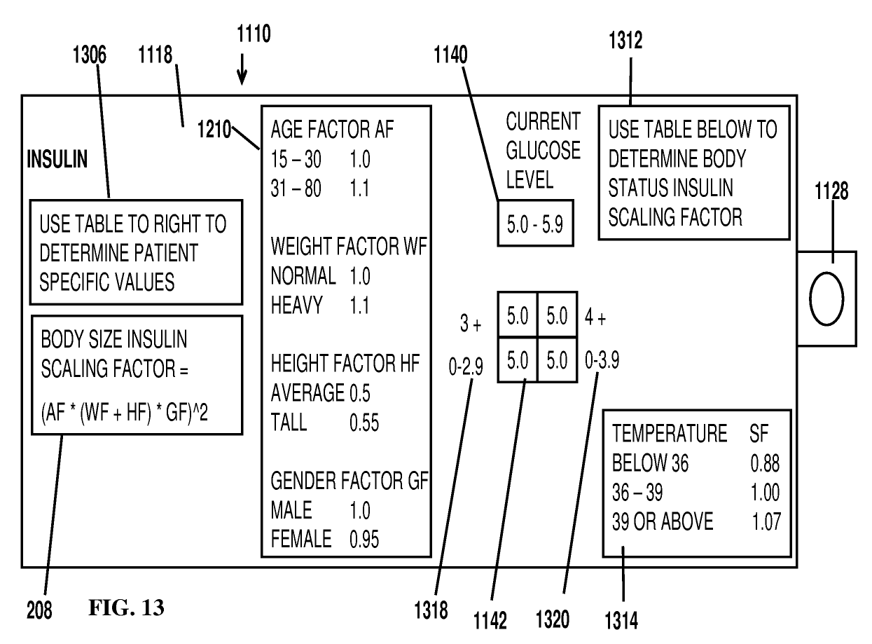

As shown in FIG. 13, instructional text and calculating information is also printed on the back panel 1118 of the device 1110. In one embodiment, this text and information includes instructions 1306 that provide for calculating a body size insulin scaling factor from a body size insulin scaling factor equation 208 for the patient from body size and parameters provided in the table 1210.

Instructions 1312 also provide for determining a body status insulin scaling factor from the current body temperature of the patient using a table 1314. The table 1314 provides a range of body temperature values and associated scaling factors.

The present invention is not limited to the printing of table 1210 or table 1314 onto the back panel 1118, however, as the information provided therein may be provided elsewhere.

A current blood glucose level 1140 is viewable through the upper back window, and an unscaled insulin dosage 1142 is viewable through the lower back window. A first insulin dosage value table 1318 and a second insulin dosage value table 1320 are located on opposing lateral sides of the lower back window in the back panel 1118. The first insulin dosage value table 1318 is indicative of the insulin dosage administered to the patient when the blood glucose level has increased by more than a preselected amount over a given time period. The second insulin dosage value table 1320 is indicative of the insulin dosage administered to the patient when the blood glucose level has decreased by more than a preselected amount over the same given time period (e.g., one hour, but which may be longer or shorter depending on the basic scale calculations).

In one embodiment of the present invention, the first insulin dosage value table 1318 and the second insulin dosage value table 1320 are each arranged to define seven insulin dosage values, namely, zero through 6 units per hour (U/hr) in 1 U/hr increments. The insulin dosage values are capped at 6 U/hr because any insulin beyond this amount is ineffective due to saturation of the insulin effect.

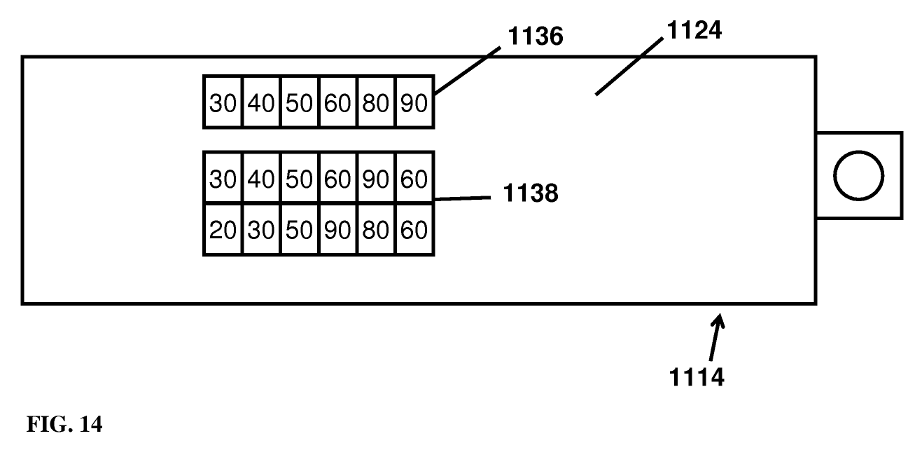

As shown in FIG. 14, the front face 1124 of the slide member 1114 includes data indicative of the previous nutrition dosages 1136 as well as data indicative of current nutrition dosages 1138. The previous percentages of nutrition dosages 1136 and the current nutrition dosages 1138 are arranged into an array such that particular previous percentages correspond to particular current dosages.

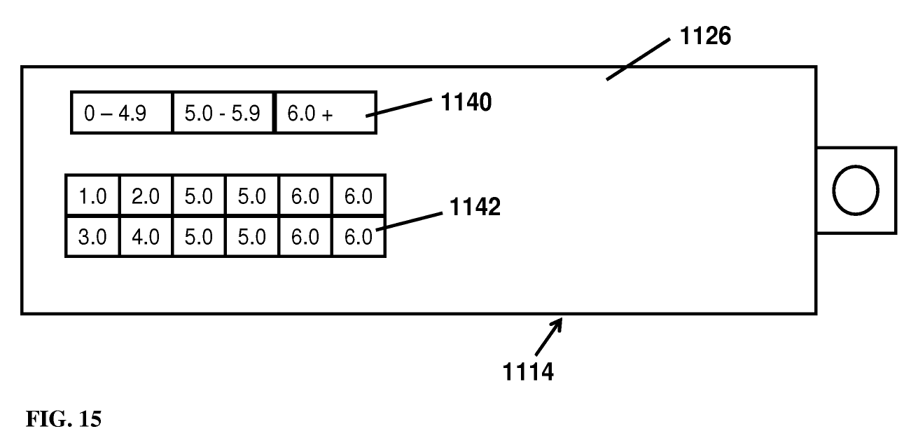

As shown in FIG. 15, the back face 1126 of the slide member 1114 includes data indicative of the current glucose level 1140 as a range and the unscaled insulin dosages 1142. The values for the current glucose levels 1140 and the unscaled insulin dosages 1142 are arranged into an array such that particular current glucose levels correspond to particular unscaled insulin dosages.

The use of the device 1110 allows a clinician to calculate outputs relating to dosages and therapies to be administered to patients. The outputs are calculated from metabolic indicators that have specific relevance to diabetic patients, such outputs including, but not being limited to, body temperature, renal function, blood pressure, and urine output, as these metabolic indicators relate to the BMR. These outputs are of particular physiological relevance to the diabetic patients having stress-related hypertension, restricted cardiovascular systems, and other disease-related issues. In some cases, severe damage has occurred in the diabetic patients, which thereby causes them to react in manners that are different from patients in which such stresses are not present.

In one embodiment of a system of the present invention, a physiological model for use with the device 1110 incorporates parameters relating to functions that include, but are not limited to, body temperature, renal function, urine output rate, blood pressure, catecholamine dosage, and the like. Each of these parameters may be used with the device 1110 as described herein to calculate body status factors to titrate nutrition and insulin dosages to the current metabolic state of a critically ill or diabetic patient. This model is also based on the equations 7-13 described above.

The model presented by the above equations represents a closed-loop differential equation model that operates as an algorithm capable of generating knowledge-based therapy recommendations for a patient. The model also provides a method for preventing and/or delaying the onset of diabetes in a patient as well as managing and/or treating diabetes or maintaining the glucose and insulin levels in a critically ill patient. Equations 7-9 (described above) are derived from an initial model of glucose and insulin kinetics developed from work that is based upon physiological insulin models described in the Examples herein. Equations 10-13 (described above) provide factors for calculating the relevant recommended nutrition dosages.

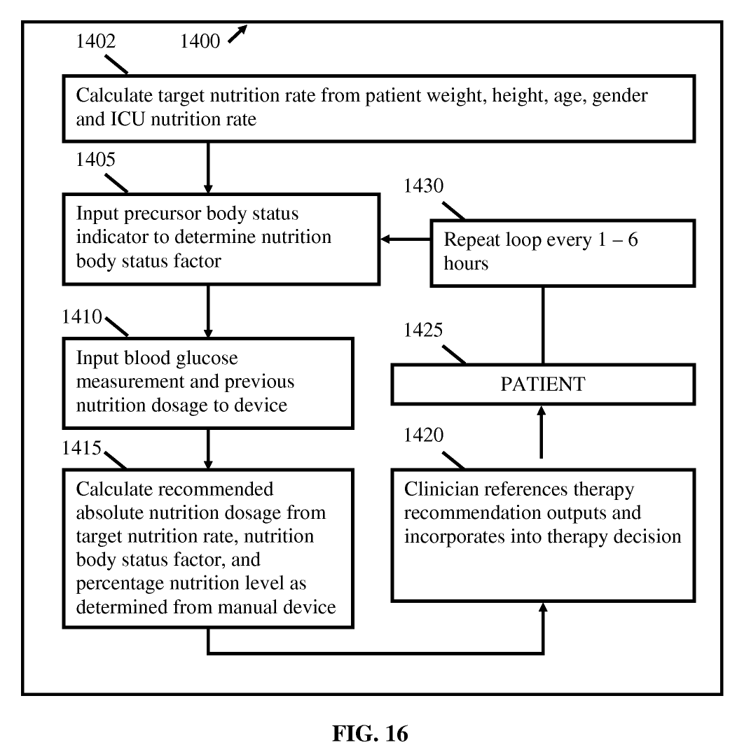

Referring now to FIG. 16, one exemplary method of using the device to determine a recommended nutrition dosage for a critically ill and/or diabetic patient is shown generally at 1400 and is hereinafter referred to as “nutrition dosage method 1400.” In the nutrition dosage method 1400, the target nutrition rate 1208 (FIG. 12) is calculated in a target nutrition rate calculation step 1402 pursuant to the instructions in the table 1206 (FIG. 12) and from the appropriate values corresponding to the patient from table 1210 (FIG. 12). The appropriate values corresponding to patient weight, height, gender, and age from table 1210 are entered into equation 1208 along with the ICU nutrition rate entered as 1207 (FIG. 12), and the target nutrition rate for the patient is determined.

In an indicator input step 1405, precursor status indicators are incorporated to assess the current status of the patient. These indicators are real-time parameters that are indicative of the patient's metabolism. Although body temperature is used as the precursor status indicator in the indicator input step 1405, the present invention is not limited in this regard, and other precursor status indicators may be used. Other precursor status indicators that may be input include, but are not limited to, values relating to renal function, urine output, blood pressure, catecholamine dosage, combinations of the foregoing, and the like. With regard to body temperature, an appropriate scaling factor is selected from the table 1214 based on instructions 1212.

After the indicator input step 1405, a nutrition rate step 1410 is used to determine a new percentage nutrition rate to be delivered to the patient. In this step, the slide member of the device is moved within the pocket to expose the previous percentage of nutrition dosage that corresponds to the patient in the upper front window of the device, thereby correspondingly exposing new nutrition dosages 1138 (FIG. 12) in the lower front window of the device. The new nutrition dosages exposed in the lower front window are read relative to the current blood glucose values in table 1218 and table 1220.

Once the new nutrition dosages are determined in the nutrition rate step 1410, a recommended absolute nutrition rate is determined in a calculation step 1415. In the calculation step 1415, the product of the target nutrition rate from the target nutrition rate calculation step 1402, the scaling factor selected from table 1214, and the new percentage nutrition rate from the nutrition rate step 1410 is taken.

In an incorporation step 1420, the clinician then references the recommended absolute nutrition rate from the calculation step 1415 and incorporates this value into a therapy decision. Additional changes (including, but not limited to, increase or decrease for minimum or maximum fluid required, increase or decrease to compensate for needed amino acids, increase or decrease for minimum or maximum protein required) can be made as required. Other changes can be made subject to occurrence of diarrhea, abdominal distention, nausea or vomiting or due to high gastric residual volume, or any observed electrolyte abnormalities.

In a delivery step 1425, the absolute nutrition rate based on the therapy decision made pursuant to the incorporation step 1420 is delivered to the patient. A repeat step 1430 provides for control of a loop over selected time intervals (for example, every 1-6 hours), thereby ensuring a consistent therapy and level of care.

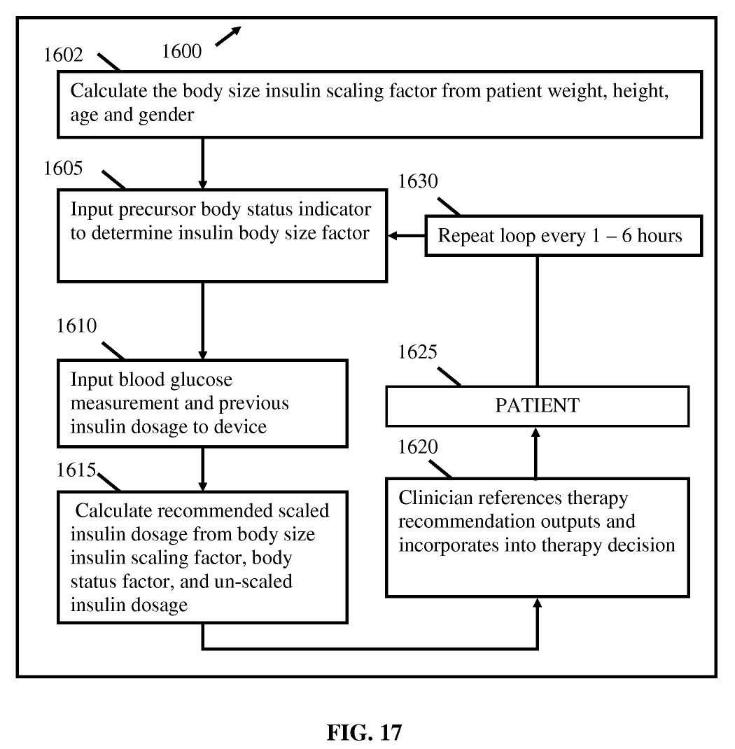

Referring now to FIG. 17, one exemplary method of using the device to determine a recommended insulin dosage for the critically ill and/or diabetic patient is shown generally at 1600 and is hereinafter referred to as “insulin dosage method 1600.” When titrating an insulin dosage utilizing the device in the insulin dosage method, the initial step is typically the calculation of the insulin scaling factor in an insulin scaling factor step 1602. In this step, the clinician determines the appropriate values enumerated in the table 1210 (FIG. 13) that correspond to the patient age, weight, height, and gender and utilizes these values to calculate the body size insulin scaling factor according to equation 1308 (FIG. 13).

In an indicator input step 1605, precursor status indicators are incorporated to assess the current status of the patient. These indicators are real-time parameters that are indicative of the patient's metabolism. Although body temperature is used as the precursor status indicator in the indicator input step 1605, the present invention is not limited in this regard, and other precursor status indicators may be used. Other precursor status indicators that may be input include, but are not limited to, values relating to renal function, urine output, blood pressure, catecholamine dosage, combinations of the foregoing, and the like. With regard to body temperature, the clinician references the table 1314 to select an appropriate scaling factor based on the current body temperature of the patient.

After the indicator input step 1605, an unscaled insulin dosage step 1610 is executed to determine the unscaled insulin dosage to be delivered to the patient. In this step, the slide member of the device is moved within the pocket to expose the current glucose level (shown at 1140 in FIG. 15) in the range that corresponds to the patient and is shown in the upper back window of the device, thereby exposing the two columns of values in the lower back window of the device that correlate to the unscaled insulin dosages (shown at 1142 in FIG. 15). These unscaled insulin dosages exposed in the lower back window are read relative to the first and second insulin dosage value tables (shown at 1318 and 1320 in FIG. 13) located on opposing lateral sides of the lower back window. The clinician reads the unscaled insulin dosage that corresponds to the appropriate value in the first or second insulin dosage value table.

A recommended scaled insulin dosage is then determined in a calculation step 1615. In the calculation step 1615, a recommended scaled insulin dosage is calculated by taking the product of the insulin scaling factor from the insulin scaling factor step 1602, the precursor status indicator from the indicator input step 1605, and the unscaled insulin dosage determined from an unscaled insulin dosage step 1610 for a future period of time which in this case is one hour but may be longer or shorter depending on the basic scale calculations and the tightness of control need for a patient.

In an incorporation step 1620, the clinician then references the recommended scaled insulin dosage from the calculation step 1615 and incorporates this value into the therapy decision. Additional changes (including, but not limited to, increase or decrease for minimum or maximum fluid required, increase or decrease to compensate for needed amino acids, increase or decrease for minimum or maximum protein required) can be made as needed. Other changes can also be made subject to occurrences of diarrhea, abdominal distention, or nausea or vomiting, or due to high gastric residual volume or any observed electrolyte abnormalities.

In a delivery step 1625, the recommended scaled insulin dosage based on the therapy decision made pursuant to the incorporation step 1620 is delivered to the patient for the upcoming time period. A repeat step 1630 provides for control of a loop over selected time intervals (for example, every 1-6 hours), thereby ensuring the consistent therapy and level of care.

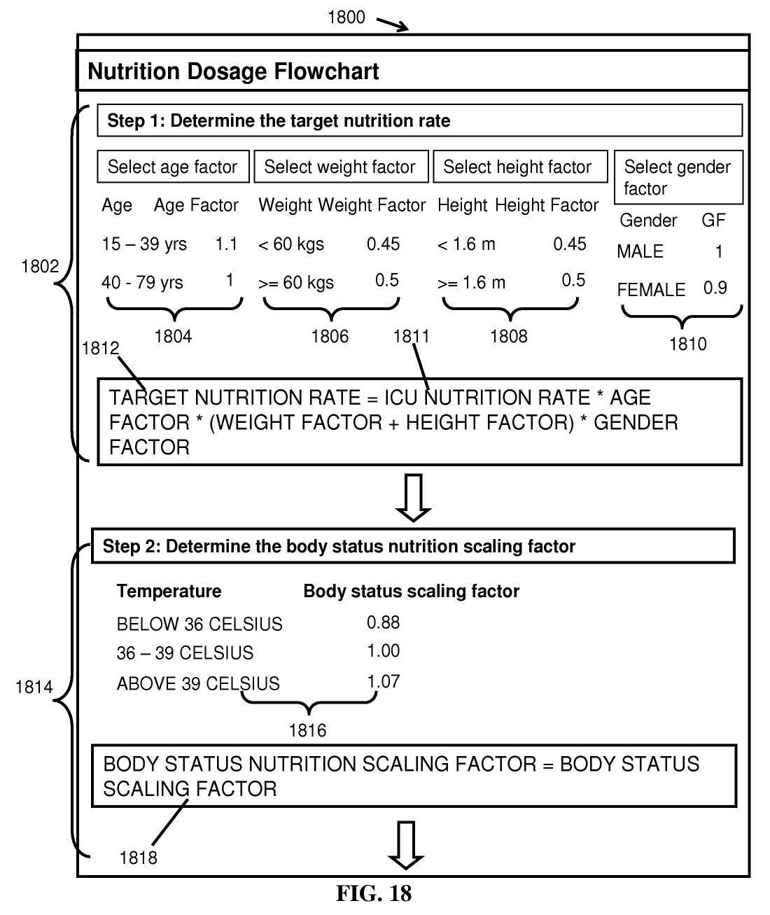

Referring now to FIGS. 18-21, an alternate embodiment of the physiological model of the present invention can be presented via flowcharts in place of the analog computational device. The flowcharts, of which there are four for use with the present invention and which are used to provide nutrition and insulin dosage information to the clinician, are collectively referred to as “flowchart 1800.” The present invention, however, is not limited to the use of four flowcharts, as the information provided herein can be arranged to be displayed on any number of flowcharts. In one embodiment, the flowchart 1800 may be plastic laminated paper that can be written on using erasable markers or the like.

As is shown in FIG. 18, the target nutrition rate 1802 is determined in a first step using tabulated data. The tabulated data includes initial body size parameters. In determining the target nutrition rate 1802, a clinician selects the appropriate body size parameters from, for example, an age factor table 1804, a weight factor table 1806, a height factor table 1808, a gender factor table 1810, and the input of ICU nutrition rate as variable 1811. Corresponding values from each table and the ICU nutrition rate are entered into a target nutrition rate equation 1812, and the target nutrition rate 1802 is solved for.

In a second step, the clinician incorporates precursor indicators to assess the current body status nutrition scaling factor 1814 of the patient. As shown, the flowchart 1800 utilizes a body temperature value selected from a body temperature table 1816 as the precursor indicator. A body status nutrition scaling factor 1818 is selected from the body temperature table 1816. The present invention is not limited with regard to body temperature, however, as other parameters such as renal function, blood pressure, catecholamine dosage, urine output, and combinations of the foregoing can be used as precursors.

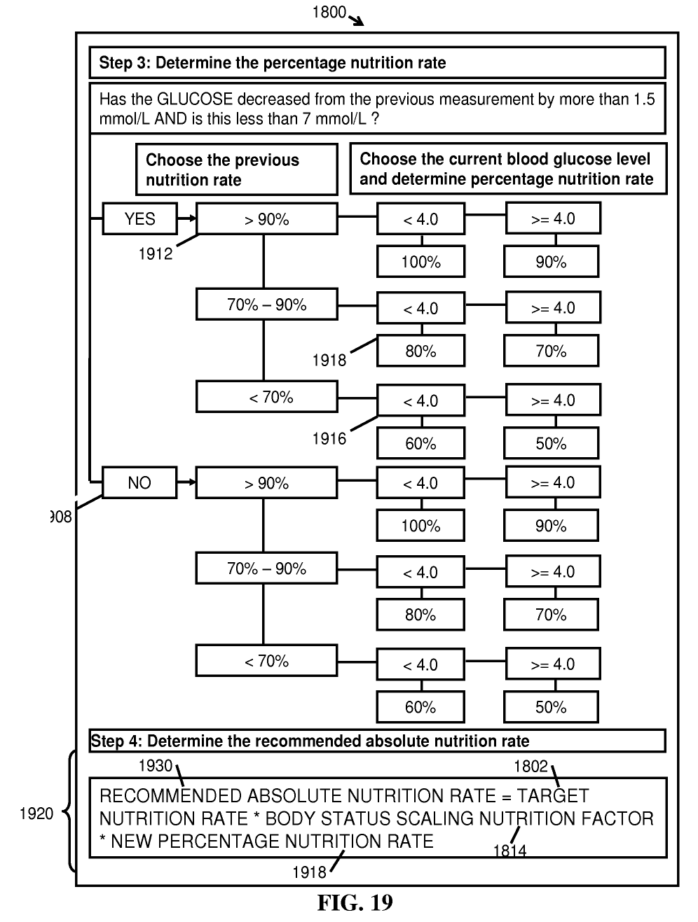

As is shown in FIG. 19, in a third step of the flowchart, the current blood glucose level 1916 and the previous nutrition rate 1912 are used to determine a new percentage nutrition rate 1918 to be delivered to the patient. To determine the new percentage nutrition rate, the clinician is queried as to whether the glucose level of the patient has decreased from the previous measurement by more than a given amount and whether the glucose level falls within a specified range. As shown in the Figure, the clinician is prompted for a yes/no response regarding whether or not the glucose level of the patient has decreased from the previous measurement by more than 1.5 mmol/L and whether or not the value decreased to is less than 7 mmol/L. Depending upon whether the answer to the query is yes or no, the appropriate previous nutrition rate 1912 is selected, and the clinician then selects the appropriate corresponding current blood glucose level 1916. The current blood glucose level 1916 is then used to select the appropriate corresponding new percentage nutrition rate 1918.

Once the target nutrition rate 1802, the current body status nutrition scaling factor 1814, and the new percentage nutrition rate 1918 are determined, the product thereof is taken using equation 1920 in a fourth step to determine the recommended absolute nutrition rate 1930 for a future period of time (which in this case is one hour but may be longer or shorter depending on the basic scale calculations). Equation 1920 is the target rate times the nutrition scaling factor times the percentage output from the algorithm. The clinician can then make additional changes as desired and deliver the therapy to the patient repeating the calculations therefore as necessary. Changes include, but are not limited to, increases or decreases for minimum or maximum fluid required, increases or decreases to compensate for needed amino acids, and increases or decreases for minimum or maximum protein required. Additional changes can be made subject to occurrence of diarrhea, abdominal distention, nausea or vomiting or due to high gastric residual volume or any observed electrolyte abnormalities.

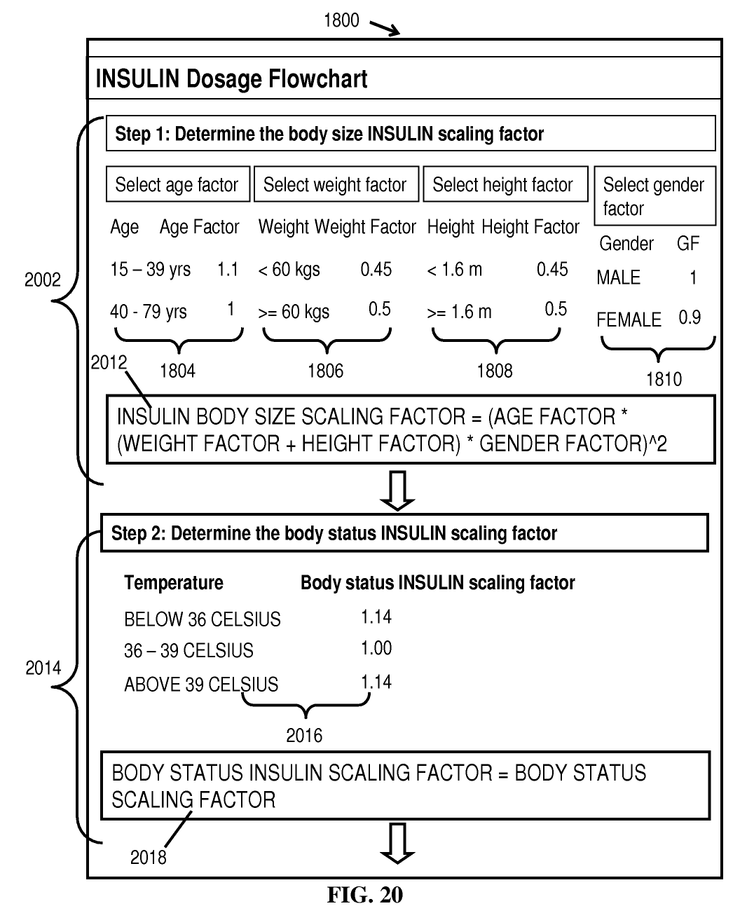

As is shown in FIG. 20, in an effort to ultimately titrate a recommended insulin dosage, the body size insulin scaling factor 2002 is calculated in a first step. The body size insulin scaling factor 2002 is determined from the same tabulated data that is used to calculate the target nutrition rate. The clinician accordingly selects the appropriate patient specific parameters from, for example, the age factor table 1804, the weight factor table 1806, the height factor table 1808, and the gender factor table 1810. These factors are then manipulated according to the equation 2012 to yield the body size insulin scaling factor 2002.

In a second step, the body status insulin scaling factor 2014 is determined via one or more precursor indicators. In the embodiment shown, the flowchart 1800 utilizes a body temperature value selected from a body temperature table 2016 as the precursor indicator. The present invention is not limited in this regard, however, as other parameters such as renal function, blood pressure, catecholamine dosage, urine output, and combinations of the foregoing can be used as precursors.

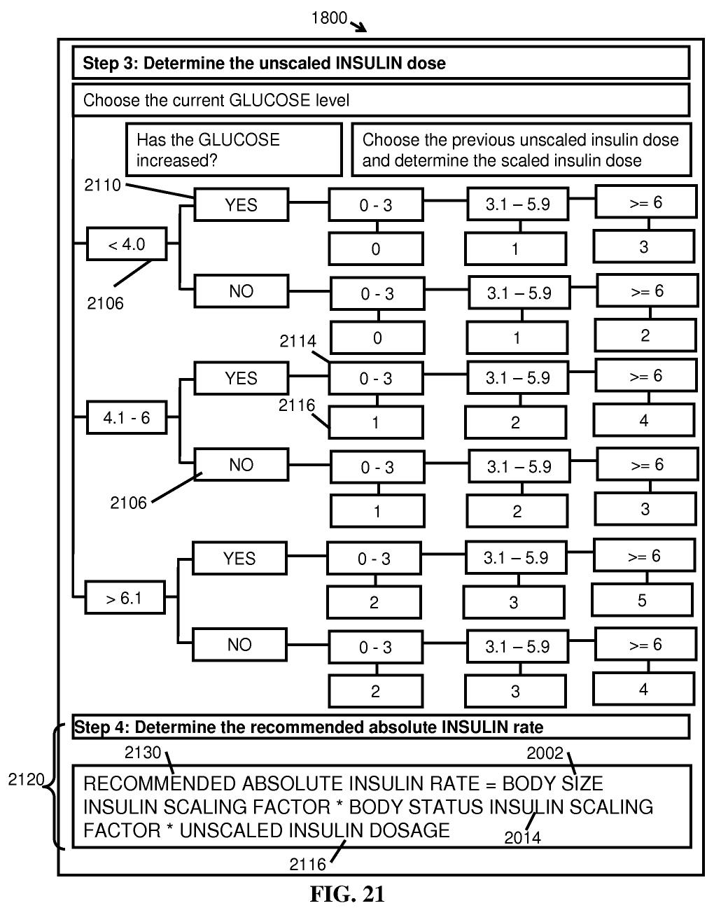

In a third step as shown in FIG. 21, the current blood glucose level 2106 and the previous insulin rate 2114 are used to determine an unscaled insulin dosage 2116 to be delivered to the patient. To determine the unscaled insulin dosage 2116, the clinician is selects the current blood glucose level as indicated in the flowchart 1800. The clinician then determines if the current blood glucose level 2106 has increased relative to the immediately prior blood glucose level and selects a yes or no answer 2110. In the choices for either the yes or no answer 2110, the clinician then selects the previous insulin rate 2114 and the associated unscaled insulin dosage 2116 to be delivered to the patient. For example, if the current blood glucose level of the patient is 7.2 U/hr and this level has increased from the previous sampling, then the clinician would select “yes” and the previous blood glucose level from the choices enumerated. If the previous blood glucose level was 4 U/hr, then the newly determined unscaled insulin dosage 2116 will be 5 U/hr.

Once the body size insulin scaling factor 2002, the body status insulin scaling factor 2014, and the unscaled insulin dosage 2116 are determined, the product thereof is taken using equation 2120 in a fourth step to produce the recommended scaled insulin dosage 2130 for a future period of time (which in this case is one hour but may be longer or shorter depending on the basic scale calculations). This value is incorporated into the therapy decision made by the clinician. The clinician can then make additional changes as desired and deliver the therapy to the patient repeating the calculations therefore as necessary.

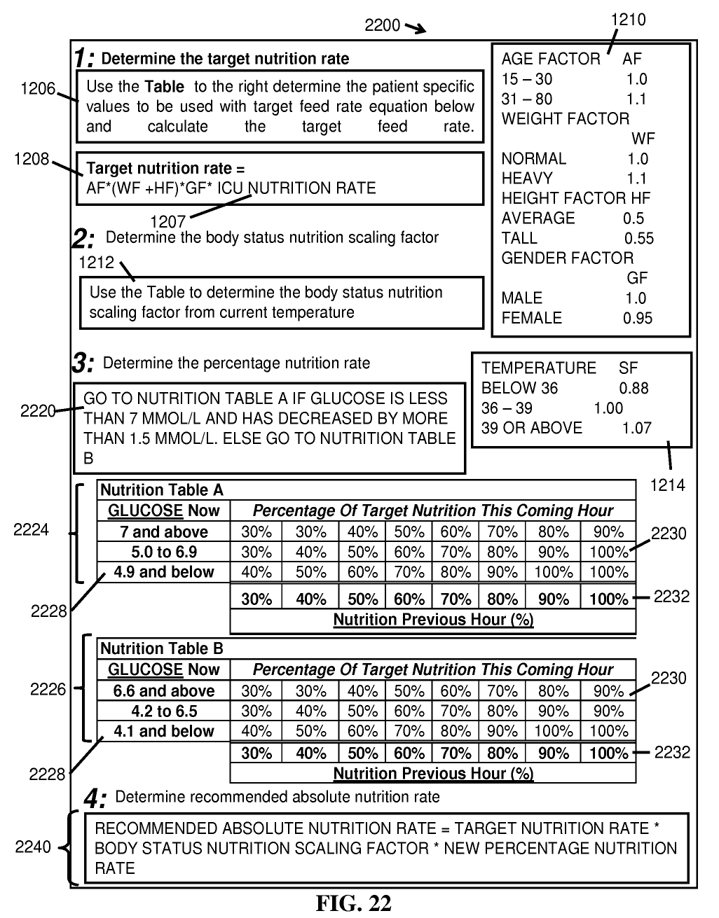

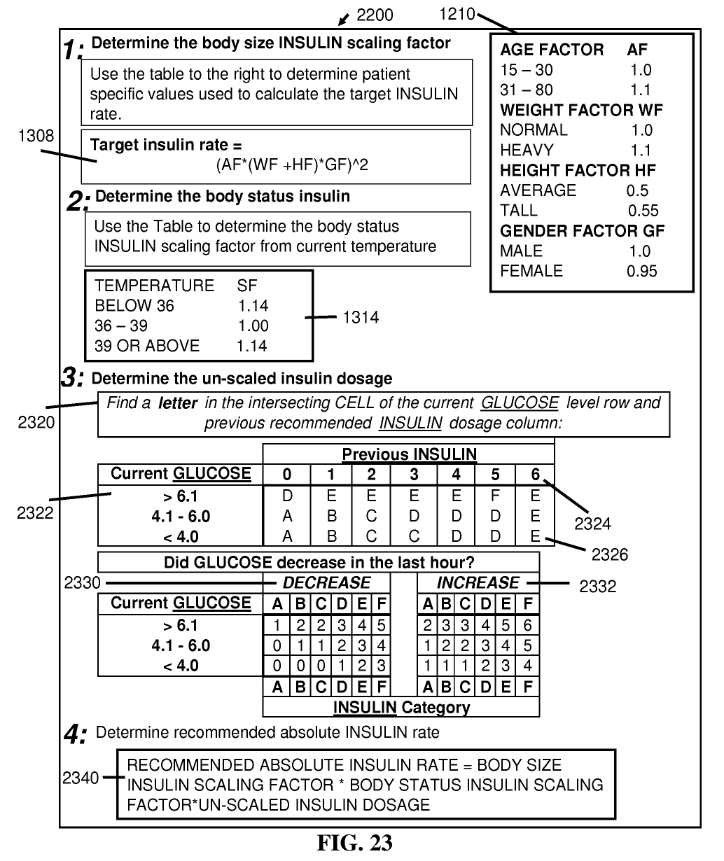

Referring now to FIGS. 22 and 23, another alternate embodiment of the physiological model of the present invention can be presented via tables in place of the analog computational device and the flowcharts. The tables, of which there are two for use with the present invention and which are used to provide nutrition and insulin dosage information to the clinician, are collectively referred to as “tables 2200.” The present invention, however, is not limited to the use of two tables, as the information provided herein can be arranged to be displayed on any number of tables.

As is shown in FIG. 22, in using the tables 2200, in a first step the clinician calculates the target nutrition rate for a patient pursuant to instructions from a table 1206, inputting the ICU nutrition rate 1207, and using the body size parameters provided in the table 1210 and the target nutrition rate equation 1208. The target nutrition rate may be calculated in any suitable volume as a function of time, such as ml/hr.

In a second step, the tables 2200 also include instructions 1212, which provide instructions for determining the body status nutrition scaling factor from the current body temperature of the patient using the table 1214. The table 1214 provides a range of body temperature values and associated scaling factors. The clinician references the table 1214 to select the appropriate scaling factor based on the current body temperature of the patient. The present invention is not limited in this regard, however, as other parameters such as renal function, blood pressure, catecholamine dosage, urine output, and combinations of the foregoing can be used as precursors.

In a third step, the current blood glucose level and the previous nutrition rate are used to determine a new percentage nutrition rate to be delivered to the patient. To determine the new percentage nutrition rate, the clinician answers a query 2220 and assesses whether or not the glucose level of the patient has decreased from the previous measurement by more than a given amount and whether the glucose level falls within a specified range. As shown in the Figure, the clinician is prompted for a yes/no response regarding whether or not the glucose level of the patient has decreased from the previous measurement by more than 1.5 mmol/L and whether or not the value decreased to is less than 7 mmol/L. Depending upon whether the answer to the query is yes or no, the clinician utilizes either table 2224 or table 2226 to determine an appropriate corresponding current blood glucose level 2228. Each current blood glucose level 2228 is then used to select an appropriate corresponding new percentage nutrition rate 2230 (from the cell of the table that intersects the current blood glucose level 2228 and the previous nutrition rate 2232.) Once the target nutrition rate, the current body status nutrition scaling factor, and the new percentage nutrition rate are determined, the product thereof is taken in a fourth step using equation 2240 to determine the recommended absolute nutrition rate for a future period of time (which in this case is one hour but may be longer or shorter depending on the basic scale calculations). The clinician can then make additional changes as desired and deliver the therapy to the patient repeating the calculations therefore as necessary.

As is shown in FIG. 23, the first step in titrating the recommended insulin dosage using the tables 2200 is to calculate the body size insulin scaling factor. In this step, the clinician determines the appropriate values enumerated in the table 1210 that correspond to the patient age, weight, height, and gender and utilizes these values to calculate the body size insulin scaling factor from equation 1308.

In a second step, the body status insulin scaling factor is determined via one or more precursor indicators. In the embodiment shown, the tables 2200 utilize a body temperature value selected from the body temperature table 1314 as the precursor indicator. With regard to body temperature, the clinician references the table 1314 to select the appropriate scaling factor based on the current body temperature of the patient. Although body temperature is used as the precursor status indicator, the present invention is not limited in this regard, and other precursor status indicators may be used. Other precursor status indicators that may be input include, but are not limited to, values relating to renal function, urine output, blood pressure, catecholamine dosage, combinations of the foregoing, and the like.

In a third step, the current blood glucose level and the previous insulin rate are used to determine an unscaled insulin dosage to be delivered to the patient further to instructions 2320. To determine the unscaled insulin dosage, the clinician is selects the current glucose level as indicated in the column 2322. The clinician then locates the intersecting cell of the current glucose level from column 2322 and the previous unscaled insulin dosage row 2324 to find a corresponding category identifier 2326 (as shown, the category identifiers are A, B, C, D, and E), which is then used to determine the unscaled insulin dosage from either table 2330 or table 2332. Table 2330 is referenced when the current blood glucose level has decreased, and table 2332 is referenced when the current blood glucose level has not decreased. The category identifier 2326 is then read across either table 2330 or table 2332 to the corresponding unscaled insulin dosage. For example, if the current glucose level is determined to be 5.4 U/hr and the previous insulin rate for the previous hour was 0, then the intersecting cell would have a category identifier of B. If the current blood glucose level of the patient did not decrease from the previous hour, the unscaled insulin dosage as determined from the cell intersecting the current glucose level value and the category identifier B column (in table 2332) is 2.

Once the body size insulin scaling factor, the body status insulin scaling factor, and the unscaled insulin dosage are determined, the product thereof is taken using equation 2340 in a fourth step to produce the recommended scaled insulin dosage for a future period of time (which in this case is one hour but may be longer or shorter depending on the basic scale calculations). This value is incorporated into the therapy decision made by the clinician. The clinician can then make additional changes as desired and deliver the therapy to the patient repeating the calculations therefore as necessary.

The information in tables 2330 and 2332 could be stored electronically and manipulated in a manner consistent therewith to allow a clinician or healthcare worker to view the relevant information, thereby allowing healthcare decisions to be made. The information may be menu driven using the appropriate visual programming algorithms, graphics, and software (for example, as in the computer graphic embodiments of FIGS. 7 and 8).

In another embodiment, the analog computational device is made up of at least two circular members having a center, a radius, and a viewing panel (pie-shaped or wedge-shaped) mounted on opposing sides of a rectangular base. The circular members have coinciding centers and are rotatable about those centers. One side of the device is used for calculating an insulin dosage recommendation from primary inputs such as renal function, age, gender, weight, height, body temperature, menstrual cycle, APACHE II, and SAPS II and secondary inputs such as blood glucose measurements and previous insulin dosage. The opposing side is used for calculating a nutritional dosage recommendation from primary inputs such as renal function, age, gender, weight, height, body temperature, menstrual cycle, APACHE II, and SAPS II and secondary inputs such as blood glucose measurements and previous enteral or parenteral dosage.

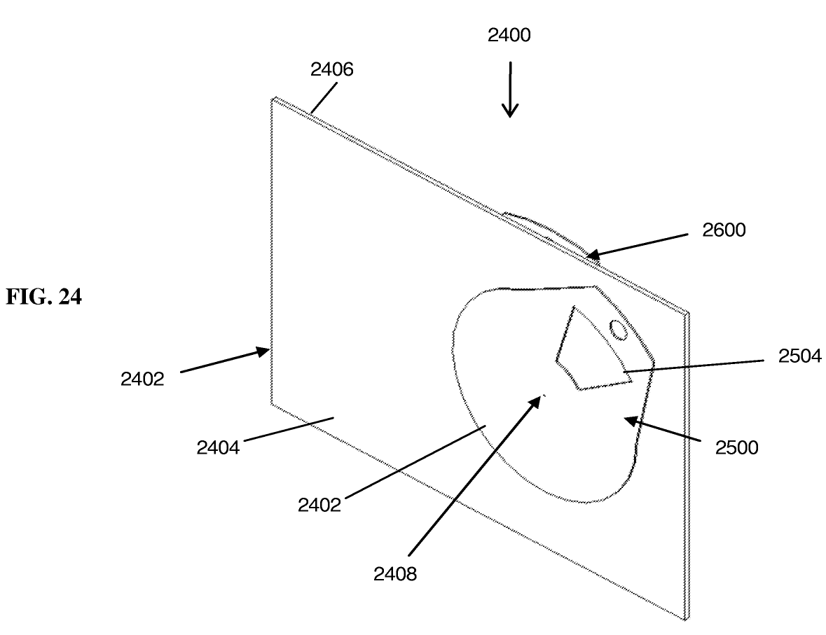

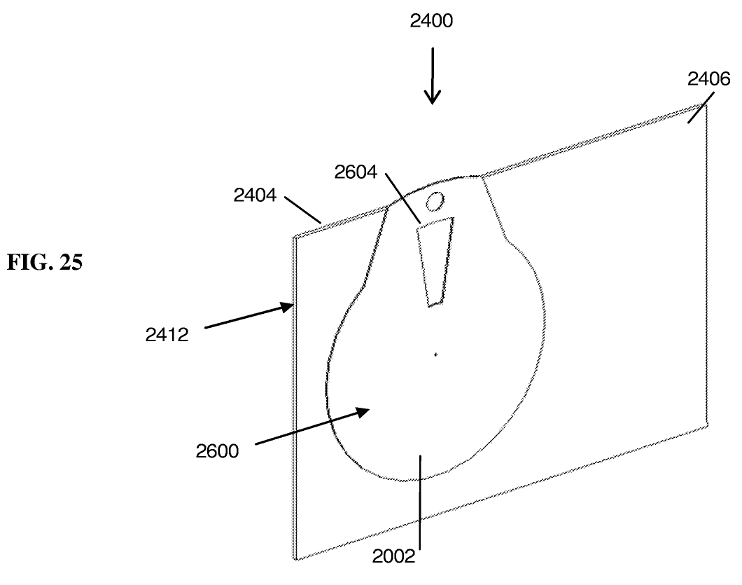

As shown in FIGS. 24 and 25, another embodiment of the analog computational device of the present invention is made up of at least two circular members having a center, a radius, and a viewing panel (pie-shaped or wedge-shaped) mounted on opposing sides of a rectangular base. This analog computational device is shown generally at 2400 and is hereinafter referred to as “device 2400.” The circular members have coinciding centers and are rotatable about those centers. The present invention is not limited in this regard, and other configurations of the device 2400 are within the scope of the present invention.

Device 2400 includes a base 2402 and two circular members 2500 and 2600. Circular member 2500 and circular member 2600 are positioned concentrically and fastened to base 2402 using any suitable means permitting free rotation. In one embodiment, the circular members 2500 and 2600 are fastened to base 2402 by a rivet 2408. Base 2402 is defined by a front panel 2404 and a back panel 2406.

The front panel 2404 and the back panel 2406 may be connected along one or more opposing edges thereof using any suitable means. Materials from which the front panel 2402 and the back panel 2406 can be fabricated include, but are not limited to, paper (for example cardstock), plastic, combinations of the foregoing materials, and the like.

Circular member 2500 is defined at least in part by a visible front panel 2502. As can be seen in FIG. 24, the front panel 2502 includes a front window 2504. Circular member 2600 is defined at least in part by a visible front panel 2602, and as can be seen in FIG. 25, the front panel 2602 includes a front window 2604. The window 2504 and window 2604 each enable data printed on member 2402 to be viewed through the respective windows.

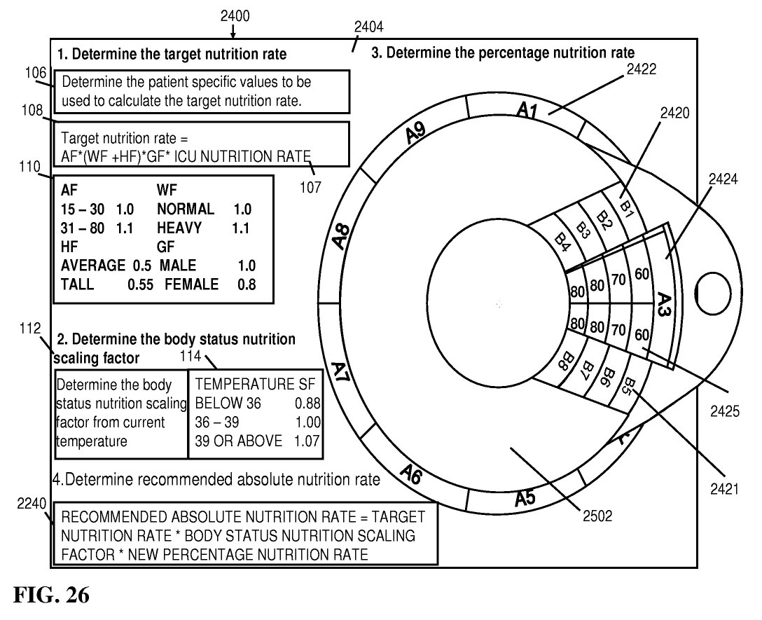

Referring now to FIG. 26, instructional text and calculating information is printed on the front panel 2404 of the device 2400. In one embodiment, such text and information includes instructions 106 that provide for calculating a target nutrition rate for a patient from body size and age parameters provided in a table 110, inputting variable 107 the ICU nutrition rate for specific intensive care unit in which the device is being used, and using a target nutrition rate equation 108. These body size and age parameters are patient specific physical attributes. The target nutrition rate may be calculated from these parameters in any suitable volume as a function of time, such as milliliters per hour (ml/hr).

Instructions 112 also provide for determining a body status nutrition scaling factor from the current body temperature of the patient using a table 114. The table 114 provides a range of body temperature values and associated scaling factors. The clinician references the table 114 to select the appropriate scaling factor based on the current body temperature of the patient.

The present invention is not limited to the printing of table 110 or table 114 onto the front panel 2404, however, as the information provided therein may be provided elsewhere.

On the nutrition dosage side, preferably, a series of blood glucose ranges (B1-B8) 2420 is found on the front panel 2404 along a radius adjacent to the viewing window 2504. The viewing panel may be an open window-like area or a transparent or translucent material such as cellophane. The calculator also includes a circular design 2422 printed onto the front panel 2404 of the rectangular base 2402 similarly having a center and a radius larger than the radius of the first circular member. A number of nutrition dosage values, each array having a number of nutrition dosage values, are positioned on the back circular member in an area and position such that the values are visible in the viewing panel those values correspond to the previous blood glucose ranges 2420 and 2421 on the adjacent viewing panel. The displayed nutrition dosage values further correspond to a previous nutrition dosage positioned on the exterior periphery of the printed circular design (A1-A9).

A previous nutrition dosage value 2424 is viewable through the front window current nutrition rates 2425 are additionally viewable through the front window. A first current blood glucose value table 2420 and a second current blood glucose value table 2421 are located on opposing lateral sides of the window on the front panel 2502. The first current blood glucose value table 2420 is indicative of the blood glucose level of the patient when the previous blood glucose level has not decreased by more than a preselected amount over a given time period. The second current blood glucose value table 2421 is indicative of the blood glucose level of the patient when the previous blood glucose level has decreased by more than a preselected amount over the same given time period. In one embodiment of the present invention, the first current blood glucose value table 2420 is used when the previous blood glucose level has not decreased by more than 1.5 mmol/L over the past hour, and the second current blood glucose value table 2421 is used when the previous blood glucose level has decreased by more than 1.5 mmol/L over the past hour.

In one embodiment of the present invention, the first current blood glucose value table 2420 and the second current blood glucose value table 2421 are each arranged to define four ranges of blood glucose values. If the patient has blood glucose measurements below 4 mmol/L, use of the device 2400 will allow a clinician to calculate an increase in the current percentage nutrition rate. In addition, even if the patient is in the target 4-6 mmol/L range, an increase in feed in an effort to bring the nutrition level up to 100% of the target nutrition rate can be calculated. Depending upon the previous and current nutrition dosages, however, only a percent of the target nutrition rate may be calculated and recommended to be administered to the patient for a future period of time which in this case is one hour but may be longer or shorter depending on the basic scale calculations and the tightness of control required. Typically, a lower limit of about 30% of the target nutrition rate is imposed to ensure that the nutrition rates administered are within safe, acceptable limits.

Once the target nutrition rate, the current body status nutrition scaling factor, and the new percentage nutrition rate are determined, the product thereof is taken in a fourth step using equation 2240 to determine the recommended absolute nutrition rate for a future period of time which in this case is one hour but may be longer or shorter depending on the basic scale calculations. The clinician can then make additional changes as desired and deliver the therapy to the patient repeating the calculations therefore as necessary. For example, the clinician can administer effective amounts of nutrition and insulin based on the recommended nutrition rate and the recommended insulin dosage to control the patient's blood glucose level. In doing so, the incidence of ventilator-induced pneumonia in the patient is reduced or prevented altogether. When using the invention the incidence of ventilator-induced pneumonia in cohorts treated with the invention is reduced when compared to prior cohorts not treated by the invention by 15%. This is a significant reduction.

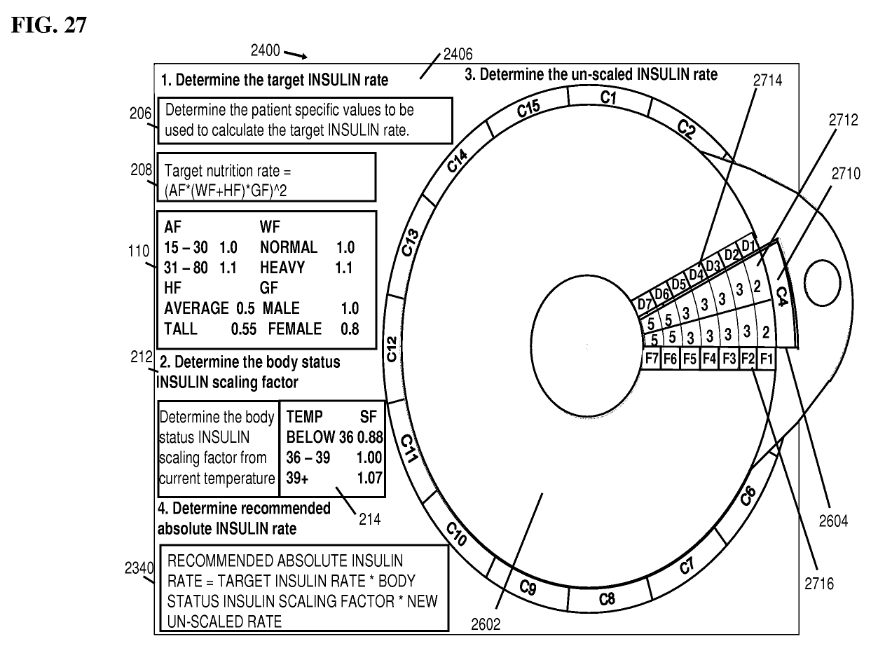

As shown in FIG. 27, instructional text and calculating information is also printed on the back panel 2406 of the device 2400. In one embodiment, this text and information includes instructions 206 that provide for calculating a body size insulin scaling factor from a body size insulin scaling factor equation 208 for the patient from body size and parameters provided in the table 110.

Instructions 212 also provide for determining a body status insulin scaling factor from the current body temperature of the patient using a table 214. The table 214 provides a range of body temperature values and associated scaling factors.

The present invention is not limited to the printing of table 110 or table 214 onto the back panel 2406, however, as the information provided therein may be provided elsewhere.

A current blood glucose level 2710 is viewable through the upper back window 2004, and an unscaled insulin dosage 2712 is viewable through the window 2604. A first insulin dosage value table 2714 and a second insulin dosage value table 2716 are located on opposing sides of the window 2604 in the back panel 2602. The first insulin dosage value table 2714 is indicative of the insulin dosage administered to the patient when the blood glucose level has increased by more than a preselected amount over a given time period. The second insulin dosage value table 2716 is indicative of the insulin dosage administered to the patient when the blood glucose level has decreased by more than a preselected amount over the same given time period which in this case is one hour but may be longer or shorter depending on the basic scale calculations.

In one embodiment of the present invention, the first insulin dosage value table 2714 and the second insulin dosage value table 2716 are each arranged to define seven insulin dosage values, namely, zero through 6 units per hour (U/hr) in 1 U/hr increments. The insulin dosage values are capped at 6 U/hr because any insulin beyond this amount is considered to be ineffective due to saturation of the insulin effect.

Once the body size insulin scaling factor, the body status insulin scaling factor, and the un-scaled insulin dosage are determined, the product thereof is taken using equation 2340 in a fourth step to produce the recommended scaled insulin dosage for a future period of time which in this case is one hour but may be longer or shorter depending on the basic scale calculations. This value is incorporated into the therapy decision made by the clinician. The clinician can then make additional changes as desired and deliver the therapy to the patient repeating the calculations therefore as necessary.

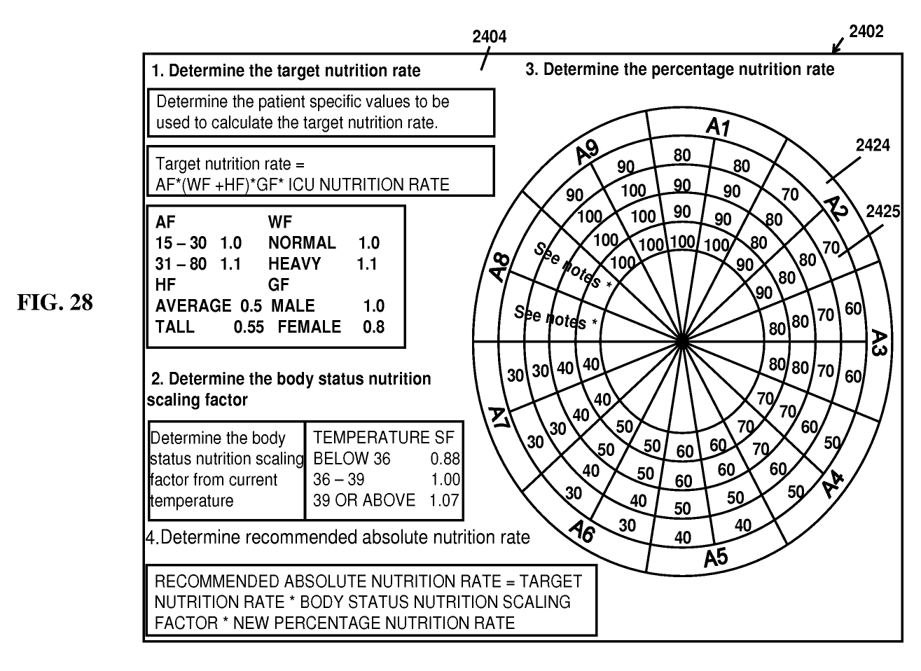

As shown in FIG. 28, the front face 2404 of base 2402 includes data indicative of the previous nutrition dosages 2424 as well as data indicative of current nutrition dosages 2425. The previous percentages of nutrition dosages 2424 and the current nutrition dosages 2425 are arranged into an array such that particular previous percentages correspond to particular current dosages.

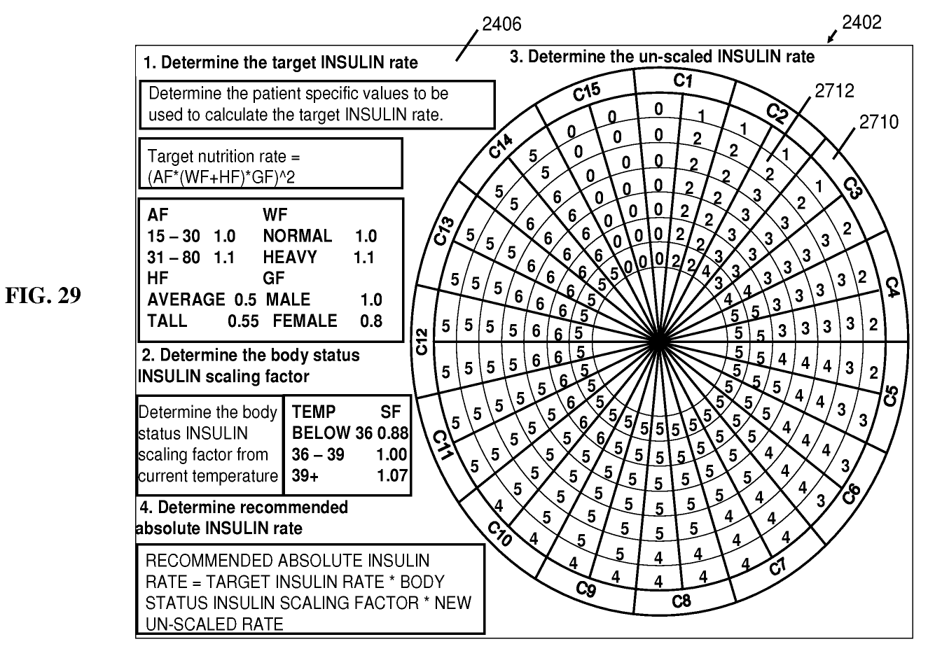

As shown in FIG. 29, the back face 2406 of the base 2402 includes data indicative of the current glucose level 2710 as a range and the unscaled insulin dosages 2712. The values for the current glucose levels 2710 and the unscaled insulin dosages 2712 are arranged into an array such that particular current glucose levels correspond to particular unscaled insulin dosages.

The present invention as defined in any of the above embodiments can be used not only for patients requiring critical care but also by patients with diabetic conditions who desire tight control of their care. By monitoring and accounting for the precursor indicators, the patient with a diabetic condition can maintain a suitable glucose level within a range that is conducive to their metabolic conditions. The present invention may be better understood in view of the following Examples.

The present invention as defined in any of the above embodiments can be used not only for patients requiring critical care but also by patients with diabetic conditions who desire tight control of their care. By monitoring and accounting for the precursor indicators, the patient with a diabetic condition can maintain a suitable glucose level within a range that is conducive to their metabolic conditions. The present invention may be better understood in view of the following examples.

### Example 1

### Development of Glucose-Insulin Kinetic Model

Initial efforts during the development of the manual calculation approach described herein commenced with critically ill patients undergoing intensive care therapy. The patients studied had high levels of insulin resistance and impaired glucose metabolism associated with severe illness. The data collected on these patients led to the development of an initial glucose-insulin system model based on a physiological insulin model. This model is shown below:

$$\begin{matrix}\dot{G} = - {p}_{G} G - {S}_{I} ( G + {G}_{E} ) \frac{Q}{1 + {α}_{G} Q} + P ( t ) & ( 1 ) \\ \dot{Q} = - kI + kQ & ( 2 ) \\ \dot{I} = - \frac{nI}{1 + {α}_{1} I} + \frac{{u}_{ex}}{V} & ( 3 )\end{matrix}$$

### Glucose-Insulin Kinetic Model

Where the inputs and outputs are:
G(t)=Plasma glucose above equilibrium glucose concentration GE
I(t)=Plasma insulin from exogenous input Uex(t)
Q(t)=The effect of infused insulin
k=The effective half-life parameter of insulin
p_{G}=Patient clearance of glucose
S_{I}=Insulin Sensitivity
V=Insulin distribution volume
n=Constant 1st order decay rate of insulin
P(t)=Total plasma glucose input
α_{G}=Saturation parameter of insulin-stimulated glucose uptake
α_{I}=Saturation parameter of plasma insulin removal

This kinetic model functions as a closely approximated surrogate for a human body. Inputs to the kinetic model included the times and amounts of nutrition and insulin taken by the diabetic patient. These inputs were used in the kinetic model to solve for an output blood glucose level, which were then used to describe population human metabolic behavior and to account for intra-patient variability and evolving metabolic conditions.

This physiologically verified mathematical model was used to perform virtual patient trials in order to design and develop decision support systems before clinical implementations. By taking this approach, a much wider variety of methods and systems was tested, thereby allowing a more complete and more rapid development process to occur.

The system was used in simulation scenarios as a vehicle for experimentation. Because this system was capable of accurately capturing the behavior and dynamics of a human glucose metabolism system, it was used to receive multiple inputs and generate a single output therefrom. Utilizing these systems in this manner allowed risks and costs associated with actual clinical trials to be eliminated or at least minimized.

Given that the behaviors of metabolic systems of various individuals deviate, however, various additional factors can be considered with regard to the kinetic model. For example, factors such as the extents of particular disease states, physical characteristics (e.g., obesity, fitness, body temperature, and the like), and genetic factors such as predispositions to particular types of diseases, obesity, and the like can affect human metabolism. To account for such deviation in the behaviors of metabolic systems, the kinetic model incorporated a tuning variable that was operable with respect to inputs that can be considered to be non-primary factors, thereby allowing the kinetic model to adapt to and describe diseased metabolic states. This tuning variable, which is embodied as the insulin sensitivity (S_{I}), describes the current ability of the body to utilize insulin effectively. Insulin sensitivity is a quantitative measure of insulin resistance and is a dynamic physiological parameter and key driver of observed dynamics of the metabolic system for patients undergoing critical care therapy. In critical care therapy, the stress and trauma placed on the body impairs the body's ability to fully utilize insulin. Thus, the insulin sensitivity value of a patient undergoing critical care therapy changes relative to an insulin sensitivity value of a healthy person or even relative to an insulin sensitivity value of a person having compromised health but not undergoing critical care therapy.

Although a future value of insulin sensitivity is difficult to predict, histories can be determined from retrospective patient data and utilized to generate insulin sensitivity “profiles.” To generate an insulin sensitivity profile, data relating to the nutrition and insulin habits of the patient (including blood glucose levels) are collected. The profile can then be used to enable the kinetic model to respond specifically to the needs dictated by the metabolic issues of the patient.

Accordingly, each insulin sensitivity profile becomes a “virtual patient” and can be used in combination with the kinetic model, a nutrition regime, and an insulin regime to determine a resulting set of blood glucose levels. The nutrition and the insulin regimes are designed to reduce high blood glucose levels and maximize the time that the values of the blood glucose levels remain between about 4 mmol/L to about 6 mmol/L. By using the virtual patient approach, patient care is simulated. The results of such simulations can be correlated to the control of blood glucose levels in the field, thereby providing proof of concept and allowing extensive experimentation to be performed with no compromise in patient health.

### Example 2

### Insulin-Only Modulation Strategy

Initially, an insulin-only approach was employed to reduce hyperglycemia in the intensive care unit. Protocols for insulin-mediated glycemic control using model-based methods were developed.

To verify assumptions regarding the employed approaches, clinical trials were performed in which an insulin bolus-based adaptive control protocol was employed. In this protocol, blood glucose level in a patient was measured every 30 minutes, and this value was used to calculate an insulin dosage amount administered intravenously to the patient to reduce blood glucose levels. This control loop was repeated every 30 minutes over the length of the trial. One observation was that insulin-based protocols were severely challenged in the administrations of critical care therapies where insulin resistances were often significantly elevated. In these conditions, the insulin effect saturates at approximately 5-6 U/hr and the body cannot utilize any additional insulin above this amount to reduce hyperglycemia. This saturation phenomenon limits the level of control achievable with insulin alone. Trial results using the insulin only approach are presented in Table 12 below:

TABLE 12
Glycemic control results from an insulin-only modulation strategy

| | |
|---|---|
| % Time in 4-6.0 mmol/L | 25.22% |
| % Time in 4-7.75 mmol/L | 65.10% |
| Average insulin rate (U/hr) | 5.1 |

The insulin-only approach only achieved approximately 25% of all blood glucose levels in the 4-6.0 mmol/L range. Additionally, approximately 65% of blood glucose measurements were in the 4-7.75 mmol/L range. As shown in Table 12, the average insulin rate of the insulin-only approach was 5.1 U/hr. Given that the insulin effect saturates at 5-6 U/hr, it was clear that additional control means were needed to improve glycemic control. In critical care, the time in the 4-6.0 mmol/L band should be maximized. Furthermore, it was determined that if the patient's blood glucose can be kept in this range as long as possible, mortality and morbidity were significantly reduced in this setting. This study did not detect any significant improvement in on neuromuscular complications, ventilator dependency and a reduction in ventilator-induced pneumonia.

### Example 3

### Insulin and Nutrition Modulation Strategy

To overcome the limitations of insulin-only approaches, models were developed using both exogenous insulin and nutrition inputs. Additionally, modifying the carbohydrate intake allows another avenue of reducing blood glucose levels and hence glycemic reduction can be effected by changing the exogenous nutrition inputs. Lower glucose nutrition alone in critical care was shown to result in reductions in average blood glucose levels and improved clinical outcome. By feeding over 66% of the recommended rates of nutrition, it was found that the likelihood of ICU mortality was increased. This suggested that the caloric targets, which were recommended by the American College of Chest Physicians, may be set too high. Additional examples can be found in pediatric and obese subjects. Thus, it was determined that moderate nutrition reductions can improve mortality rates without adversely affecting other clinical outcomes. Additional trials were conducted employing an insulin and nutrition modulation control strategy. The results are shown in Table 13.

TABLE 13
Glycemic control results from an insulin and nutrition modulation strategy

| | |
|---|---|
| % Time in 4-6.0 mmol/L | 47.98% |
| % Time in 4-7.75 mmol/L | 91.12% |
| Average feed rate (mL/hr) | 54.6 |
| Average insulin rate (U/hr) | 3 |

Several important findings became apparent from these results. First, patient nutrition was confirmed as a driver of the metabolic function. Changing the patient nutrition rate greatly improved metabolic control. The time in the 4-6.0 mmol/L band improved significantly to 48%, and the time in the 4-7.75 mmol/L band increased to 91%. Second, the average insulin rate decreased to 3 U/hr. This data disproved the accepted clinical doctrine that simply increasing insulin dosage improved blood glucose control.

Administering the patient more insulin when the blood glucose is high and less insulin when the blood glucose is low is by itself not effective. It is a single input and single output simplification of a very complex multivariable system which does have the ability to control the process. Insulin sensitivity is a major factor in the decision for what action to take. If the patient has low insulin sensitivity, an abundance of insulin can be injected but the reduction in blood glucose will be minimal. Insulin saturates in the body resulting in reduced effect from additional insulin. These trials showed that in cases of low insulin sensitivity altering the nutrition is an effective additional input. However, changing the patient's nutrition level does not have an instant effect as it can take several hours for the patient's body to react to the new nutrition level. Also in the trials it was determined that it is undesirable to change the patient's nutrition too frequently as it results in highly dynamic behaviour. From these conclusions it was determined that there were other dynamic factors that had not been considered.

Two additional sets of clinical trials were performed, each utilizing different computerized insulin and nutrition modulation protocols. Initial trials used a fully computerized protocol that calculated recommended insulin and nutrition dosages from previous insulin and nutrition dosage and measured blood glucose levels. During the study, data was collected that was believed to be precursor indicators of the glucose and insulin utilization process of the patients. A limited number of trials was conducted given that wide-spread implementation of a fully computerized protocol faced significant cost and logistic barriers. In addition, some of the methods employed were not fully developed and needed to incorporate additional parameters to improve the level of blood glucose control. The study did detect a slight improvement in on neuromuscular complications, ventilator dependency and a reduction in ventilator-induced pneumonia. When using the invention the incidence of ventilator-induced pneumonia in cohorts treated with the invention is reduced when compared to prior cohorts not treated by the invention by 4%.

Next, an effort was made to condense all the progress involved in modeling and regulating the blood-glucose system into a set of tables that would be easy for nurses, clinicians, and other practicioners in an ICU to use. It was believed that providing a repeatable and easy to use process would give dependable results. To achieve this, the protocol would have to be able to provide tight control using readily available information. Therefore, a major factor in the initial design of the tables was the amount of information the nurses, clinicians, and other practicioners have available at hand. Also included were the measurements that were regularly and routinely taken in the ICU. Initial development on a tabular system incorporated the same set of inputs as the other and more advanced protocol, namely, insulin dosage history, nutrition dosage history, and blood glucose measurements. The tabular protocols were developed via iterative experimentation on the virtual simulations.

The initial tabular system, which was termed “the SPecialized Relative Insulin Nutrition Tables” or “SPRINT” system, utilized input data of previous insulin and nutrition dosages and blood glucose levels and could generate recommended nutrition and insulin dosage outputs. This approach of modulating both nutrition and insulin was shown to be effective but did not track with our expected results although the tabular instantiation was well received by clinical staff. Further clinical trials were performed using this tabular instantiation of the insulin and nutrition modulation strategy. The study did detect a slight improvement in neuromuscular complications, reduction in new infections, ventilator dependency and a reduction in ventilator-induced pneumonia over patient populations not under glycemic control. When using the invention the incidence of ventilator-induced pneumonia in cohorts treated with the invention is reduced when compared to prior cohorts not treated by the invention by 6%.

However, during the development it became very apparent that these parameters were not the only mechanism at work within the patients. Other factors were offsetting the ability of the algorithm to bring the patient's metabolism into control. Clinical results obtained did not match the predicted outcomes, so it was theorized that the model was not addressing significant inputs to the algorithm. To determine the significance and to identify the factors, a review of the patient data collected was performed, which led to the identification of parameters that were indicative of a patient's health and could affect patients insulin and glucose utilization. This provided insight to the concept that there were other factors involved. These included many factors that were regularly observed and recorded. Such factors included physical parameters such as age, gender, weight, body frame size, body temperature, and body surface area; endogenous glucose clearance; liver function tests including assays for albumin (Alb), alanin transaminase (ALT), aspartate transaminase (AST), alkaline phosphatase (ALP) and total bilirubin (TBIL), gamma glutamyl transpeptidase (GGT), 5′ nucleotidase (5′NTD), coagulation test (e.g. INR), and serum glucose (BG, GLu); presence of sepsis; pregnancy and time of menstrual cycle; renal function as estimated via glomerular filtration rate; diurnal cycles, circadian rhythms, and bio-rhythms; serum creatinine concentration, aminoglycoside dosage, serum aminoglycoside concentration, urea concentration, noradrenaline usage, duration of mechanical ventilation, muscle relaxant usage such as pancuronium and rocuronium, and urine output; ineffective insulin levels; ethnicity; endogenous clearance levels based on lean body mass or other similar levels; ICU insulin sensitivity variability; APACHE II score or similar test results for ICU-level of critical illness; heart rate, systolic pressure, diastolic pressure, pulmonary artery wedge pressure, central venous pressure, mixed venous oxygen saturation, oxygen saturation, tidal volume, inspiratory pressure, positive end expiratory pressure, respiration rate, electroencephalography and bispectral index, patient history, caregiver notes, laboratory reports, venous pressure, and urine output; change in kind of medication, dose of medication, means of administration of medication, and change in caloric intake, BMI, prior history of diabetes, on-admission parameters, reason for ICU admission, APACHE-II on admission, on-admission glycemia, caloric intake on admission, and concomitant medication on admission; medical history, recent medications, current or past surgical procedures, current vital signs, and current medical condition; ECG Sensor and EEG; physical activity, blood glucose data, meal intake, and insulin intake; blood glucose history profile; insulin history; heart rate history; heart rate variability history; EKG; temperature over time; perspiration levels; skin conductivity; HbA1C level and history; CD4 information, viral load information, HIV genotype and phenotype information, hemoglobin information, neuropathy information, neutrophil information, pancreatic function, hepatic function, drug allergy, and intolerance information; size and type of a meal to be ingested, anticipated duration and intensity of exercise; C-peptide concentration; mode of respiratory ventilation, set breathing rate, spontaneous breathing rate, ratio of inspired oxygen; type of nutrition, amount of aspirated nutrition, hourly urine, running total urine; medication history including, but not limited to, antibiotics, cardiac agents, prokinetics, steroids, sedatives, morphine, midazolam, vascoactive drugs, noradrenaline, adrenaline, dobutamine, vasopressin; lab investigation results including blood gases, blood count, white cell count, neturophils, platelets, blood culture, sputum/tracheal aspirate, urine/CSU; blood pH and lactate concentration, urine glucose concentration, and plasma insulin concentration; and diagnosis of diabetes (type-1, type-2, gestational).

The number of metabolic indicators that could be attributed to the inconsistency of the results presented a significant issue. Studies were conducted to determine if a subset of the indicators could suitably indicate insulin and glucose utilization by a patient. As these factors were reviewed, it was found that the list could be shortened to primary factors such as body temperature, renal function, urine output, blood pressure, catecholamine dosage, age, weight, height, and gender to suitably fit the adjustments required for a metabolic control algorithm to make a patient respond according to initial predictions. Incorporating these factors into the therapy support algorithm as scaling factors to modify the therapy recommendations generated from the described algorithm, it was realized that a more effective control of patients could be gained. Furthermore, it was realized that glucose levels could be brought under control more quickly. By incorporating additional clinical markers of body temperature, renal function, urine output, blood pressure, catecholamine dosage, age, weight, height, and gender, it was possible to better assess the current patient state and then optimally titrate therapy dosages to match the demands of the patient's metabolism.

### Example 4

### Modulation Strategy Incorporating Body Parameters

Additional studies were conducted investigating the approach of using age, weight, height, and gender to calculate a target nutrition rate and a body-size insulin scaling factor. All four of these variables are assessed and documented by a clinician upon patient admission to the ICU. The data was readily available to nursing staff and could easily be incorporated into therapy decisions. This approach resulted in a marked improvement in blood glucose control.

The four factors (age, weight, height, and gender) were used in the following formula to calculate a target nutrition rate:

$${P}_{max} = \frac{{A}_{g} ( {W}_{e} + {H}_{e} ) {G}_{en} \times 2000}{24} \qquad ( 4 )$$

where the following variables were used:
Pₘₐₓ=target nutrition rate
A_{g}=age factor
Wₑ=weight factor
Hₑ=height factor
Gₑₙ=gender factor

This is based on the assumption that a larger body frame would have a higher BMR and hence would require a larger carbohydrate intake to maintain normal glucose levels. If the same constant nutrition rate was delivered to all patients, it is assumed that physically smaller patients would struggle to reduce blood glucose levels. Hence an equation was developed to calculate a target nutrition rate from age, weight, height, and gender of an individual to customize nutrition rates to the needs of the patient. Table 14 shows the values of the scaling factors used to calculate the target nutrition value.

TABLE 14
Optimized values used to calculate target nutrition value

| Age | Age Nutrition Scaling Factors |
|---|---|
| 15-39 years | 1.1 |
| 40-59 years | 1 |
| 60-79 years | 0.9 |
| 80+ years | 0.8 |
| Weight | Weight Nutrition Scaling Factors |
| Light | 0.45 |
| Average | 0.5 |
| Heavy | 0.55 |
| Height | Height Nutrition Scaling Factors |
| Short | 0.45 |
| Average | 0.5 |
| Tall | 0.55 |
| Gender | Gender Nutrition Scaling Factors |
| Male | 1 |
| Female | 0.9 |

As a human ages, their BMR gradually decreases. Thus, older patients are assigned lower nutrition scaling factors. In addition, BMR increases with patient size, so heavier and taller patients are assigned higher nutrition scaling factors. Males are known to have higher BMR values than females and hence the gender nutrition scaling factor is 1 for a male and 0.9 for a female.

The nutrition dosage algorithm was rewritten in terms of percentage levels of the target nutrition rate. Accordingly, the same core algorithm was applied to all patients, and the use of the target nutrition rate customized the nutrition levels to patient physical characteristics. The results of trials incorporating the customized nutrition levels are displayed below in table 15.

TABLE 15
Glycemic control results using a target nutrition level determined
from patient age, weight, height, and gender

| | Target Nutrition = | Target Nutrition = |
|---|---|---|
| | 100 mL/hr | patient-specific |
| % Time in 4-6.0 mmol/L | 47.98% | 54.22% |
| % Time in 4-7.75 mmol/L | 91.12% | 91.88% |
| Avg % of goal feed | 54.60% | 62.78% |
| Feed rate (mL/hr) | 54.6 | 46.1 |
| Average insulin rate (U/hr) | 3 | 2.86 |

From these trials it was determined that nutrition should be customized to individual patients. Time in the ideal 4-6.0 mmol/L and 4-7.75 mmol/L band increased from 48% to 54% and 91% to 92%, respectively, when customized nutrition methods were employed. Thus, incorporating patient-specific nutrition administration significantly increases time in the ideal glycemic range of 4-6 mmol/L.

The four factors (age, weight, height, and gender) were used in the following formula to calculate the insulin body size scaling factor:

U_{scale}=(A_{g}(Wₑ+Hₑ)Gₑₙ)² (5)

Where the following variables were used;
U_{scale}=insulin body size scaling factor
A_{g}=age factor
Wₑ=weight factor
Hₑ=height factor
Gₑₙ=gender factor
   The same set of age, weight, height, and gender factors were used with the insulin body size scaling factor for convenience of the nursing staff. Insulin distribution volume increases with patient size; hence the insulin scaling factor increases for males and patients having greater height or weight. Table 16 shows the values of the variables used in conjunction with equation 5.

TABLE 16
Values used to calculate insulin body size scaling factor

| Age | Age Factor |
|---|---|
| 15-39 years | 1.1 |
| 40-59 years | 1 |
| 60-79 years | 0.9 |
| 80+ years | 0.8 |
| Weight | Weight Factor |
| Light | 0.45 |
| Average | 0.5 |
| Heavy | 0.55 |
| Height | Height Factor |
| Short | 0.45 |
| Average | 0.5 |
| Tall | 0.55 |
| Gender | Gender Factor |
| Male | 1 |
| Female | 0.9 |

The insulin scaling factor is incorporated into the insulin dosage algorithm by simply multiplying this factor by the unsealed insulin algorithm output. The single algorithm presented on a single device can still used to calculate the insulin bolus to be delivered to the patient. The unsealed insulin bolus output of the algorithm is multiplied by the body size insulin scaling factor to make the output patient specific. Although the core algorithm can be applied to all patients, the use of the insulin body size scaling factor value customizes the insulin levels to a patient's physical characteristics. Table 6 shows the improvements in glycemic control achieved by utilizing the insulin body size scaling factor.

TABLE 17
Glycemic control results using an insulin body size scaling factor
determined from patient age, weight, height, and gender

| | No scaling | Insulin |
|---|---|---|
| | factor | Scaling Factor |
| % Time in 4-6.0 mmol/L | 47.98% | 51.34% |
| % Time in 4-7.75 mmol/L | 91.12% | 90.66% |
| Feed rate (mL/hr) | 54.6 | 58.74 |
| Average insulin rate (U/hr) | 3 | 3.82 |

These results confirm that the level of patient care can be increased by incorporating a body size insulin scaling factor from patient age, weight, height, and gender. Time in the 4-6.0 mmol/L band increased from 48% to 51% in trials with and without the insulin scaling factor respectively.

During these studies an improvement in neuromuscular complications and reductions in new infections, ventilator dependency, and ventilator-induced pneumonia were seen compared to the non-controlled population. When using the invention the incidence of ventilator-induced pneumonia in cohorts treated with the invention is reduced when compared to prior cohorts not treated by the invention by 9%.

### Example 5

### Modulation Strategy Incorporating Body Temperature, Renal Function, Urine Output, Blood Pressure, and Catecholamine Dosage

Although models using age, weight, height, and gender provide a better assessment of the overall long-term health of the individual than models not using such parameters (the results being closer to the predicted outcomes), they still did not accurately reflect the current real-time body status. In critical care where patient health can be extremely volatile and change minute to minute, there exists a need for a means to quickly assess the current status of the patient. In order to do so, several key parameters were identified. These key parameters included, but were not limited to, body temperature, renal function, urine output, blood pressure, and catecholamine dosage. These parameters were used to provide a snapshot of the real-time body status of the patient. Each of these variables can be incorporated into the therapy decision process via body status scaling factors. These scaling factors can be incorporated to the algorithm in an identical methodology to the body size insulin scaling factor. The insulin and nutrition dosages are simply multiplied by these additional body status factors.

Body temperature is a continuous signal and verified marker of patient metabolic state. This signal is constantly monitored in the ICU and is used as a primary marker for diagnosis and tracking of disease. A general rule of thumb used in intensive care medicine is metabolic rate decreases by 6% every degree centigrade below normal body temperature and increases by 3% every degree centigrade above normal body temperature. Clinicians must be able to quickly titrate insulin and nutrition to match the metabolic state as described by body temperature. Table 18 shows the results after implementing an insulin and nutrition modulation approach incorporating temperature on patients with a high degree of temperature variability versus the trials without incorporating temperature into the algorithm.

To save using multiple algorithms, temperature was incorporated via two additional body status scaling factors, namely, a temperature-based nutrition scaling factor and a temperature-based insulin scaling factor. Table 7 displays example temperature-based scaling factors.

TABLE 18
Temperature-based scaling factors

| | Temp-based | Temp-based |
|---|---|---|
| | nutrition scaling | insulin scaling |
| Temperature | factor | factor |
| below 36 | 0.88 | 1.14 |
| 36 or above | 1 | 1.00 |
| and below 39 | | |
| 39 or above | 1.07 | 1.14 |

These scaling factors fit the accepted medical doctrine that BMR increases with temperature. As body temperature drops below 36 degrees centigrade, the nutrition scaling factor is reduced and hence the net carbohydrate intake is decreased. The body is unable to utilize as much glucose at these lower temperatures. Therefore, the device would recommend a decreased nutrition rate. Conversely at a body temperature above 39 degrees centigrade the body's metabolic kinetics increase and glucose is utilized at a quicker rate. To compensate for this increase in metabolic demand the device would recommend an increased nutrition rate.

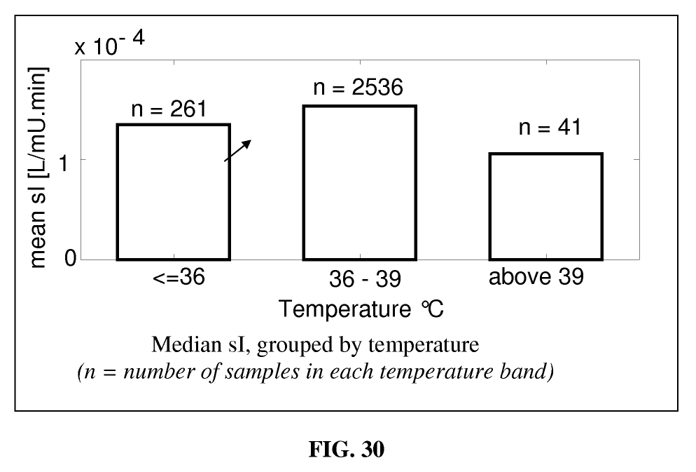

In order to determine the appropriate insulin scaling factor based on body temperature a quantitative analysis of 36 critically ill patients was conducted to provide an extended analysis of the effect of body temperature on insulin sensitivity. The median value of model-fitted insulin sensitivity (herein referred to as S_{I}), grouped by body temperature is shown in FIG. 30. Data was collected for 36 patients from the Christchurch Hospital Intensive Care Unit in Christchurch, New Zealand where there was a possibility the patient had sepsis. Insulin sensitivity data is presented for periods only where the patient was receiving insulin.

It is clear from FIG. 30 that the S_{I} profile at different temperatures approximates a normal or bell curve. S_{I} is highest at normal body temperatures or approximately 36-39 degrees centigrade. However S_{I} decreases significantly after body temperature exceeds 39 degrees centigrade or drops below 36 degrees centigrade. Hence, insulin is used most effectively in the normothermic range, but it is less effective outside this range. Consequently more insulin must be used at these extreme temperatures to combat the decrease in insulin sensitivity or increased insulin resistance. As a result the insulin scaling factor based on body temperature is 1.14 for temperatures greater than 39 degrees centigrade or less than 36 degrees centigrade. The insulin scaling factor is equal to 1.0 at normal body temperatures.

The median value of S_{I} differs between temperature bands (P=0.002, Kruskal-Wallis test adjusted for ties). Post-hoc analysis shows significant differences in S_{I} for hours when temperature was below 36° C. as well as 39° C. or above compared to the S_{I} when temperature is between 36-39° C. (P=0.005 and P=0.03 respectively, Mann-Whitney U test). This analysis shows that there is a statistically significant drop in insulin sensitivity when body temperature is above 39° C. or below 36° C. and provides further evidence that temperature is an accurate predictor of current metabolic state and must be taken into account when titrating insulin and nutrition dosages for critically ill patients.

TABLE 19
Magnitude of S_{I} grouped by temperature

| Temperature | Median S_{I}\| | Inter-quartile range |
|---|---|---|
| range [° C.] | [L/mU · min] | [L/mU · min] |
| <=36 | 13.5 × 10⁻⁵ | 4.33-23.0 × 10⁻⁵ |
| 36-39 | 15.4 × 10⁻⁵ | 8.65-23.8 × 10⁻⁵ |
| 39 or above | 10.6 × 10⁻⁵ | 7.53-16.7 × 10⁻⁵ |

In addition at lower temperatures the insulin effects and kinetics are slowed down, thereby requiring more insulin to get the same effect as observed at normal body temperature. Thus the scaling factor increased with lower body temperature. The outputs of the insulin and nutrition algorithms are simply multiplied by temperature insulin and temperature nutrition factors respectively. From this, it became clear that there existed a need for a quick and easy to use system that accurately incorporates temperature into the clinician's therapy decision. Incorporating temperature brought the results closer to the anticipated results predicted from our models. The study did detect an improvement in on neuromuscular complications, reduction in new infections, ventilator dependency and a reduction in ventilator-induced pneumonia that was unexpected benefit. By taking into account the metabolic condition and varying the insulin and the nutrition the blood glucose of the patient was brought into control and the incidences of ventilator-induced pneumonia was reduced.

TABLE 20
Glycemic control trials incorporating temperature

| | Not Incorporating | Incorporating |
|---|---|---|
| | Temperature | Temperature |
| % Time in 4-6.0 mmol/L | 45.6% | 46.0% |
| % Time in 4-7.75 mmol/L | 86.3% | 86.8% |
| Avg. insulin temperature factor | N/A | 1.01 |
| Avg. nutrition temperature factor | N/A | 0.99 |
| Feed rate (mL/hr) | 53.62 | 52.4 |

Incorporating temperature, using the example scaling factors in Table 18, increased time in the 4-6 mmol/L band from 45.6% to 46.0% and time in the 4-7.75 mmol/L band from 86.3% to 86.6%. The average scaling factors were 1.01 and 0.99. Incorporating temperature has a positive effect on patient control increasing the time patients spent both in the 4-6 mmol/L and 4-7.75 mmol/L band. Alternative temperature scaling factors are possible. The nutrition temperature scaling factor could be any value between 0.2-3. The insulin temperature scaling factor could be any value between 0.2-3.

The study did detect an improvement on neuromuscular complications and reductions in new infections, ventilator dependency, and ventilator-induced pneumonia that was unexpected benefit. By taking into account the metabolic condition and varying the insulin and the nutrition the blood glucose of the patient was brought into control and the incidences of ventilator-induced pneumonia were reduced. When using the invention the incidence of ventilator-induced pneumonia in cohorts treated with the invention is reduced when compared to prior cohorts not treated by the invention by 15%. This is a significant reduction.

The kidney is the major site of insulin clearance from the systemic circulation. Impairment of kidney or renal function is common in the ICU where many patients may have some form of kidney disease or suffer complete renal failure. Clinicians measure renal function of a hospitalized individual at routine and regular intervals via plasma concentrations of creatinine, urea, and electrolytes. Creatinine clearance can be used to calculate the glomerular filtration rate (GFR). Alternatively, an estimate of GFR can be calculated via the concentration of creatinine in the bloodstream and the Modification of Diet in Renal Disease (MDRD) equations. The GFR is the volume of fluid filtered from the renal capillaries per unit time. The MDRD equation is given by:

GFR(mL/min/1.73 m²)=186*[serum creatinine(μmol/L)*0.011312]^{-1.154}*[age]^{-0.203}*[1.212 if black]*[0.742 if female] (6)

Additional metrics related to renal function include aminoglycoside dosage, serum aminoglycoside concentration, and noradrenaline usage. Varying patient GFR results were easily incorporated into the decision support algorithms via additional scaling factors. Table 21 presents the optimal scaling factors verified via extensive experimentation.

TABLE 21
Kidney factors used in decision support algorithm

| | Kidney | Kidney |
|---|---|---|
| GFR | nutrition | insulin |
| (mL/min/1.73 m\hat{ }2) | factor | factor |
| >90 | 1 | 1 |
| 60-89 | 1.05 | 0.95 |
| 30-59 | 1.11 | 0.9 |
| <30 | 1.18 | 0.85 |

As GFR decreases a smaller volume of fluid is filtered by the kidneys per unit time, thereby requiring less insulin to be cleared from the system. Therefore as GFR decreases more insulin remains in the body in circulation, and the insulin administered to the patient should be decreased. The nutrition rate should be slightly increased to account for the blood glucose reducing capability of the extra insulin in the body.

The kidney factors are multiplied by the insulin and nutrition outputs from the algorithm to give patient specific therapy dosages titrated to match patient kidney function. The clinician is provided a quick and easy means to incorporate kidney function into the therapy process. Here, kidney function was assessed via the GFR calculated from creatinine clearance. Alternative measurements could be used to assess renal function including, but not limited to, aminoglycoside dosage, serum aminoglycoside concentration, or urea concentration.

Table 22 shows glucose control results after incorporating patient GFR as calculated via the MDRD equation from creatinine clearance values on patients with high GFR variation.

TABLE 22
Glycemic control trials incorporating GFR

| | GFR Not Used | GFR Used |
|---|---|---|
| % Time in 4-6.0 mmol/L | 45.66% | 47.14% |
| % Time in 4-7.75 mmol/L | 86.34% | 86.42% |
| Avg. kidney insulin factor | N/A | 1.09 |
| Avg. kidney nutrition factor | N/A | 0.92 |

It is clear from these results that there is an increase in time in the 4-6 mmol/L and 4-7.75 mmol/L bands by incorporating GFR.

Hourly urine output was incorporated in a similar process. Insulin causes the retention of sodium, which causes fluid retention, which is manifested in a decrease in hourly urine output. Changes in hourly urine output are also a clinical marker of the hyperdynamic state of sepsis that leads to a decrease in glucose uptake and storage in comparison with healthy individuals. Urine scaling factors can easily be used by the nutrition and insulin algorithm.

The urine factors utilized in conjunction with the baseline algorithms are displayed in Table 23.

TABLE 23
Optimized urine nutrition and insulin factors

| | Urine nutrition | Urine insulin |
|---|---|---|
| Hourly urine output | factor | factor |
| More than 120 mL/hour | 0.83 | 1.2 |
| At least 80 mL/hour | 1.00 | 1 |
| and less than 120 | | |
| Less than 80 mL/hour | 1.05 | 0.95 |

If the urine level deviates significantly from the hourly norm, changes should be made to both the nutrition and insulin dose. As urine increases more insulin is flushed out of the system and hence the urine insulin factor increases. The converse is true as urine decreases. With regard to nutrition, if urine output is high the body will attempt to flush out excess glucose through the kidneys and hence the urine nutrition factor is reduced.

Hourly urine output is regularly recorded and measured every hour in the intensive care unit and this variable was utilized in a set of trials presented in Table 24.

TABLE 24
Glycemic control trials incorporating urine output

| | Urine | Urine |
|---|---|---|
| | output | output |
| | not used | used |
| % Time in 4-6.0 mmol/L | 45.66% | 47.00% |
| % Time in 4-7.75 mmol/L | 86.34% | 85.92% |
| Avg Hourly Urine | 100 ml | 100 ml |
| Avg Urine Nutrition Factor | N/A | 0.996 |
| Avg Urine Insulin Factor | N/A | 1.004 |

These results demonstrate that one can increase the time a patient's blood glucose concentration is in the 4-6.0 mmol/L band by using urine as a precursor indicator of metabolic state.

Blood pressure is also constantly monitored in the ICU and is a measure of the pressure exerted perpendicular to the walls of the blood vessels. Blood pressure is not static and may undergo variation from one heart beat to the next and in response to stress, nutrition factors, drugs, or disease. High levels of insulin in the body can cause several problems, one of them being high blood pressure. One of the roles of insulin is to assist the storing of excess nutrients. Insulin also plays a role in storing magnesium. If the cells of a patient's body become resistant to insulin (insulin resistance increases), the body is unable to store magnesium and any magnesium present is lost through urination. Intra-cellular magnesium relaxes muscles. When a patient cannot store magnesium because the cell is resistant, they lose magnesium and their blood vessels constrict. This causes an increase in blood pressure. Insulin sensitivity has been correlated to arterial hypertension or high blood pressure in the arteries. Incorporating blood pressure into the metabolic control algorithm provides information on the relative levels of insulin currently in the patient's blood.

Blood pressure was incorporated via a blood pressure factor calculated from hourly blood pressure values. Table 25 displays optimal values of the blood pressure factor.

TABLE 25
Blood pressure factor values used in glycemic control trials
Blood pressure factor calculation

| | |
|---|---|
| 130 or greater (mmHg) | 1.25 |
| 110 to 129 (mmHg) | 1.1 |
| 70-109 (mmHg) | 1 |
| 50-69 (mmHg) | 1.1 |
| 49 or lower (mmHg) | 1.25 |

As blood pressure deviates from the norm (70-109 mmHg), insulin and nutrition rates are scaled via the blood pressure factor. Blood pressure was proven to be an effective input in metabolic control in a series of trials presented in Table 26.

TABLE 26
Glycemic control trials incorporating blood pressure data

| | BP not used | BP used |
|---|---|---|
| % Time in 4-6.0 mmol/L | 45.66% | 47.56% |
| % Time in 4-7.75 mmol/L | 86.34% | 86.72% |
| Avg blood pressure factor | N/A | 0.97 |

Time in the 4-6 mmol/L band and the 4-7.75 mmol/L band both increased. Hence blood pressure is an effective input to the metabolic control device.

Medication regimes also have a significant affect on human metabolism. Catecholamines are hormones released by the adrenal glands in situations of stress such as psychological stress or low blood glucose levels. Catecholamines cause general physiological changes that prepare the body for physical activity (fight-or-flight response). Some typical effects are increases in heart rate, blood pressure, blood glucose levels, and a general reaction of the sympathetic nervous system. Synthetic catecholamines are commonly used in intensive care as drugs, such as epinephrine, to increase peripheral resistance via alpha-stimulated vasoconstriction in cardiac arrest and other cardiac dysrhythmias.

Because of its suppressive effect on the immune system, epinephrine is used to treat anaphylaxis and sepsis. Allergy patients undergoing immunotherapy may receive an epinephrine rinse before the allergen extract is administered, thus reducing the immune response to the administered allergen. It is also used as a bronchodilator for asthma. Many catecholamines are used clinically. These catecholamines include, but are not limited to, dopamine, epinephrine (adrenaline), norepinephrine (noradrenaline), orciprenaline, dobutamine, and isoproterenol. Given the affect of these drugs on metabolic status any or all of these drugs could be taken into account when titrating therapy to match metabolic demand.

Adrenaline was incorporated into the algorithm as a catecholamine insulin factor and a catecholamine nutrition factor, the values of which are displayed in Table 27.

TABLE 27
Glycemic control trials incorporating adrenaline usage

| | Catecholamine | Catecholamine |
|---|---|---|
| | nutrition | Insulin |
| Category | factor | Factor |
| Adrenaline not being administered | 1 | 1 |
| Adrenaline administered | 0.83 | 1.2 |

When adrenaline or noradrenaline is being administered, the concentration of catecholamine hormones in the blood is increased and the liver releases additional glucose into the blood. Hence blood glucose levels are driven upwards and the nutrition regime should be decreased. Insulin dosages are increased to counteract these high blood glucose levels. Trials were conducted incorporating adrenaline usage into the therapy decisions. Table 16 displays the results incorporating catecholamine dosage (in this example adrenaline) into the decision support algorithm.

TABLE 28
Glycemic control trials incorporating catecholamine (adrenaline) usage

| | Adrenaline or | Adrenaline or |
|---|---|---|
| | Noradrenaline | Noradrenaline |
| | not used | Used |
| % Time in 4-6.0 mmol/L | 45.66% | 47.80% |
| % Time in 4-7.75 mmol/L | 86.34% | 86.66% |
| Avg. Cateholamine Nutrition Factor | N/A | 0.94 |

By adjusting the nutrition and insulin dosages to match catecholamine dosage, the clinician can increase blood glucose control. Time in the 4-6 mmol/L band and time in the 4-7.75 mmol/L band both increased respectively.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A method of providing a blood glucose therapy for a critically ill patient, said method comprising the steps of:
calculating a baseline nutritional feed requirement for said patient based on an algorithm that incorporates at least a weight of said patient;
determining a first body temperature of said patient;
determining a first blood glucose level of said patient;
determining at least a second body temperature of said patient after a preselected time period;
determining at least a second blood glucose level of said patient after a preselected time period; and
comparing said at least second blood glucose level to said first blood glucose level;
administering one of an amount of nutritional feed to said patient and an amount of insulin to said patient;
wherein said amount of nutritional feed administered to said patient is based on a first decrease in blood glucose level and a predetermined feed algorithm that incorporates said second blood glucose level and said patient's baseline nutritional feed requirement; and
wherein said amount of insulin administered to said patient is based on a second decrease in blood glucose level that is less than said first decrease in blood glucose level and a predetermined insulin algorithm that incorporates said at least second blood glucose level and said patient's weight.

2. The method of claim 1, wherein said step of administering one of an amount of nutritional feed to said patient and an amount of insulin to said patient comprises maintaining said blood glucose level of said patient in the range of 4.0 mmol/L to 7.75 mmol/L.

3. The method of claim 1, wherein said preselected time period is 2 hours or less.

4. The method of claim 1, wherein said method is used to reduce the occurrence of ventilator-induced pneumonia by controlling a patient's blood glucose level.

5. The method of claim 1, wherein said patient was not diagnosed with diabetes prior to becoming critically ill and in which said patient is not otherwise considered to be either a type I or type II diabetic.

6. The method of claim 1, wherein said nutritional feed and said insulin administered to said patient is determined using a linear slide rule calculator or a circular slide rule calculator.

7. The method of claim 1, wherein said nutritional feed and said insulin administered to said patient is determined using an electronic computing device.

8. The method of claim 1, wherein blood glucose levels of said patient are determined repetitively.

9. The method of claim 1, wherein said predetermined feed algorithm incorporates said patient's one or more metabolic markers selected from the group of body temperature, renal function, urine output, catecholamine dosage, blood pressure and one or more patient status variables selected from the group consisting of age, weight and height, gender, and body frame size.

10. The method of claim 1, wherein said step of administering one of an amount of nutritional feed to said patient and an amount of insulin to said patient is effected using a pump.

11. A method of determining a nutritional input and an insulin input for a discrete time period for a patient that is critically ill, said method comprising the steps of:
determining an insulin scaling factor;
determining a nutrition scaling factor;
determining a precursor of a metabolic state of said patient based on a selected metabolic marker;
determining a blood glucose level of said patient;
entering data indicative of said insulin scaling factor, said nutrition scaling factor, said precursor of a metabolic state of said patient, and said blood glucose level of said patient into an electronic calculation means;
calculating one of an insulin amount to be administered to said patient and a nutrition amount to be administered to said patient;
wherein said insulin amount is based on said entered information and wherein said nutrition amount is based on said entered information.

12. The method of claim 11, wherein said selected metabolic marker of said patient is selected from the group consisting of body temperature, renal function, urine output, blood pressure, catecholamine dosage, age, weight, height, gender, and combinations of the foregoing.

13. The method of claim 11, wherein said patient is at least one of diabetic or stress-induced hyperglycemic.

14. A digital computational device for assisting a clinician in determining a therapy for a patient, said device comprising:
means for calculating a recommended nutrition rate from a first corresponding algorithm, said first corresponding algorithm comprising calculating a first value from one or more physiological parameters specific to said patient and one or more real-time precursor status indicators; and
means for calculating a recommended insulin dosage from a second corresponding algorithm, said second corresponding algorithm comprising calculating a second value from one or more physiological parameters specific to said patient and one or more real-time precursor status indicators;
wherein said calculated recommended nutrition rate and said calculated recommended insulin dosage are incorporated into said therapy for obtaining and maintaining metabolic homeostasis in said patient.

15. The device of claim 14, wherein said one or more physiological parameters and said one or more real-time precursor status indicators are input as digitized data.

16. The device of claim 14, wherein said one or more physiological parameters specific to said patient corresponds to at least one of said patient's age, gender, height and weight, body temperature, previous nutrition dosage, previous insulin dosage, and current blood glucose level.

17. The device of claim 14, wherein said one or more real-time precursor status indicators includes scaling factors indicative of values selected from the group consisting of body temperature, renal function, urine output, blood pressure, medication history, previous nutrition dosage, previous insulin dosage, current blood glucose level, and combinations of the foregoing.

18. A method of establishing metabolic homeostasis in a patient having hyperglycemic blood glucose levels, said method comprising the steps of:
inputting a first physiological parameter into an electronic calculation device, said first physiological parameter comprising a factor specific to said patient;
inputting a second physiological parameter into said electronic calculation device, said second physiological parameter comprising a real-time parameter comprising a factor indicative of said patient's metabolism;
inputting a third physiological parameter into said electronic calculation device, said third physiological parameter comprising at least one factor derived from current and past data relating to said patient; and
calculating a recommended dosing rate to be administered to said patient from said input parameters.

19. The method of claim 18, further comprising controlling said patient's blood glucose level to reduce the occurrence of ventilator-induced pneumonia in said patient.

20. The method of claim 18, further comprising providing a confirmation signal of said calculated recommended dosing rate to said electronic calculation device and communicating said calculated recommended dosing rate to a pump for infusion into a patient.

21. A method of using a digital computational device to establish metabolic homeostasis in a patient having a hyperglycemic blood glucose level, said method comprising:
inputting information specific to said patient into said digital computational device, said information being indicative of at least one of the age, gender, and body size of said patient;
inputting a physiological parameter into said digital computational device, said physiological parameter comprising a real-time parameter comprising a factor indicative of said patient's metabolism;
inputting a blood glucose value of said patient and prior nutrition and insulin dosages delivered to said patient relating to a discrete time period into said digital computational device; and
calculating a recommended dosing rate to be administered to said patient, said recommended dosing rate being calculated from said input information specific to said patient, said input physiological parameter, and said input blood glucose value and said prior nutrition and insulin dosages.

22. The method of claim 21, further comprising receiving a confirmatory signal from said electronic device, said confirmatory signal confirming said recommended dosing rate.

23. The method of claim 21, further comprising communicating said recommended dosing rate to a pump configured to infuse said patient with one of insulin and nutrition.

24. The method of claim 21, wherein said step of inputting a physiological parameter into said digital computational device comprises reading a body temperature of said patient via a device connected to said digital computational device.

25. The method of claim 21, wherein said step of inputting a blood glucose value into said digital computational device comprises reading said blood glucose value of said patient via a device connected to said digital computational device.
